(19) United States
(12) Patent Application Publication
(10) Pub. No.: US 2024/0411226 A1
(43) Pub. Date: Dec. 12, 2024

(54) RESIST COMPOSITION, METHOD FOR FORMING RESIST PATTERN, POLYMER COMPOUND, AND COMPOUND

(71) Applicant: TOKYO OHKA KOGYO CO., LTD., Kawasaki-shi (JP)

(72) Inventors: Hiroki Kato, Kawasaki-shi (JP); Shuichi Ishii, Kawasaki-shi (JP); Tetsuo Fujinami, Kawasaki-shi (JP)

(21) Appl. No.: 18/694,868

(22) PCT Filed: Oct. 6, 2022
(86) PCT No.: PCT/JP2022/037395

§ 371 Date: Mar. 22, 2024

(30) Foreign Application Priority Data
Oct. 11, 2021 (JP) 2021-166784

(51) Int. Cl. G03F 7/039; G03F 7/038; G03F 7/20

(52) U.S. Cl. CPC G03F 7/0397; G03F 7/0382; G03F 7/2004

(57) ABSTRACT

A resist composition which generates an acid upon exposure to light and whose solubility in a developing solution is changed under action of an acid, the resist composition containing a resin component whose solubility in a developing solution is changed under action of an acid, in which the resin component has a constitutional unit derived from a compound represented by General Formula (a0) in which W represents a polymerizable group, Ar represents an aromatic hydrocarbon group, —OH represents a hydroxy group, La⁰ represents a divalent linking group, Ya⁰ represents a single bond or a divalent linking group, Ra⁰¹ and Ra⁰² each independently represents a hydrogen atom, a fluorine atom, or a fluorinated alkyl group, n0 represents an integer in a range of 1 to 4, m represents an integer of 1 or more, and M^{m+} represents an m-valent organic cation.

### TECHNICAL FIELD

The present invention relates to a resist composition, a method of forming a resist pattern, a polymer compound, and a compound.

Priority is claimed on Japanese Patent Application No. 2021-166784, filed Oct. 11, 2021, the content of which is incorporated herein by reference.

### BACKGROUND ART

In recent years, in the production of semiconductor elements and liquid crystal display elements, advances in lithography technologies have led to rapid progress in the field of pattern miniaturization. Typically, these pattern miniaturization techniques involve shortening the wavelength (increasing the energy) of the light source for exposure.

Resist materials have been required to have lithography characteristics such as sensitivity to these light sources for exposure and resolution capable of reproducing a fine-sized pattern.

As a resist material that satisfies these requirements, a chemically amplified resist composition containing a base material component whose solubility in a developing solution is changed under action of an acid and an acid generator component that generates an acid upon exposure to light is used in the related art.

In the chemically amplified resist composition, a resin having a plurality of constitutional units is typically used in order to improve lithography characteristics and the like.

In addition, in the formation of the resist pattern, the behavior of an acid generated from an acid generator component upon exposure to light serves as an element that greatly affects the lithographic characteristics.

Various kinds of acid generators have been suggested as the acid generator used in a chemically amplified resist composition. For example, onium salt-based acid generators such as an iodonium salt and a sulfonium salt, oxime sulfonate-based acid generators, diazomethane-based acid generators, nitrobenzyl sulfonate-based acid generators, iminosulfonate-based acid generators, and disulfone-based acid generators are known.

In recent years, a chemically amplified resist composition that contains, in the structure, a resin component having an acid generating group which generates an acid upon exposure to light and an acid decomposable group whose polarity is increased under action of an acid has been suggested as the chemically amplified resist composition. For example, in Patent Document 1, a resist composition containing a resin component having a constitutional unit containing an anion group at a side chain terminal has been suggested.

### CITATION LIST

### Patent Document

[Patent Document 1]
Japanese Unexamined Patent Application. First Publication No. 2014-152122

### SUMMARY OF INVENTION

### Technical Problem

With the further progress of the lithography technology and resist pattern fining, for example, it is aimed to form a fine pattern with a size of several tens of nanometers in lithography by EUV and EB. As described above, in a case where the resist pattern dimensions are decreased, reduction in roughness and high sensitivity to a light source for exposure are required.

A resist composition of the related art as described in Patent Document 1 has room for improvement from the viewpoint of achieving both the sensitivity and lithography characteristics such as LWR.

The present invention has been made in consideration of the above-described circumstances, and an object thereof is to provide a resist composition capable of increasing the sensitivity and having satisfactory lithography characteristics such as LWR and a method for forming a resist pattern using the resist composition.

### Solution to Problem

In order to achieve the above-described object, the present invention adopts the following configurations.

That is, the first aspect according to the present invention is a resist composition which generates an acid upon exposure to light and whose solubility in a developing solution is changed under action of an acid, the resist composition containing: a resin component (A1) whose solubility in a developing solution is changed under action of an acid, in which the resin component (A1) has a constitutional unit (a0) derived from a compound represented by General Formula (a0).

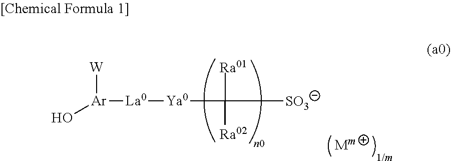

[In the formula, W represents a polymerizable group. Ar represents an aromatic hydrocarbon group which may have a substituent. —OH represents a hydroxy group. La⁰ represents a divalent linking group. Ya⁰ represents a single bond or a divalent linking group which may have a hetero atom. Ra⁰¹ and Ra⁰² each independently represent a hydrogen atom, a fluorine atom, or a fluorinated alkyl group. n0 represents an integer in a range of 1 to 4. m represents an integer of 1 or more, and M^{m+} represents an m-valent organic cation.]

The second aspect according to the present invention is a method for forming a resist pattern including: a step of forming a resist film on a support using the resist composition according to the first aspect; a step of exposing the resist film to light; and a step of developing the resist film exposed to light to form a resist pattern.

The third aspect according to the present invention is a polymer compound having a constitutional unit (a0) derived from the compound represented by General Formula (a0).

The fourth aspect according to the present invention is the compound represented by General Formula (a0).

### Advantageous Effects of Invention

According to the present invention, it is possible to provide a resist composition capable of increasing the sensitivity and having satisfactory lithography characteristics such as LWR and a method for forming a resist pattern using the resist composition.

### DESCRIPTION OF EMBODIMENTS

In the present specification and the scope of the present claims, the term “aliphatic” is a relative concept used with respect to “aromatic” and defines a group, a compound, or the like that has no aromaticity.

The term “alkyl group” includes a monovalent saturated hydrocarbon group that is linear, branched, or cyclic unless otherwise specified. The same applies to the alkyl group of an alkoxy group.

The term “alkylene group” includes a divalent saturated hydrocarbon group that is linear, branched, or cyclic unless otherwise specified.

Examples of the “halogen atom” include a fluorine atom, a chlorine atom, a bromine atom, and an iodine atom.

The term “constitutional unit” means a monomer unit (a monomeric unit) constituting a polymer compound (a resin, a polymer, or a copolymer).

The expression “may have a substituent” includes both a case where a hydrogen atom (—H) is substituted with a monovalent group and a case where a methylene group (—CH₂—) is substituted with a divalent group.

The term “light exposure” is used as a general concept that includes irradiation with any form of radiation.

The term “acid decomposable group” is a group having acid decomposability, in which at least a part of bonds in the structure of the acid decomposable group can be cleaved under action of an acid.

Examples of the acid decomposable group whose polarity is increased under action of an acid include groups which are decomposed under action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (for example, a group in which a hydrogen atom of the OH-containing polar group has been protected by an acid dissociable group).

The term “acid dissociable group” refers to both of (i) a group having acid dissociability, in which a bond between the acid dissociable group and an atom adjacent to the acid dissociable group can be cleaved under action of an acid; and (ii) a group in which after a part of bonds is cleaved under action of an acid, the bond between the acid dissociable group and the atom adjacent to the acid dissociable group can be cleaved by further occurring a decarboxylation reaction.

It is necessary that the acid dissociable group constituting the acid decomposable group is a group which exhibits a lower polarity than the polar group generated by the dissociation of the acid dissociable group. Thus, in a case where the acid dissociable group is dissociated under action of an acid, a polar group that exhibits a higher polarity than the acid dissociable group is generated, thereby increasing the polarity. As a result, the polarity of an entire component (A1) is increased. With the increase in the polarity, the solubility in a developing solution relatively changes. The solubility in a developing solution is increased in a case where the developing solution is an alkali developing solution, whereas the solubility in a developing solution is decreased in a case where the developing solution is an organic developing solution.

The term “base material component” is an organic compound having a film-forming ability. The organic compounds used as the base material component are roughly classified into a non-polymer and a polymer. As the non-polymer, those having a molecular weight of 500 or more and less than 4,000 are typically used. Hereinafter, the term “low-molecular-weight compound” indicates a non-polymer having a molecular weight of 500 or more and less than 4,000. As the polymer, those having a molecular weight of 1,000 or more are typically used. Hereinafter, the term “resin”, “polymer compound”, or “polymer” indicates a polymer having a molecular weight of 1,000 or more. As the molecular weight of the polymer, the weight-average molecular weight in terms of polystyrene according to gel permeation chromatography (GPC) is used.

The expression “constitutional unit to be derived” means a constitutional unit formed by cleavage of a multiple bond between carbon atoms, for example, an ethylenic double bond.

In the “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. In addition, the acrylic acid ester includes itaconic acid diester in which the substituent (R^{αx}) is a substituent containing an ester bond and α-hydroxyacryl ester in which the substituent (R^{αx}) is a hydroxyalkyl group or a group obtained by modifying a hydroxyl group thereof. A carbon atom at the α-position of acrylic acid ester indicates the carbon atom bonded to the carbonyl group of acrylic acid unless otherwise specified.

Hereinafter, acrylic acid ester in which the hydrogen atom bonded to the carbon atom at the α-position has been substituted with a substituent is also referred to as α-substituted acrylic acid ester.

The term “derivative” is used as a concept that includes a compound obtained by substituting a hydrogen atom at the α-position of an target compound with another substituent such as an alkyl group or a halogenated alkyl group, and a derivative thereof. Examples of the derivatives thereof include a derivative in which the hydrogen atom of the hydroxyl group of the target compound in which the hydrogen atom at the α-position may be substituted with a substituent is substituted with an organic group; and a derivative in which a substituent other than a hydroxyl group is bonded to the target compound in which the hydrogen atom at the α-position may be substituted with a substituent. The α-position refers to the first carbon atom adjacent to the functional group unless otherwise specified.

Examples of the substituent that substitutes the hydrogen atom at the α-position of hydroxystyrene include the same as those for R^{αx}.

In the present specification and the scope of the present claims, asymmetric carbons may be present and enantiomers or diastereomers may be present depending on the structures represented by the chemical formula. In that case, these isomers are representatively represented by one chemical formula. These isomers may be used alone or in the form of a mixture.

### (Resist Composition)

The resist composition according to the present embodiment is a resist composition which generates an acid upon exposure to light and whose solubility in a developing solution is changed under action of an acid.

Such resist composition contains a base material component (A) (hereinafter, also referred to as a “component (A)”) whose solubility in a developing solution is changed under action of an acid.

In the present embodiment, the component (A) contains a resin component (A1) (hereinafter, also referred to as a “component (A1)”) whose solubility in a developing solution is changed under action of an acid. The component (A1) has a constitutional unit (a0) derived from a compound represented by General Formula (a0) described later.

In a case where a resist film is formed using the resist composition according to the present embodiment and the formed resist film is subjected to selective light exposure, an acid is generated from the constitutional unit (a0) at an exposed portion of the resist film, and the solubility of the component (A) in a developing solution is not changed at an unexposed portion of the resist film while the solubility of the component (A) in a developing solution is changed by the action of the acid, and thus a difference in solubility in a developing solution occurs between the exposed portion and the unexposed portion. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

In the present specification, a resist composition which forms a positive-tone resist pattern by dissolving and removing the exposed portion of the resist film is referred to as a positive-tone resist composition, and a resist composition which forms a negative-tone resist pattern by dissolving and removing the unexposed portion of the resist film is referred to as a negative-tone resist composition. The resist composition according to the present embodiment may be a positive-tone resist composition or a negative-tone resist composition. In addition, the resist composition according to the present embodiment may be used in an alkali developing process using an alkali developing solution in the developing treatment in a case of forming a resist pattern or may be used in a solvent developing process using a developing solution containing an organic solvent (organic developing solution) in the developing treatment.

### <Component (A)>

In the resist composition according to the present embodiment, it is preferable that the component (A) has a resin component (A1) whose solubility in a developing solution is changed due to the action of an acid (hereinafter, also referred to as a “component (A1)”). In the alkali developing process and the solvent developing process, since the polarity of the base material component before and after the light exposure is changed by using the component (A1), an excellent development contrast can be obtained.

As the component (A), another polymer compound and/or a low-molecular-weight compound may be used in combination with the component (A1).

In the resist composition according to the present embodiment, the component (A) may be used alone or in combination of two or more kinds thereof.

In regard to component (A1)

The component (A1) is a resin component whose solubility in a developing solution is changed due to the action of an acid.

The component (A1) preferably has a constitutional unit (a0) and a constitutional unit (a1) containing an acid decomposable group whose polarity is increased under action of an acid, which will be described later.

The component (A1) may have other constitutional units as necessary in addition to the constitutional unit (a0) and the constitutional unit (a1).

<<Constitutional unit (a0)>>

The constitutional unit (a0) is a constitutional unit derived from a compound represented by General Formula (a0).

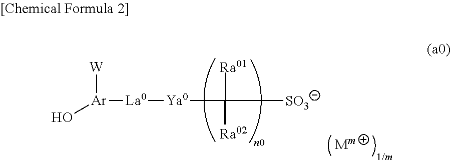

[In the formula, W is a polymerizable group. Ar is an aromatic hydrocarbon group which may have a substituent. —OH is a hydroxy group. La⁰ represents a divalent linking group. Ya⁰ represents a single bond or a divalent linking group which may have a hetero atom. Ra⁰¹ and Ra⁰² each independently represent a hydrogen atom, a fluorine atom, or a fluorinated alkyl group. n0 represents an integer in a range of 1 to 4. m represents an integer of 1 or more, and M^{m+} represents an m-valent organic cation.]

In General Formula (a0), the “polymerizable group” as W is a group that enables a compound having the polymerizable group to be polymerized by radical polymerization or the like, and refers to a group containing a multiple bond between carbon atoms, such as an ethylenic double bond.

The expression “derived from a compound represented by General Formula (a0)” means that a multiple bond in a polymerizable group is cleaved to form a main chain. For example, in a case of a monomer having an ethylenic double bond, the expression means that an ethylenic double bond is cleaved and a single bond between carbon atoms forms the main chain.

Examples of the polymerizable group as W include a vinyl group, an allyl group, acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonylfluorobutylacryloyl group, a vinyl ether group, a fluorine-containing vinyl ether group, an allyl ether group, a fluorine-containing allyl ether group, a styryl group, and a vinylnaphthyl group, a fluorine-containing styryl group, a fluorine-containing vinylnaphthyl group, a norbornenyl group, a fluorine-containing norbornenyl group, a silyl group, and the like. The polymerizable group may be a group formed of only a polymerizable group or a group formed of a polymerizable group and a group other than the polymerizable group. Examples of the group other than the polymerizable group include a divalent hydrocarbon group which may have a substituent and a divalent linking group containing a hetero atom.

Suitable examples of W include a group represented by Chemical Formula: C(R^{X11})(R^{X12})═C(R^{X13})—Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

In the chemical formula described above, the alkyl group having 1 to 5 carbon atoms as R^{X11}, R^{X12}, and R^{X13} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, a neopentyl group, and the like. The halogenated alkyl group having 1 to 5 carbon atoms is a group in which a part of or all hydrogen atoms in the alkyl group having 1 to 5 carbon atoms are substituted with halogen atoms. Examples of the halogen atom include a fluorine atom, a chlorine atom, a bromine atom, and an iodine atom. Among these, a fluorine atom is particularly preferable.

Among these, R^{X11} and R^{X12} are each preferably a hydrogen atom, an alkyl group having 1 to 5 carbon atoms, or a fluorinated alkyl group having 1 to 5 carbon atoms, and in terms of industrial availability, a hydrogen atom or a methyl group is more preferable, and a hydrogen atom is particularly preferable.

In addition, R^{X11} is preferably a hydrogen atom, an alkyl group having 1 to 5 carbon atoms, or a fluorinated alkyl group having 1 to 5 carbon atoms, and in terms of industrial availability, a hydrogen atom or a methyl group is more preferable.

In the chemical formula, the divalent linking group as Ya^{x0} is not particularly limited, and suitable examples thereof include a divalent hydrocarbon group which may have a substituent and a divalent linking group having a hetero atom.

Divalent Hydrocarbon Group which May have Substituent:

In a case where Ya^{x0} represents a divalent hydrocarbon group which may have a substituent, the hydrocarbon group may be an aliphatic hydrocarbon group or an aromatic hydrocarbon group.

### Aliphatic Hydrocarbon Group as Ya^{x0}

The aliphatic hydrocarbon group indicates a hydrocarbon group that has no aromaticity. The aliphatic hydrocarbon group may be saturated or unsaturated. Typically, the aliphatic hydrocarbon group is preferably saturated.

Examples of the aliphatic hydrocarbon group include a linear or branched aliphatic hydrocarbon group or an aliphatic hydrocarbon group having a ring in the structure thereof, and the like.

### Linear or Branched Aliphatic Hydrocarbon Group

The linear aliphatic hydrocarbon group preferably has 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As an alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, a carbonyl group, and the like.

### Aliphatic Hydrocarbon Group Containing Ring in Structure Thereof

Examples of the aliphatic hydrocarbon group having a ring in the structure thereof include a cyclic aliphatic hydrocarbon group which may have a substituent having a hetero atom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. The monocyclic alicyclic hydrocarbon group is preferably a group in which two hydrogen atoms have been removed from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable, and specific examples of the polycycloalkane include a polycycloalkane having a condensed ring-based polycyclic skeleton, such as a ring structure having decalin, perhydroazulene, perhydroanthracene, or a steroid skeleton.

In addition, as the aliphatic hydrocarbon group having a ring in the structure described above, Ya⁰ may share a carbon atom in the polymerizable group as W to form an aliphatic hydrocarbon group having a ring in the above-described structure.

A cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the above-described substituent, an alkyl group having 1 to 5 carbon atoms is preferable, and a methyl group, an ethyl group, a propyl group, an n-butyl group, or a tert-butyl group is most preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom as the substituent include a fluorine atom, a chlorine atom, a bromine atom, and an iodine atom. Among these, a fluorine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a hetero atom. As the substituent having a hetero atom, —O—, —C(═O)—O—, —S—, —S(═O)₂—, or —S(═O)₂—O— is preferable.

### Aromatic Hydrocarbon Group as Ya^{x0}

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Provided that the number of carbon atoms in a substituent is not included in the number of carbon atoms. Specific examples of the aromatic ring include aromatic hydrocarbon rings such as benzene, naphthalene, anthracene, and phenanthrene; and aromatic heterocyclic rings in which some of the carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group (an arylene group or a heteroarylene group) obtained by removing two hydrogen atoms from the above-described aromatic hydrocarbon ring or the above-described aromatic heterocyclic ring; a group obtained by removing two hydrogen atoms from an aromatic compound (for example, biphenyl or fluorene) having two or more aromatic rings; and a group (for example, a group obtained by further removing one hydrogen atom from an aryl group in the arylalkyl group such as a benzyl group, a phenethyl group, a 1-naphthylmethyl group, a 2-naphthylmethyl group, a 1-naphthylethyl group, or a 2-naphthylethyl group) obtained by substituting one hydrogen atom of a group (an aryl group or a heteroaryl group) obtained by removing one hydrogen atom from the above aromatic hydrocarbon ring or the above aromatic heterocyclic ring, with an alkylene group. The alkylene group bonded to the aryl group or the heteroaryl group, which are described above, has preferably 1 to 4 carbon atoms, more preferably 1 or 2 carbon atoms, and particularly preferably 1 carbon atom.

In the above-described aromatic hydrocarbon group, a hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxyl group.

As the alkyl group as the above-described substituent, an alkyl group having 1 to 5 carbon atoms is preferable, and a methyl group, an ethyl group, a propyl group, an n-butyl group, or a tert-butyl group is most preferable.

Examples of the alkoxy group, the halogen atom, and the halogenated alkyl group as the substituents include those exemplified as the substituent that substitutes a hydrogen atom in the cyclic aliphatic hydrocarbon group.

### Divalent Linking Group Containing Hetero Atom:

In a case where Ya⁰ represents a divalent linking group having a hetero atom, preferred examples of the linking group include —O—, —C(═O)—O—, —C(═O)—, —O—C(═O)—O—, —C(═O)—NH—, —NH—, —NH—C(═NH)— (H may be substituted with a substituent such as an alkyl group and an acyl group), —S—, —S(═O)₂—, —S(═O)₂—O—, a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²¹— [In the formulae, Y²¹ and Y²² each independently represent a divalent hydrocarbon group which may have a substituent, O represents an oxygen atom, and m″ represents an integer in a range of 0 to 3], and the like.

In a case where the divalent linking group having a hetero atom is —C(═O)—NH—, —C(═O)—NH—C(═O)—. —NH—, or —NH—C(═NH)—, H may be substituted with a substituent such as an alkyl group or an acyl group. The substituent (an alkyl group, an acyl group, or the like) has preferably 1 to 10 carbon atoms, more preferably 1 to 8 carbon atoms, and particularly preferably 1 to 5 carbon atoms.

In General Formulae —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²—, Y²¹ and Y²² each independently represent a divalent hydrocarbon group which may have a substituent. Examples of the divalent hydrocarbon group include the same groups as those for the divalent hydrocarbon group which may have a substituent, described in the section of the divalent linking group above.

As Y²¹, a linear aliphatic hydrocarbon group is preferable, a linear alkylene group is more preferable, a linear alkylene group having 1 to 5 carbon atoms is still more preferable, and a methylene group or an ethylene group is particularly preferable.

Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer in range of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Among the above, Ya^{x0} is preferably an ester bond [—C(═O)—O—, —O—C(═O)—], an ether bond (—O—), a linear or branched alkylene group, or a combination thereof, or a single bond, and among these, an ester bond [—C(═O)—O—, —O—C(═O)—], a linear or branched alkylene group, or a combination thereof, or a single bond is more preferable, and a single bond is particularly preferable.

In General Formula (a0), the aromatic carbonized group as Ar is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include an aromatic hydrocarbon ring such as benzene, naphthalene, anthracene, phenanthrene, azulene, acenaphthylene, or the like; an aromatic heterocyclic ring in which a part of carbon atoms constituting the aromatic hydrocarbon ring is substituted with hetero atoms; and the like. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ar include a group obtained by removing three hydrogen atoms from the aromatic hydrocarbon ring or the aromatic heterocyclic ring; a group obtained by removing three hydrogen atoms from an aromatic compound having two or more aromatic rings (biphenyl, fluorene, or the like); and a group obtained by further removing one hydrogen atom from a group in which one hydrogen atom of the aromatic hydrocarbon ring or the aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aromatic hydrocarbon ring or aromatic heterocyclic ring preferably has 1 to 4 carbon atoms, more preferably has 1 or 2 carbon atoms, and particularly preferably has 1 carbon atom.

Among the above, Ar is preferably a group obtained by removing three hydrogen atoms from an aromatic hydrocarbon ring such as benzene, naphthalene, anthracene, or phenanthrene, more preferably a group obtained by removing three hydrogen atoms from benzene or naphthalene, and still more preferably a group obtained by removing three hydrogen atoms from benzene.

In General Formula (a0), the aromatic hydrocarbon group as Ar has at least one hydroxy group (—OH) as a substituent. That is, Ar has at least one phenolic hydroxyl group. The bonding position of the hydroxy group in Ar is not particularly limited, but the para-position of W is preferable.

Examples of the substituent which the aromatic hydrocarbon group as Ar may have include a hydroxyl group, an alkyl group, an alkoxy group, a carboxy group, an alkoxycarbonyl group, an oxo group, an amino group, an alkylamino group, a cyano group, a mercapto group, an alkylthio group, a sulfo group, and the like.

In General Formula (a0), examples of the divalent linking group as La⁰ include a non-hydrocarbon-based oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and a combination of the non-hydrocarbon-based oxygen atom-containing linking group and an alkylene group. A sulfonyl group (—SO₂—) may be further linked to the combination. Examples of such divalent linking group include linking groups each represented by General Formulae (L-al-1) to (L-al-8) shown below. In General Formulae (L-al-1) to (L-al-8), V′¹⁰¹ in General Formulae (L-al-1) to (L-al-8) is bonded to Ar in General Formula (a0).

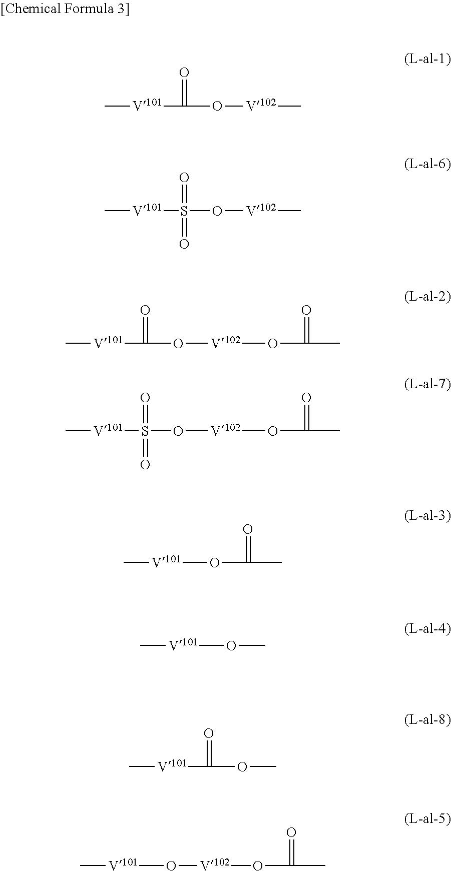

[In the formulae, V′¹⁰¹ represents a single bond or an alkylene group having 1 to 5 carbon atoms, and V′¹⁰² represents a divalent saturated hydrocarbon group having 1 to 30 carbon atoms.]

The divalent saturated hydrocarbon group as V′¹⁰² is preferably an alkylene group having 1 to 30 carbon atoms, more preferably an alkylene group having 1 to 10 carbon atoms, and still more preferably an alkylene group having 1 to 5 carbon atoms.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

In addition, a part of methylene group in the alkylene group as V′¹⁰¹ and V′¹⁰² may be substituted with a divalent aliphatic cyclic group having 5 to 10 carbon atoms. The aliphatic cyclic group is preferably a divalent group obtained by removing one hydrogen atom from the cyclic aliphatic hydrocarbon group (a monocyclic aliphatic hydrocarbon group or a polycyclic aliphatic hydrocarbon group) as Ra′³ in General Formula (a1-r-1), and more preferably a cyclohexylene group, a 1,5-adamantylene group, or a 2,6-adamantylene group.

La⁰ is preferably a divalent linking group containing an ester bond or a divalent linking group containing an ether bond, more preferably linking groups each represented by General Formulae (L-al-1) to (L-al-5) or (L-al-8), and still more preferably a linking group represented by (L-al-3) or (L-al-8).

In General Formula (a0), the bonding position of La⁰ in Ar is not particularly limited, but is preferably the meta position of W.

In General Formula (a), the divalent linking group as Ya⁰ is not particularly limited, and suitable examples thereof include a divalent hydrocarbon group which may have a substituent and a divalent linking group having hetero atoms.

Divalent Hydrocarbon Group which May have Substituent:

In a case where Ya⁰ represents a divalent hydrocarbon group which may have a substituent, the hydrocarbon group may be an aliphatic hydrocarbon group or an aromatic hydrocarbon group.

### Aliphatic Hydrocarbon Group as Ya⁰

The aliphatic hydrocarbon group indicates a hydrocarbon group with no aromaticity. The aliphatic hydrocarbon group may be saturated or unsaturated. Typically, the aliphatic hydrocarbon group is preferably saturated.

Examples of the aliphatic hydrocarbon group include a linear or branched aliphatic hydrocarbon group or an aliphatic hydrocarbon group having a ring in the structure thereof, and the like.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

The above-described linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Containing Ring in Structure Thereof

As the aliphatic hydrocarbon group including a ring in the structure thereof, a cyclic aliphatic hydrocarbon group which contains a hetero atom in a ring structure and may have a substituent (group obtained by removing two hydrogen atoms from an aliphatic hydrocarbon ring), a group in which the cyclic aliphatic hydrocarbon group is bonded to a terminal of a linear or branched aliphatic hydrocarbon group, a group in which the cyclic aliphatic hydrocarbon group is interposed in the middle of a linear or branched aliphatic hydrocarbon group, and the like are exemplary examples. Examples of the linear or branched aliphatic hydrocarbon group include the same ones as those described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group in which two hydrogen atoms have been removed from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and a methyl group, an ethyl group, a propyl group, an n-butyl group, or a tert-butyl group is more preferable.

The alkoxy group as the substituent is preferably an alkoxy group having 1 to 5 carbon atoms; more preferably a methoxy group, an ethoxy group, an n-propoxy group, an iso-propoxy group, an n-butoxy group, or a tert-butoxy group; and still more preferably a methoxy group or an ethoxy group.

The halogen atom as the substituent is preferably a fluorine atom.

Examples of the halogenated alkyl group as the substituent include groups in which a part or all of hydrogen atoms in the above-described alkyl groups have been substituted with the above-described halogen atoms.

In the cyclic aliphatic hydrocarbon group, some carbon atoms constituting the ring structure thereof may be substituted with a substituent having a hetero atom. As the substituent having a hetero atom, —O—, —C(═O)—O—, —S—, —S(═O)₂—, or —S(═O)₂—O— is preferable.

### Aromatic Hydrocarbon Group as Ya⁰

The aromatic hydrocarbon group is a hydrocarbon group having at least one aromatic ring.

The aromatic ring is not particularly limited as long as the aromatic ring has a cyclic conjugated system having (4n+2) pieces of π electrons, and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Provided that the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some of the carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group obtained by eliminating two hydrogen atoms from the above-described aromatic hydrocarbon ring or aromatic heterocyclic ring (an arylene group or a heteroarylene group); a group obtained by eliminating two hydrogen atoms from an aromatic compound having two or more aromatic rings (for example, biphenyl or fluorene); a group in which one hydrogen atom of a group obtained by eliminating one hydrogen atom from the above-described aromatic hydrocarbon ring or aromatic heterocyclic ring (an aryl group or a heteroaryl group) has been substituted with an alkylene group (for example, a group obtained by further eliminating one hydrogen atom from the aryl group in an arylalkyl group such as a benzyl group, a phenethyl group, a 1-naphthylmethyl group, a 2-naphthylmethyl group, a 1-naphthylethyl group, or a 2-naphthylethyl group); and the like. The alkylene group bonded to the aryl group or the heteroaryl group preferably has 1 to 4 carbon atoms, more preferably has 1 or 2 carbon atoms, and particularly preferably has 1 carbon atom.

In the above-described aromatic hydrocarbon group, a hydrogen atom in the aromatic hydrocarbon group may be substituted with a substituent. For example, the hydrogen atom bonded to the aromatic ring in the aromatic hydrocarbon group may be substituted with a substituent. Examples of the substituent include an alkyl group, an alkoxy group, a halogen atom, a halogenated alkyl group, and a hydroxyl group.

The alkyl group as the substituent is preferably an alkyl group having 1 to 5 carbon atoms, and a methyl group, an ethyl group, a propyl group, an n-butyl group, or a tert-butyl group is more preferable.

Examples of the alkoxy group, the halogen atom, and the halogenated alkyl group, as the substituent, include those exemplified as the substituent that is substituted for a hydrogen atom contained in the cyclic aliphatic hydrocarbon group.

### Divalent Linking Group Containing Hetero Atom:

In a case where Ya⁰ represents a divalent linking group having a hetero atom, preferred examples of the linking group include —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group, an acyl group, or the like), —S—, —S(═O)₂—, —S(═O)₂—O—, a group represented by General Formula: —Y⁰²¹—O—Y⁰²²—, —Y⁰²¹—O—, —Y⁰²¹—C(═O)—O—, —C(═O)—O—Y⁰²¹, —[Y⁰²¹—C(═O)—O]_{m″}—Y⁰²²—, —Y⁰²¹—C(═O)—Y⁰²²—, or —Y⁰²¹—S(═O)₂—O—Y⁰²¹— [In the formulae, Y⁰²¹ and Y⁰²² each independently represent a divalent hydrocarbon group which may have a substituent, 0 represents an oxygen atom, and m″ represents an integer in a range of 0 to 3], and the like.

In a case where the divalent linking group containing a hetero atom is —C(═O)—NH—, —C(═O)—NH—C(═O)—, —NH—, or —NH—C(═NH)—. H may be substituted with a substituent such as an alkyl group, an acyl group, or the like. The substituent (alkyl group, acyl group, and the like) preferably has 1 to 10 carbon atoms, more preferably has 1 to 8 carbon atoms, and particularly preferably has 1 to 5 carbon atoms. In General Formula —Y⁰²¹—O—Y⁰²²—, —Y⁰²¹—O—, —Y⁰²¹—C(═O)—O—, —C(═O)—O—Y⁰²¹—, —[Y⁰²¹—C(═O)—O]ₘ—Y⁰²²—, —Y⁰²¹—O—C(═O)—Y⁰²²—, or —Y⁰²¹—S(═O)₂—O—Y⁰²²—, Y⁰²¹ and Y⁰²² each independently represent a divalent hydrocarbon group which may have a substituent. Examples of the divalent hydrocarbon group include the same groups as those for the above-described divalent linking group (the divalent hydrocarbon groups which may have a substituent) as Ya⁰¹.

Y⁰²¹ represents preferably a linear aliphatic hydrocarbon group, more preferably a linear alkylene group, still more preferably a linear alkylene group having 1 to 5 carbon atoms, and particularly preferably a methylene group or an ethylene group.

Y⁰²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y⁰²¹—C(═O)—O]_{m″}—Y⁰²²—, m″ represents an integer of 0 to 3, preferably an integer of 0 to 2, more preferably 0 or 1, and particularly preferably 1. That is, a group represented by Formula —Y⁰²¹—C(═O)—O—Y⁰²²— is particularly preferable as the group represented by Formula —[Y⁰²¹—C(═O)—O]_{m″}—Y⁰²²—. Among these, a group represented by Formula —(CH₂)_{a′}—C(═O)—O—(CH₂)_{b′}— is preferable. In the formula, a′ represents an integer in a range of 1 to 10, preferably an integer in a range of 1 to 8, more preferably an integer in a range of 1 to 5, still more preferably 1 or 2, and most preferably 1. b′ is an integer in a range of 1 to 10, preferably an integer in a range of 1 to 8, more preferably an integer in a range of 1 to 5, still more preferably 1 or 2, and most preferably 1.

Among these, Ya⁰ is preferably a single bond or a linear or branched alkyl group, and more preferably a single bond.

In General Formula (a0), the fluorinated alkyl group as Ra and Ra² is preferably a linear or branched fluorinated alkyl group having 1 to 5 carbon atoms, and more preferably a trifluoromethyl group.

In General Formula (a0), at least one of Ra⁰¹ and Ra⁰², which are bonded to the carbon atom adjacent to SO₃, is preferably a fluorine atom from the viewpoint of the acid strength.

In General Formula (a0), n0 is preferably 1 to 3.

In General Formula (a0), M^{m+} represents an m-valent organic cation. Among these, a sulfonium cation and an iodonium cation are preferable.

m represents an integer of 1 or more.

Preferred examples of the cation moiety ((M^{m+})_{l/m}) include organic cations each represented by General Formulae (ca-1) to (ca-5).

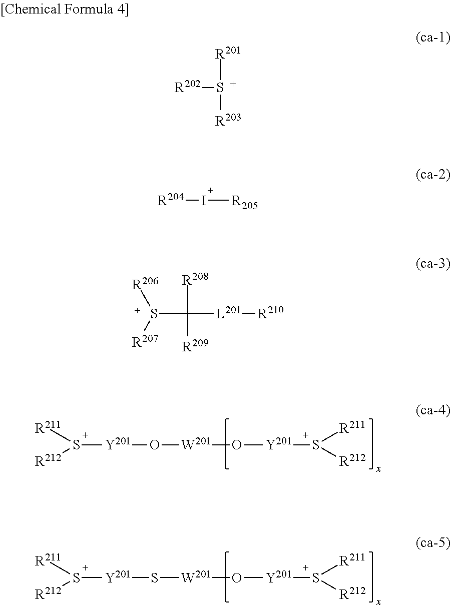

[In the formulae, R²⁰¹ to R²⁰⁷, R²¹¹, and R²¹² each independently represent an aryl group which may have a substituent, an alkyl group which may have a substituent, or an alkenyl group which may have a substituent. R²⁰¹ to R²⁰³, R²⁰⁶ and R²⁰⁷, and R²¹¹ and R²¹² may be bonded to each other to form a ring with the sulfur atom in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a SO₂-containing cyclic group which may have a substituent. L²⁰¹ represents —C(═O)— or —C(═O)—O—. Y²⁰¹'s each independently represent an arylene group, an alkylene group, or an alkenylene group. x represents 1 or 2. W²⁰¹ represents an (x+1)-valent linking group.]

In General Formulae (ca-1) to (ca-5), examples of the aryl group as R²⁰¹ to R²⁰⁷, R²¹¹, and R²¹² include an unsubstituted aryl group having 6 to 20 carbon atoms. Among these, a phenyl group or a naphthyl group is preferable.

The alkyl group as R²⁰¹ to R²⁰⁷, R²¹¹, and R²¹² is a chain-like or cyclic alkyl group, and the number of carbon atoms thereof is preferably in a range of 1 to 30.

It is preferable that the alkenyl group as R²⁰¹ to R²⁰⁷, R²¹¹, and R²¹² has 2 to 10 carbon atoms.

Examples of the substituent that R²⁰¹ to R²⁰⁷ and R²¹⁰ to R²¹² may have include an alkyl group, a halogen atom, a halogenated alkyl group, a carbonyl group, a cyano group, an amino group, an aryl group, and groups each represented by General Formulae (ca-r-1) to (ca-r-7).

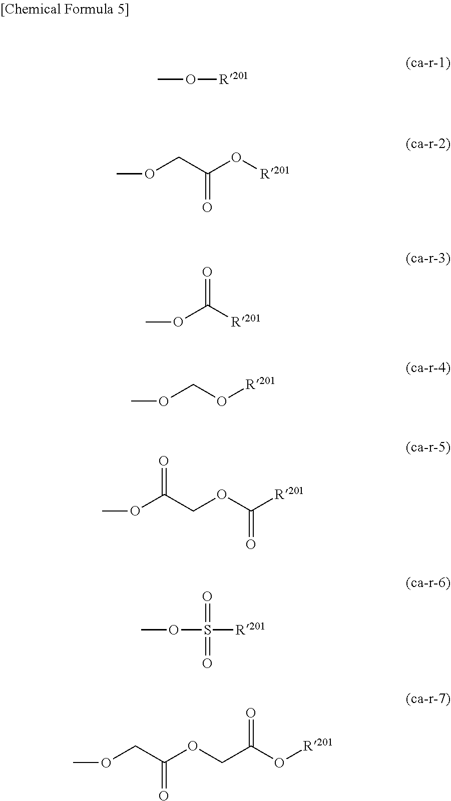

[In the formulae, R′²⁰¹'s each independently represent a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group with no aromaticity. In addition, the aliphatic hydrocarbon group may be saturated or unsaturated. Typically, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Provided that the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane has preferably 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms. Among these, a polycycloalkane having a crosslinked ring polycyclic skeleton such as adamantane, norbornane, isobornane, tricyclodecane, or tetracyclododecane; and a polycycloalkane having a condensed ring-based polycyclic skeleton such as a cyclic group having a steroid skeleton are preferable as the polycycloalkane.

Among these, the cyclic aliphatic hydrocarbon group as R′²⁰¹ is preferably a group obtained by removing one or more hydrogen atoms from a monocycloalkane or a polycycloalkane, more preferably a group obtained by removing one hydrogen atom from a polycycloalkane, particularly preferably an adamantyl group or a norbornyl group, and most preferably an adamantyl group.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, the cyclic hydrocarbon group as R′²⁰¹ may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and heterocyclic groups each represented by the other Chemical Formulae (r-hr-1) to (r-hr-16).

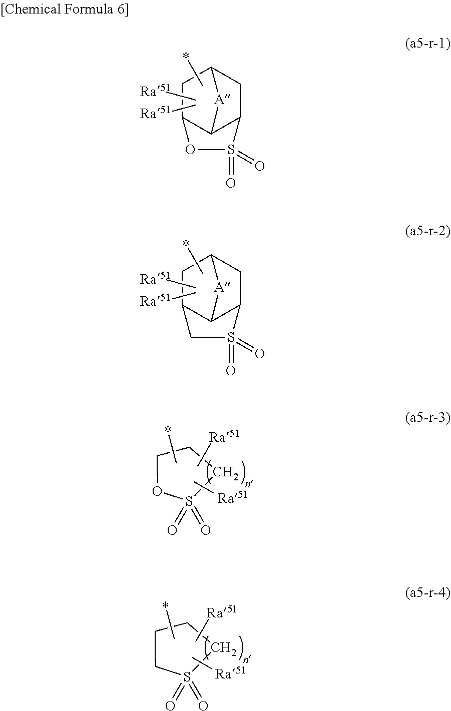

[In the formulae, Ra′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, a carbonate-containing cyclic group, or a —SO₂-containing cyclic group, A″ represents an alkylene group having 1 to 5 carbon atoms which may have an oxygen atom or a sulfur atom, an oxygen atom, or a sulfur atom, and n′ represents an integer of 0 to 2.]

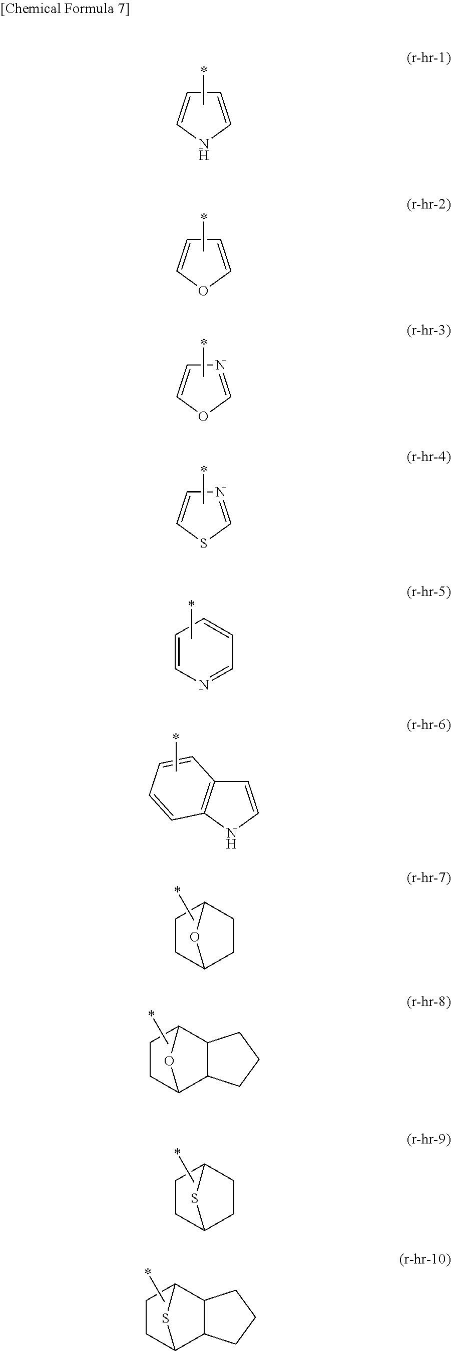

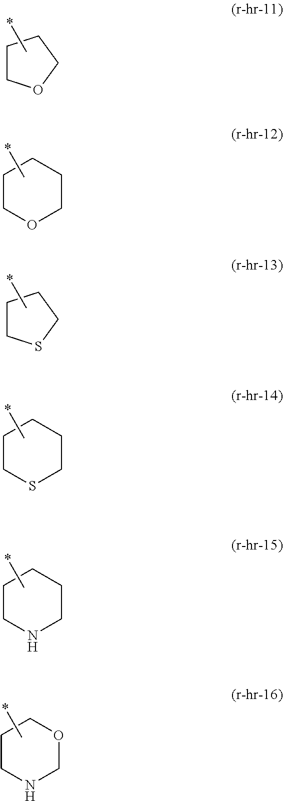

In General Formulae (a5-r-1) and (a5-r-2), A″ has the same definition as that for A″ in General Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′⁵¹ include the same groups as those for Ra′²¹ in General Formulae (a2-r-1) to (a2-r-7).

Specific examples of the groups each represented by General Formulae (a5-r-1) to (a5-r-4) are shown below. In the formulae shown below, “Ac” represents an acetyl group.

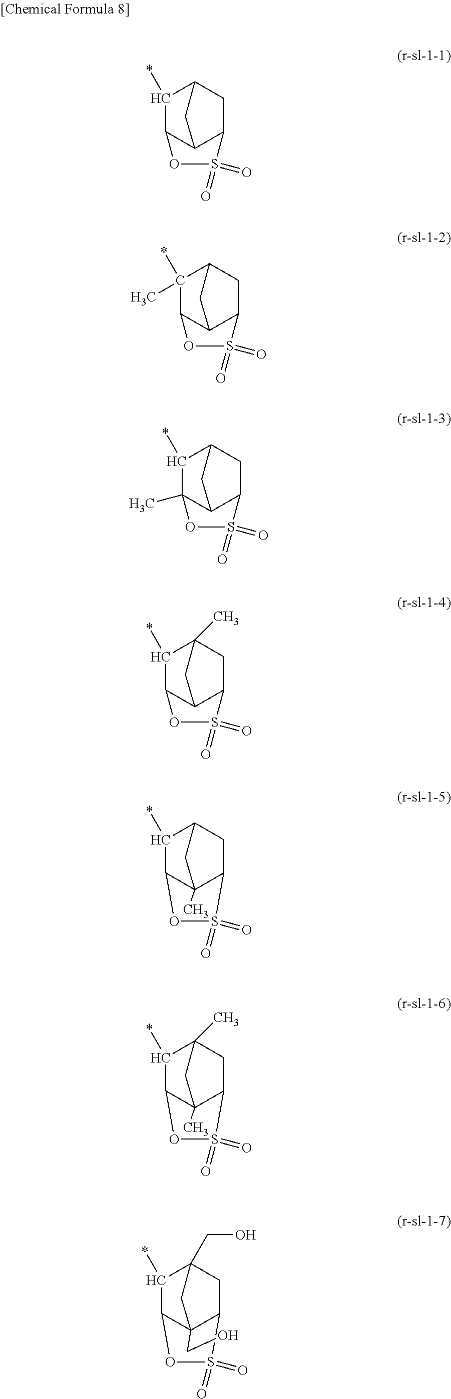

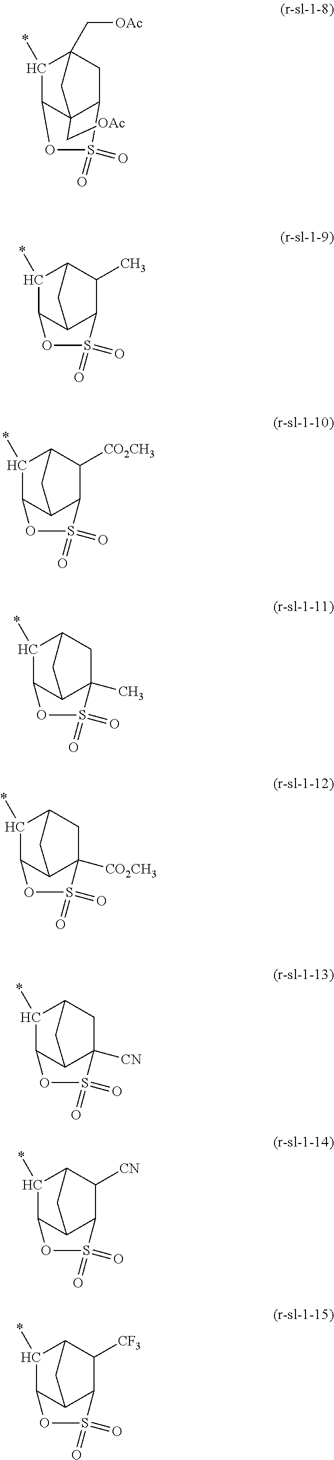

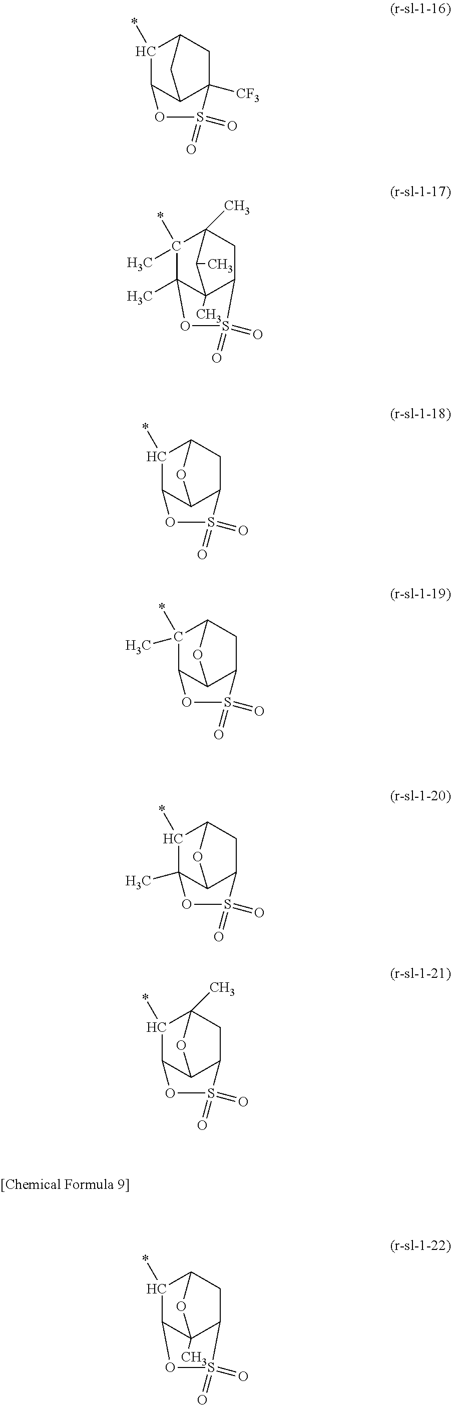

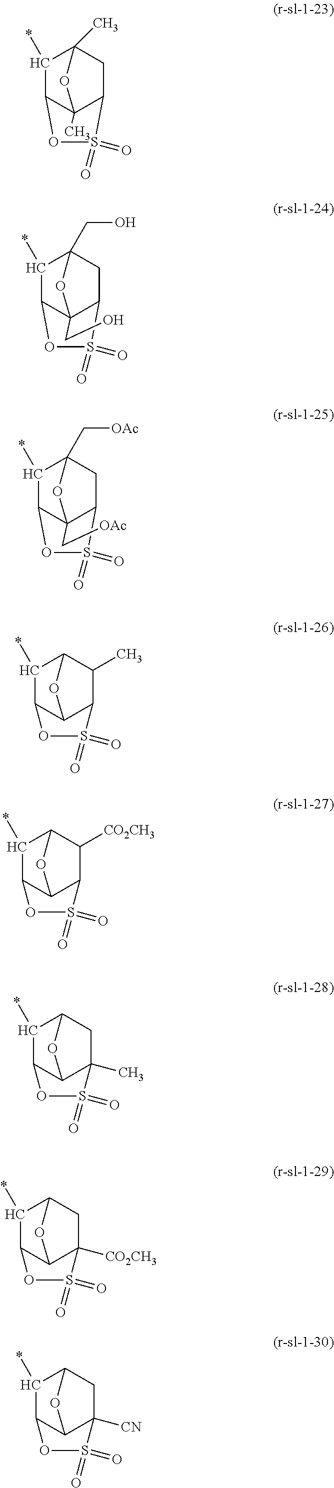

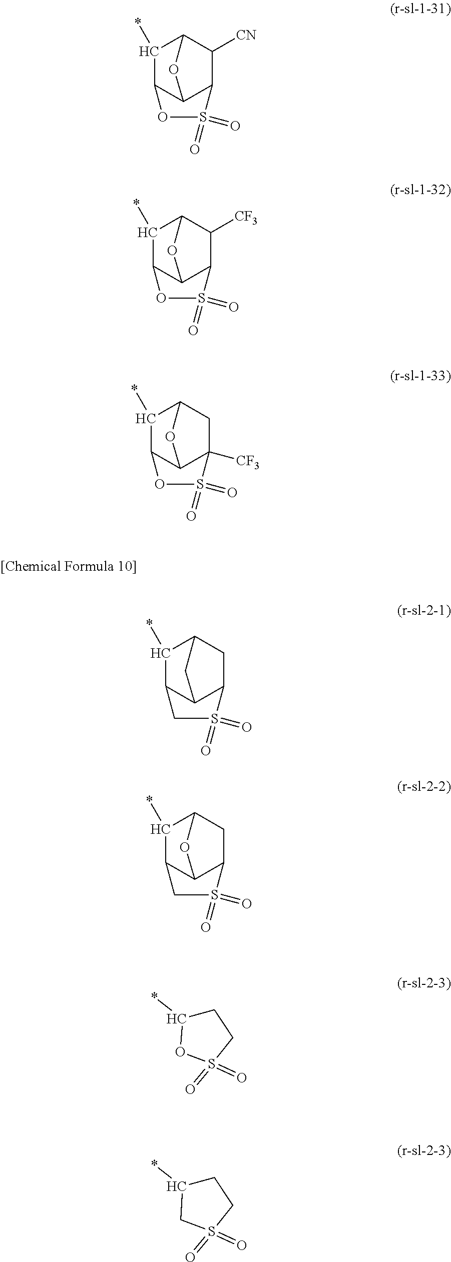

Examples of the substituent for the cyclic group as R′²⁰¹ include an alkyl group, an alkoxy group, a halogen atom, a halogenated alkyl group, a hydroxyl group, a carbonyl group, and a nitro group.

The alkyl group as the substituent is preferably an alkyl group having 1 to 5 carbon atoms, and a methyl group, an ethyl group, a propyl group, an n-butyl group, or a tert-butyl group is most preferable.

The alkoxy group as the substituent preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R′²⁰ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butenyl group. Examples of the branched alkenyl group include a 1-methylvinyl group, a 2-methylvinyl group, a 1-methylpropenyl group, and a 2-methylpropenyl group.

Among the above, the chain-like alkenyl group is preferably a linear alkenyl group, more preferably a vinyl group or a propenyl group, and particularly preferably a vinyl group.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, or the chain-like alkenyl group which may have a substituent as R′²⁰¹ include the same groups as those for the acid dissociable group represented by Formula (a1-r-2) as the cyclic group which may have a substituent or the chain-like alkyl group which may have a substituent, in addition to the groups described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (a5-r-1) to (a5-r-4).

In General Formulae (ca-1) to (ca-5), in a case where R²⁰¹ to R²⁰³, R²⁰⁶ and R²⁰⁷, and R²¹¹ and R²¹² are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂—, —SO₃—, —COO—, —CONH— or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a SO₂-containing cyclic group which may have a substituent.

Examples of the aryl group as R²¹⁰ include an unsubstituted aryl group having 6 to 20 carbon atoms. Among these, a phenyl group or a naphthyl group is preferable. As the alkyl group as R²¹⁰, a chain-like or cyclic alkyl group having 1 to 30 carbon atoms is preferable.

It is preferable that the alkenyl group as R²¹⁰ has 2 to 10 carbon atoms. As the SO₂-containing cyclic group as R²¹¹ which may have a substituent, “—SO₂-containing polycyclic group” is preferable, and a group represented by General Formula (a5-r-1) is more preferable.

Y²⁰¹'s each independently represent an arylene group, an alkylene group, or an alkenylene group.

Examples of the arylene group as Y²⁰¹ include a group in which one hydrogen atom has been removed from an aryl group exemplified as the aromatic hydrocarbon group represented by R¹⁰¹ in Formula (b-1).

Examples of the alkylene group and alkenylene group as Y²⁰¹ include a group in which one hydrogen atom has been removed from the group exemplified as the chain-like alkyl group or the chain-like alkenyl group as R¹⁰¹ in Formula (b-1).

In Formula (ca-4), x represents 1 or 2.

W²⁰¹ represents an (x+1)-valent linking group, that is, a divalent or trivalent linking group.

As the divalent linking group represented by W²⁰¹, a divalent hydrocarbon group which may have a substituent is preferable, and examples thereof include the same divalent hydrocarbon groups which may have a substituent as those for Ya²¹ in General Formula (a2-1). The divalent linking group as W²⁰¹ may be any of linear, branched, or cyclic and is preferably cyclic. Among these, a group in which two carbonyl groups are combined with both ends of the arylene group is preferable. Examples of the arylene group include a phenylene group and a naphthylene group. Among these, a phenylene group is particularly preferable.

Examples of the trivalent linking group as W²⁰¹ include a group in which one hydrogen atom has been removed from the above-described divalent linking group as W²⁰¹ and a group obtained by bonding the divalent linking group to another divalent linking group described above. As the trivalent linking group as W²⁰¹, a group obtained by bonding two carbonyl groups to an arylene group is preferable.

Specific examples of suitable cations represented by Formula (ca-1) include cations each represented by Chemical Formulae (ca-1-1) to (ca-1-70).

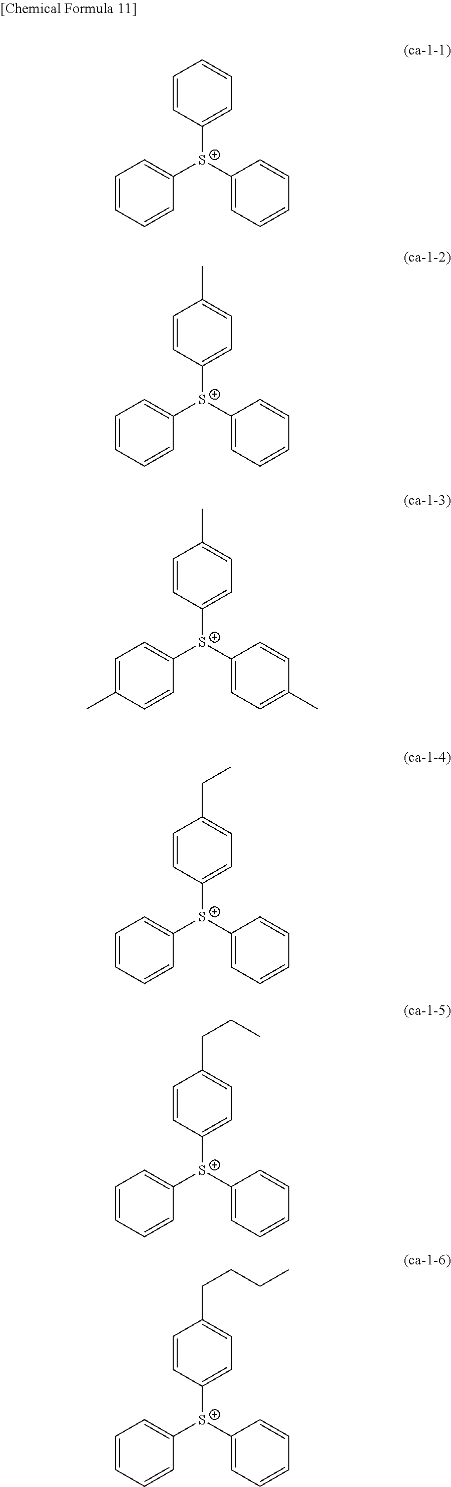

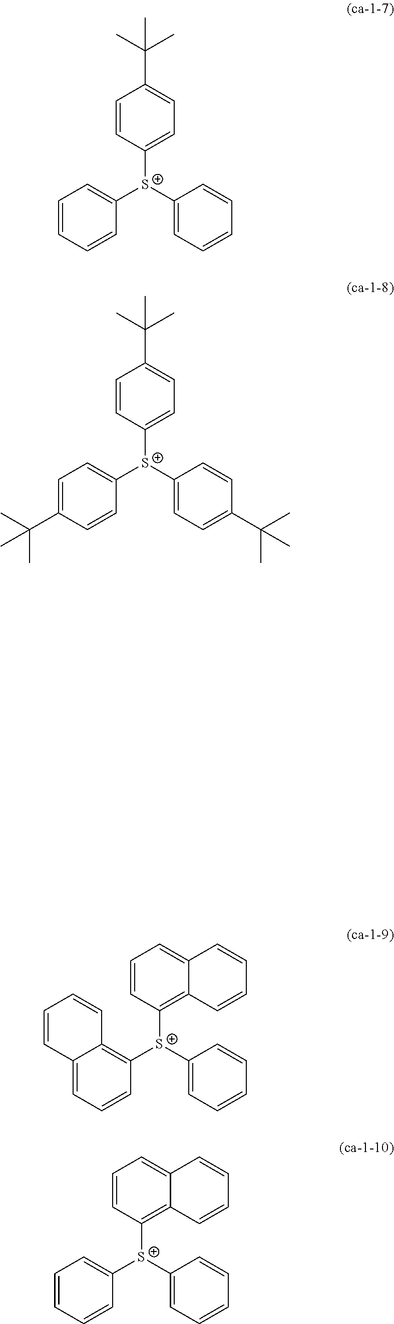 [SMILES: CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1]

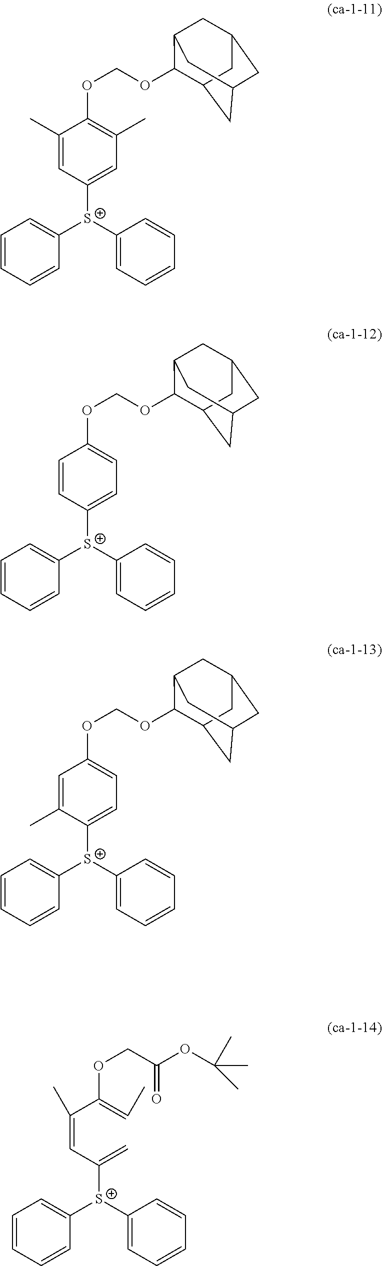 [SMILES: Cc1cc([S+](c2ccccc2)c2ccccc2)cc(C)c1OCOC1C2CC3CC(C2)CC1C3 ; c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; C=C(/C=C(C)\C(=C\C)OCC(=O)OC(C)(C)C)[S+](c1ccccc1)c1ccccc1]

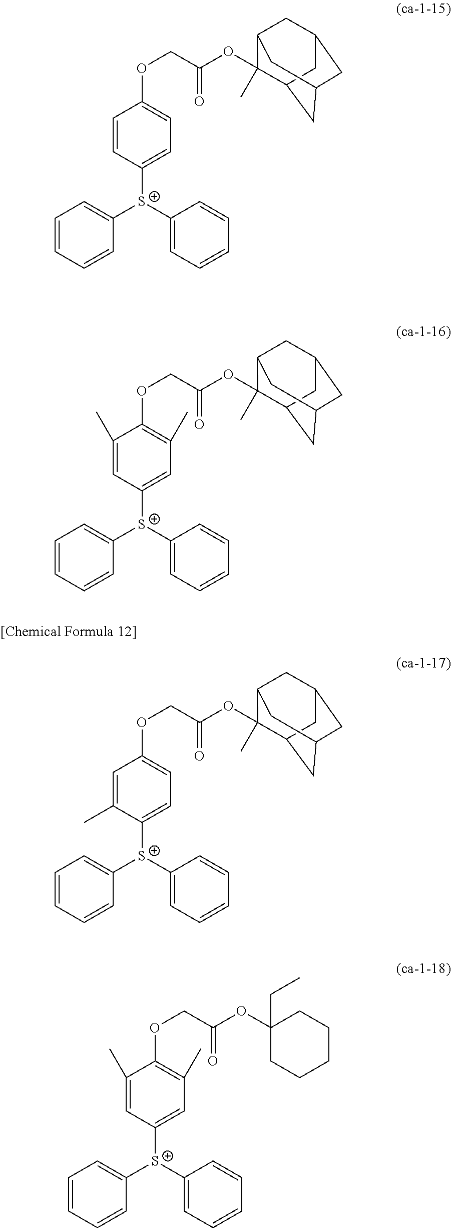 [SMILES: CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3 ; Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1]

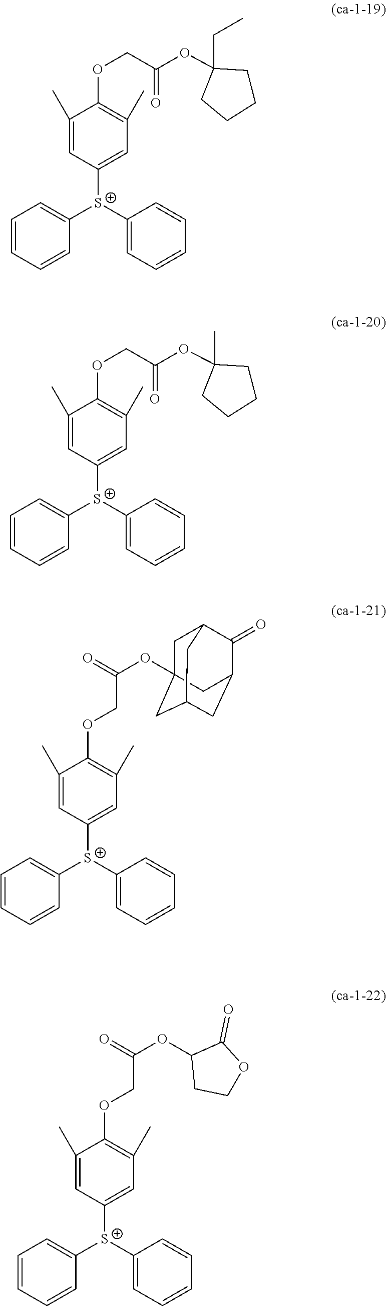 [SMILES: CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1 ; Cc1cc([S+](c2ccccc2)c2ccccc2)cc(C)c1OCC(=O)OC12CC3CC(C1)C(=O)C(C3)C2 ; Cc1cc([S+](c2ccccc2)c2ccccc2)cc(C)c1OCC(=O)OC1CCOC1=O]

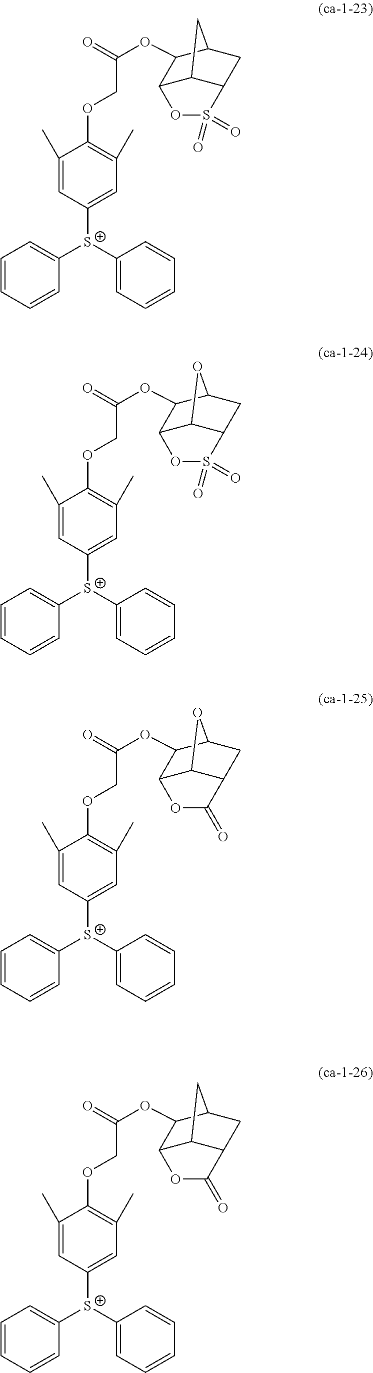 [SMILES: Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O ; Cc1cc([S+](c2ccccc2)c2ccccc2)cc(C)c1OCC(=O)OC1C2CC3C(=O)OC1C3O2 ; Cc1cc([S+](c2ccccc2)c2ccccc2)cc(C)c1OCC(=O)OC1C2CC3C(=O)OC1C3C2]

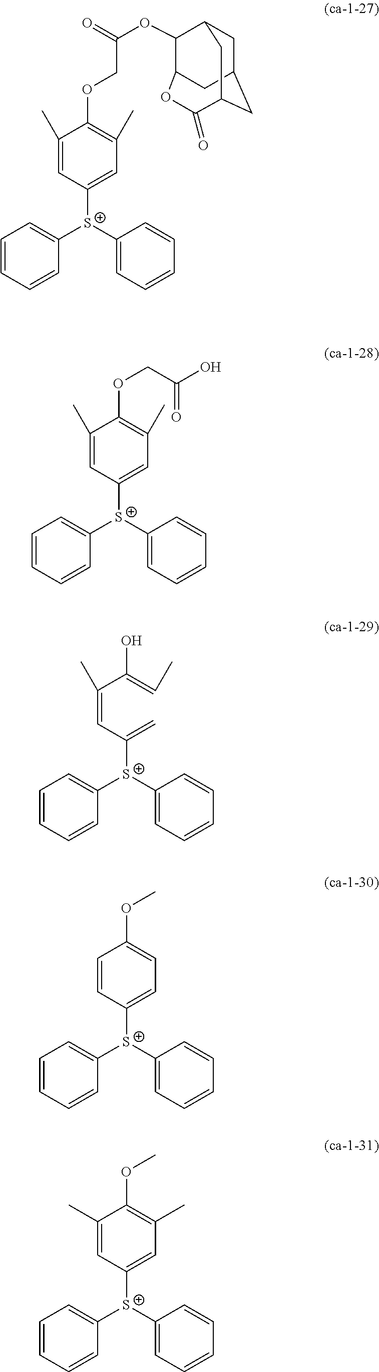 [SMILES: Cc1cc([S+](c2ccccc2)c2ccccc2)cc(C)c1OCC(=O)OC1C2CC3CC(C2)C(=O)OC1C3 ; Cc1cc([S+](c2ccccc2)c2ccccc2)cc(C)c1OCC(=O)O ; C=C(/C=C(C)\C(O)=C\C)[S+](c1ccccc1)c1ccccc1 ; COc1ccc([S+](c2ccccc2)c2ccccc2)cc1 ; COc1c(C)cc([S+](c2ccccc2)c2ccccc2)cc1C]

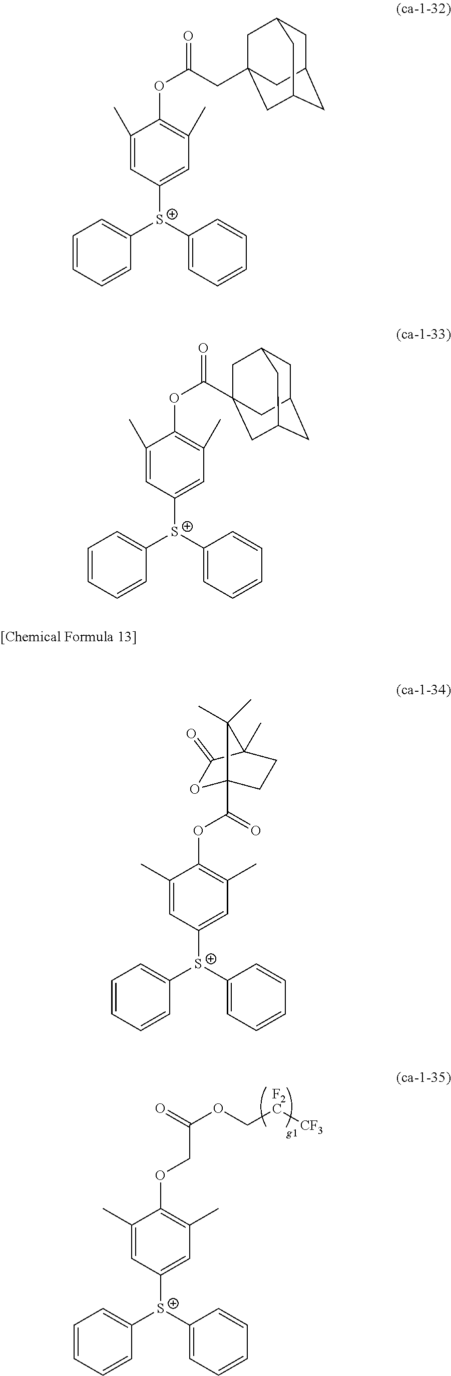

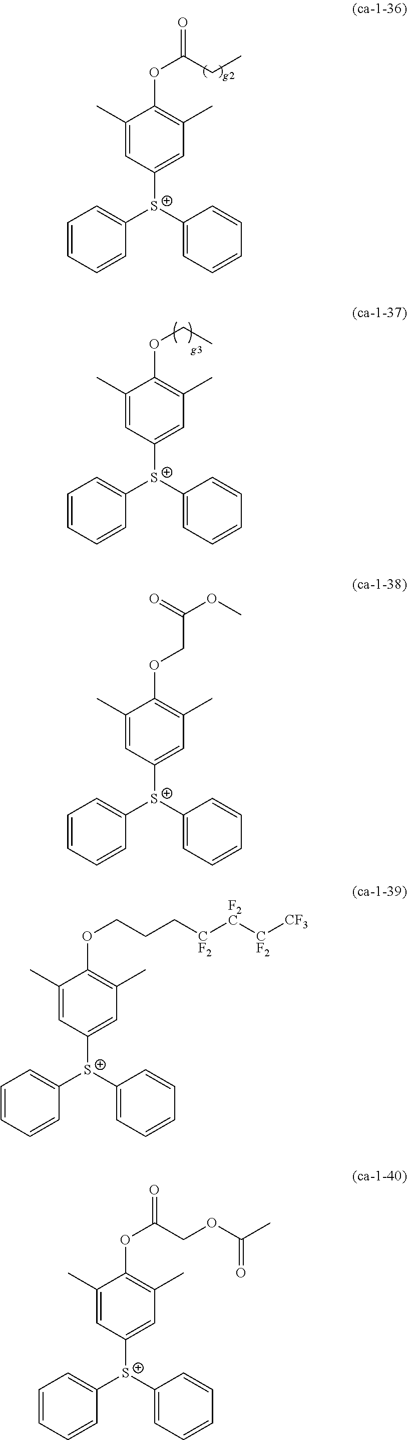

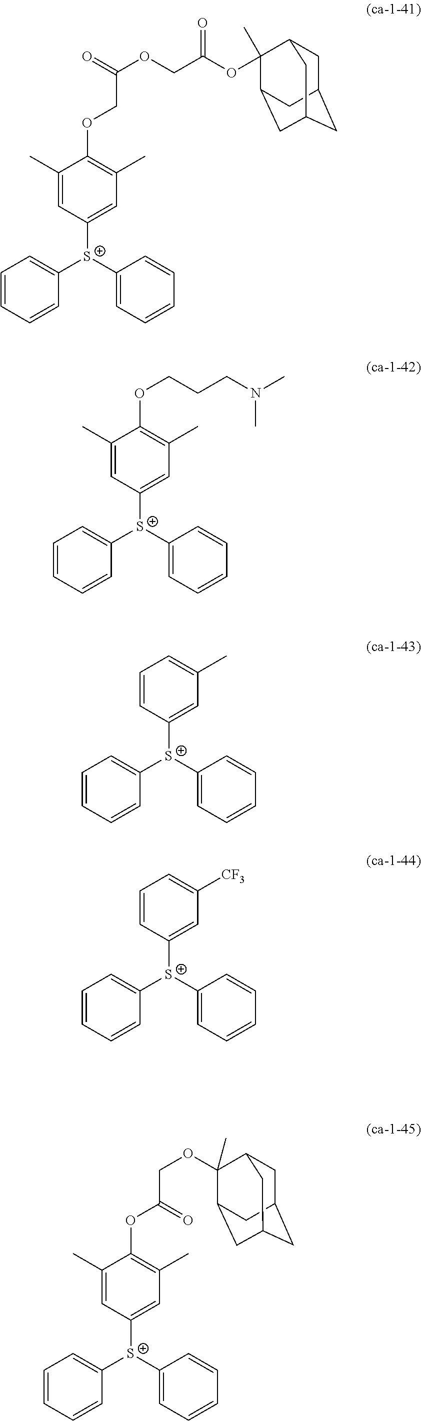 [SMILES: Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1 ; FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3]

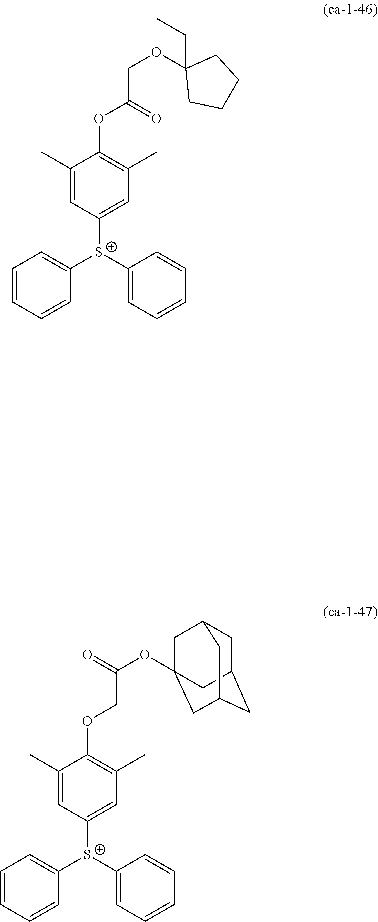 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

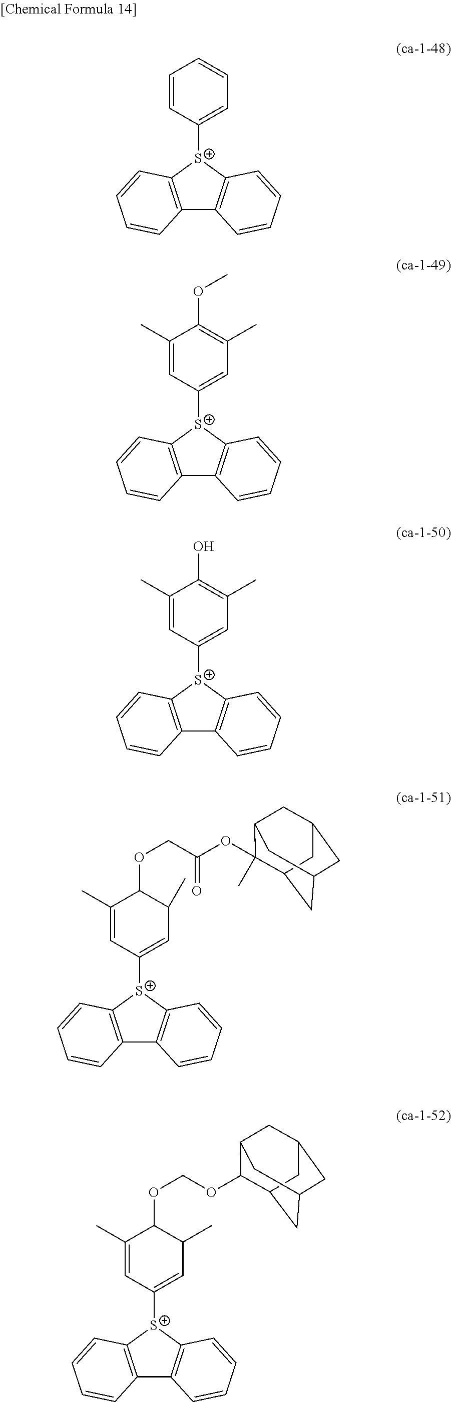 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3 ; CC1=CC([s+]2c3ccccc3c3ccccc32)=CC(C)C1OCOC1C2CC3CC(C2)CC1C3]

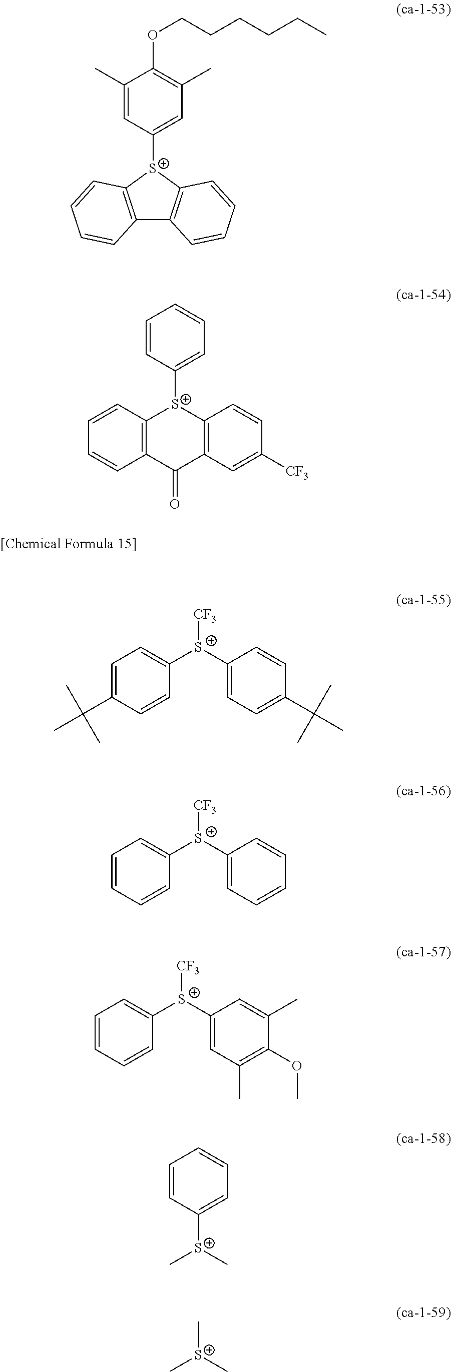

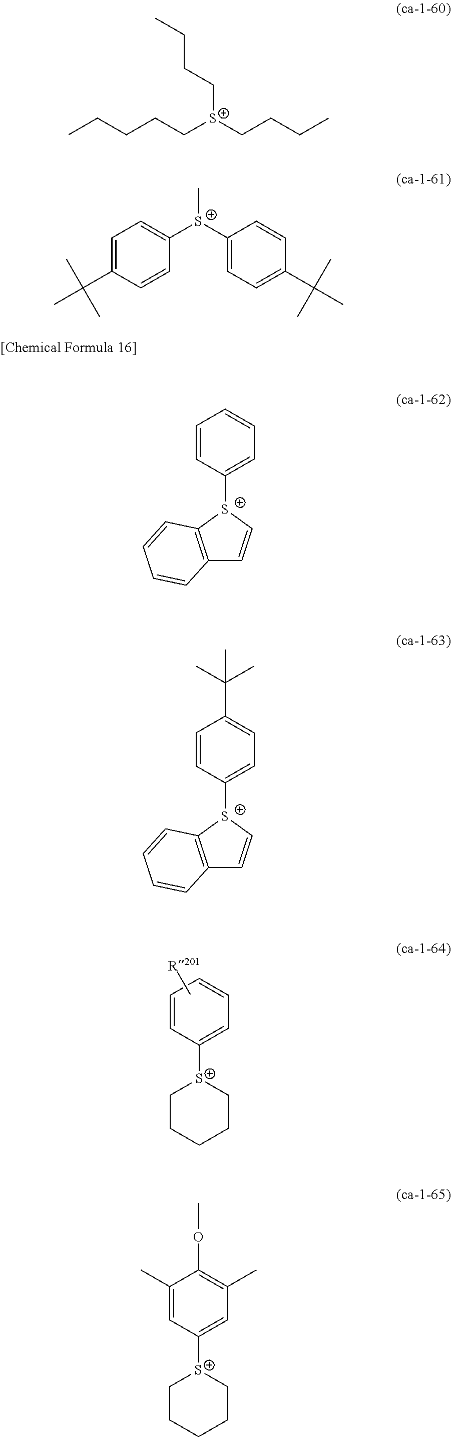

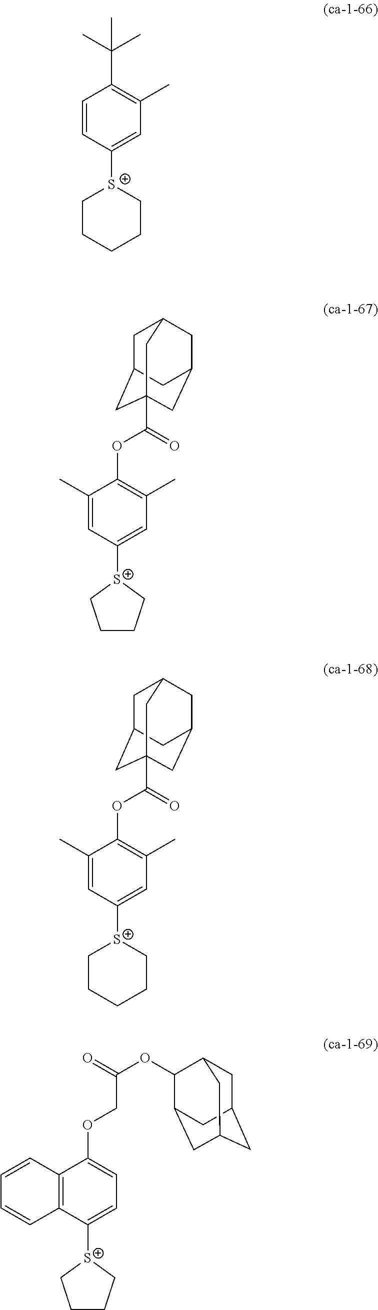 [SMILES: Cc1cc([S+]2CCCCC2)ccc1C(C)(C)C ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

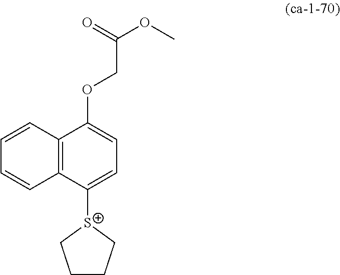 [SMILES: COC(=O)COc1ccc([S+]2CCCC2)c2ccccc12]

[In the formulae, R″²⁰¹ represents a hydrogen atom or a substituent, and examples of the substituent include the same groups as those for the substituents which may be contained in R²⁰¹ to R²⁰⁷ and R²¹⁰ to R²¹².]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

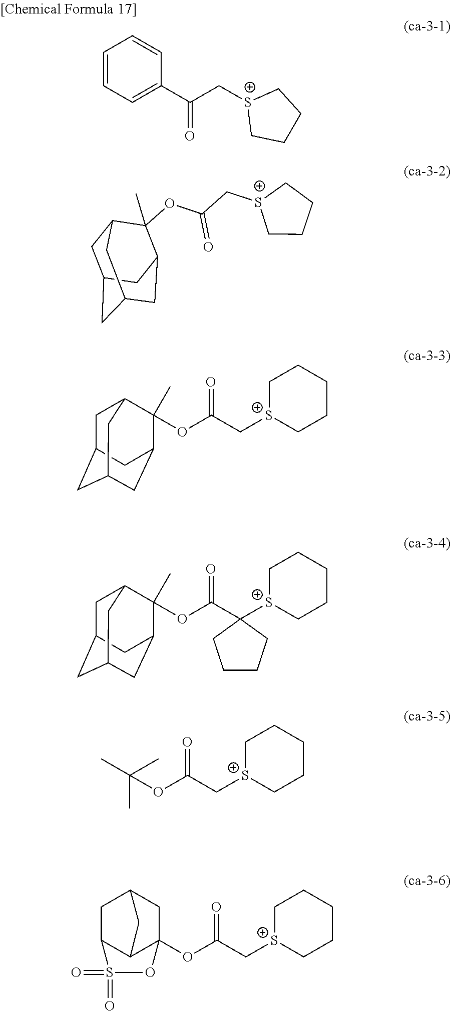 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC12CC3CC1C(C3)S(=O)(=O)O2]

Specific examples of suitable cations represented by Formula (ca-4) include cations each represented by Formulae (ca-4-1) and (ca-4-2).

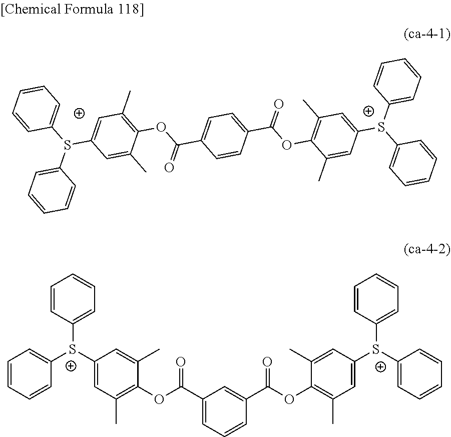

Specific examples of suitable cations represented by Formula (ca-5) include cations each represented by General Formulae (ca-5-1) and (ca-5-3).

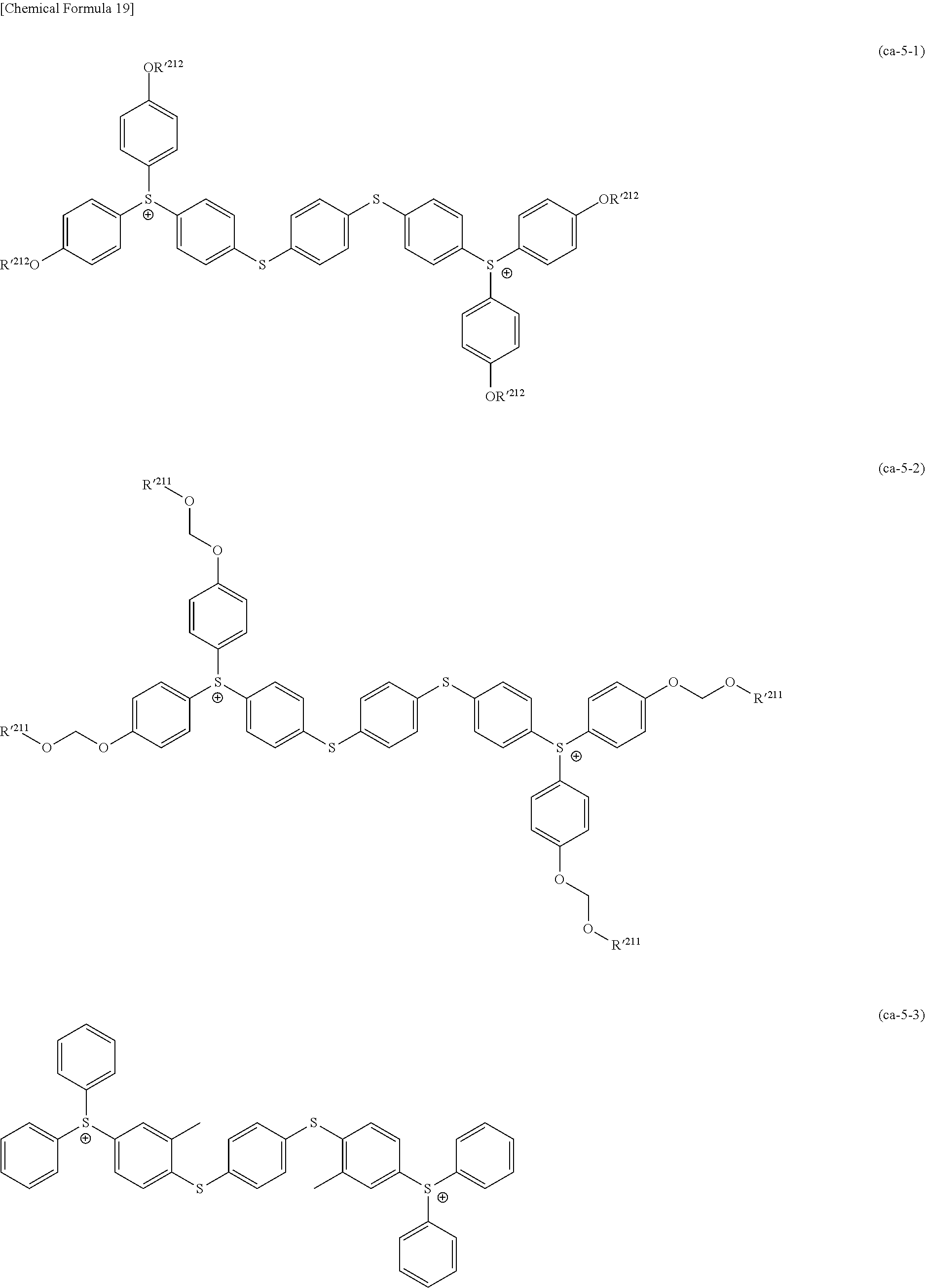

Among the examples, a cation represented by General Formula (ca-1) is preferable as the cation moiety ((M^{m+})_{l/m}). Among these, from the viewpoint of increasing the sensitivity, a cation represented by General Formula (ca-1), in which the aryl group, the alkyl group, or the alkenyl group as R²⁰¹ to R²⁰³ has a fluorine atom or a fluorinated alkyl group as a substituent, is more preferable.

Preferred specific examples of the cation represented by General Formula (ca-1) are shown below.

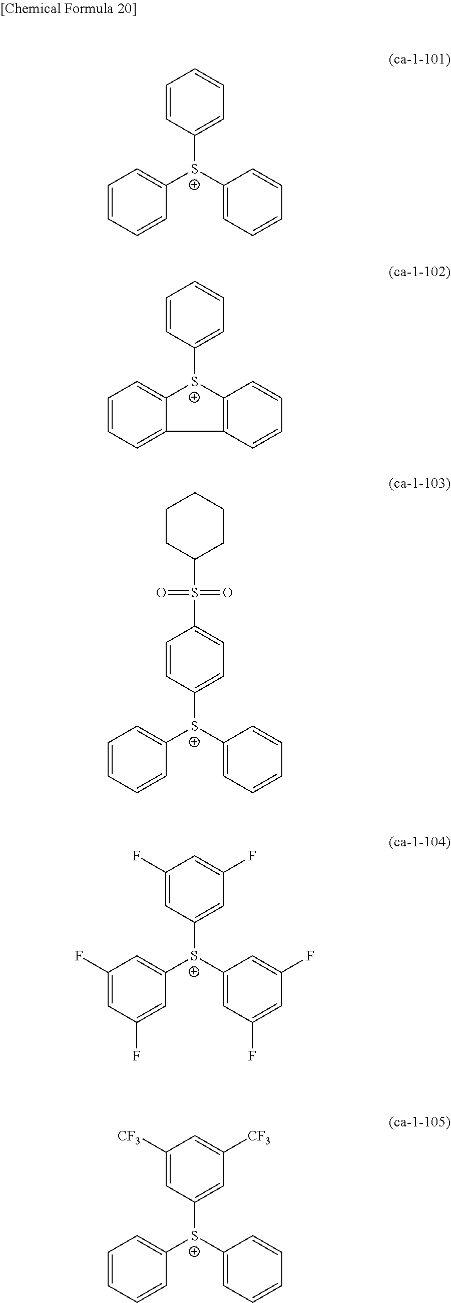

The constitutional unit (a0) is preferably a constitutional unit derived from a compound represented by General Formula (a0-1).

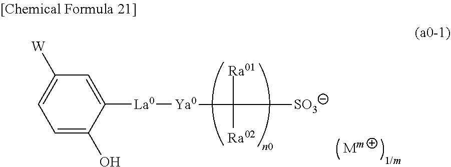

[In the formula, W is a polymerizable group. La⁰¹ represents —C(═O)—O— or —O—C(═O)—. Ya⁰ represents a single bond or a linear or branched alkylene group. Ra⁰¹ and Ra⁰² each independently represent a hydrogen atom, a fluorine atom, or a fluorinated alkyl group. n0 represents an integer in a range of 1 to 4. m represents an integer of 1 or more, and M^{m+} represents an m-valent organic cation.]

In General Formula (a0-1), W, Ya⁰, Ra⁰¹, Ra⁰², n0, m, and M^{m+} are the same as W, Ya⁰, Ra⁰¹, Ra⁰², n0, m, and M^{m+} in General Formula (a0).

Preferred specific examples of the constitutional unit (a0) are shown below. In the formula, R⁰ represents a hydrogen atom, an alkyl group having 1 to 5 carbon atoms, a halogen atom, or a halogenated alkyl group having 1 to 5 carbon atoms, and is preferably a hydrogen atom. m and M^{m+} are the same as m and M^{m+} in General Formula (a0).

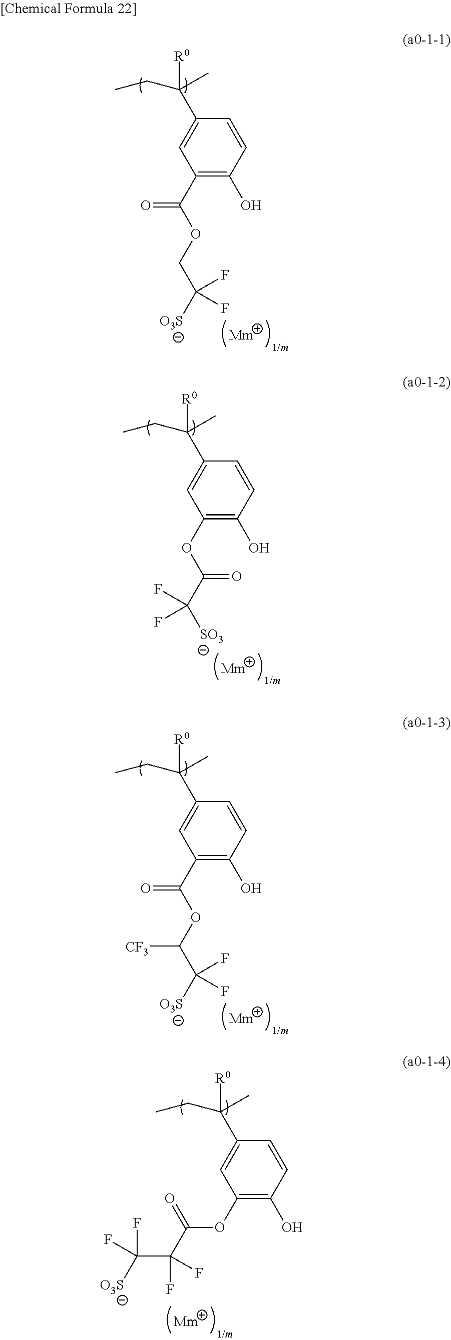

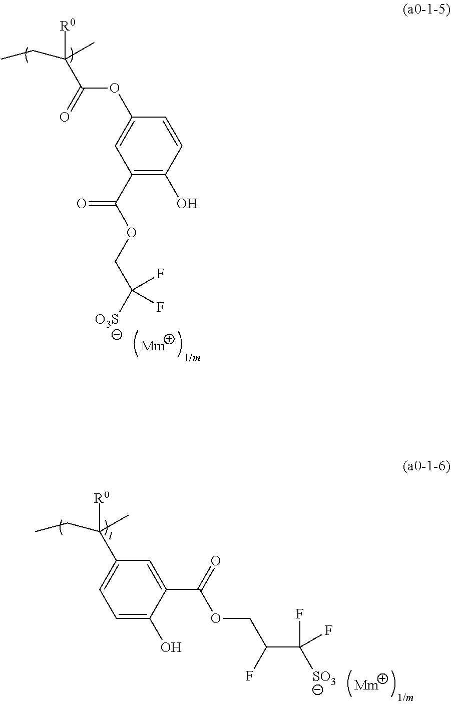

Among the above, the constitutional unit (a0) is preferably a constitutional unit represented by any one of General Formulae (a0-1-1) to (a0-1-6).

The constitutional unit (a0) contained in the component (A1) may be one kind or may be two or more kinds.

The proportion of the constitutional unit (a0) is preferably 1% to 50% by mole, more preferably 5% to 40% by mole, still more preferably 10% to 30% by mole, and particularly preferably 15% to 25% by mole, with respect to the total amount (100% by mole) of all the constitutional units constituting the component (A1).

In a case where the proportion of the constitutional unit (a0) is equal to or more than the lower limit value of the above-described preferred range, the sensitivity of the resist composition is easily enhanced. On the other hand, in a case where the proportion of the constitutional unit (a0) is equal to or less than the preferred lower limit value described above, the lithography characteristics such as LWR are easily to be improved.

<<Constitutional unit (a1)>>

The constitutional unit (a1) is a constitutional unit containing an acid decomposable group whose polarity is increased under action of an acid.

Examples of the acid dissociable group are the same as those which have been suggested as the acid dissociable groups of the base resin for a chemically amplified resist composition.

Specific examples of the acid dissociable group of the base resin proposed for a chemically amplified resist composition include an “acetal type acid dissociable group”, a “tertiary alkyl ester type acid dissociable group”, and a “tertiary alkyloxycarbonyl acid dissociable group”, which are described later.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

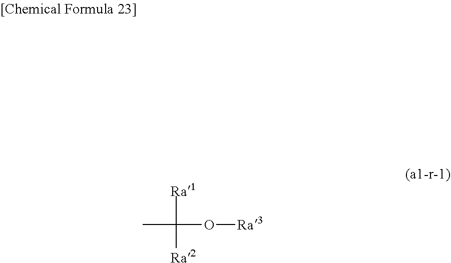

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ or Ra′² represents a hydrogen atom and more preferable that both Ra′¹ and Ra′² represent a hydrogen atom. In a case where Ra′¹ or Ra′² represents an alkyl group, examples of the alkyl group include the same alkyl groups exemplified as the substituent which may be bonded to the carbon atom at the α-position in the description of the α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

The aliphatic hydrocarbon group which is a monocyclic group is preferably a group obtained by removing one hydrogen atom from a monocycloalkane. The monocycloalkane has preferably 3 to 6 carbon atoms, and specific examples thereof include cyclopentane and cyclohexane.

The aliphatic hydrocarbon group which is a polycyclic group is preferably a group obtained by removing one hydrogen atom from a polycycloalkane. The polycycloalkane preferably has 7 to 12 carbon atoms, and specific examples thereof include adamantane, norbornane, isobornane, tricyclodecane, tetracyclododecane, and the like.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some of the carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aromatic hydrocarbon ring or aromatic heterocyclic ring preferably has 1 to 4 carbon atoms, more preferably has 1 or 2 carbon atoms, and particularly preferably has 1 carbon atom.

The cyclic hydrocarbon group as Ra′³ may have a substituent. Examples of the substituent include —R^{P1}, —R^{P2}—O—R^{P1}, —R^{P2}—CO—R^{P1}, —R^{P2}—CO—OR^{P}, —R^{P2}—O—CO—R^{P1}, —R^{P2}—OH, —R^{P2}—CN, and —R^{P2}—COOH (hereinafter, these substituents will also be collectively referred to as “Ra^{x5}”).

Here, R^{P1} represents a monovalent chain-like saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. In addition, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of the single kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7] dodecanyl group, or an damantly group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2).

Among the acid dissociable groups represented by General Formula (a1-r-2), for convenience, a group which is constituted of alkyl groups is referred to as a “tertiary alkyl ester type acid dissociable group”.

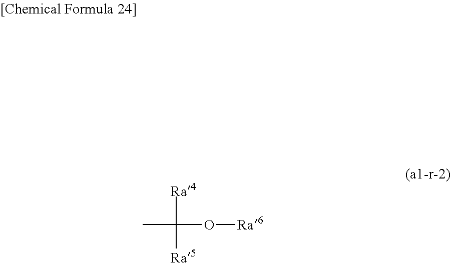

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

The chain-like or cyclic alkenyl group as Ra′⁴ is preferably an alkenyl group having 2 to 10 carbon atoms.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

On the other hand, in a case where Ra′⁴ to Ra′⁶ independently represent a hydrocarbon group without being bonded to one another, suitable examples thereof include a group represented by General Formula (a1-r2-4).

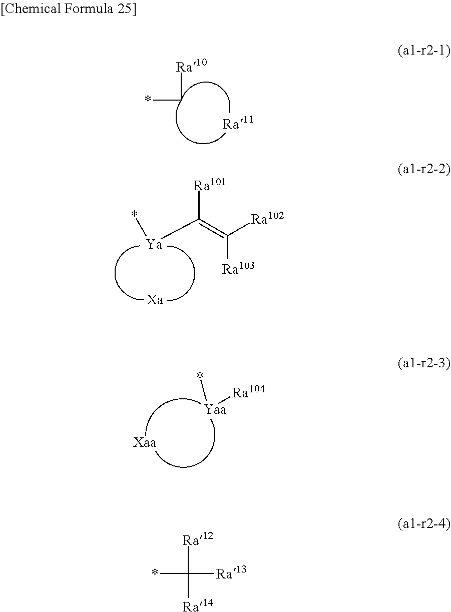

[In Formula (a1-r2-1), Ra′¹⁰ represents a linear or branched alkyl group having 1 to 12 carbon atoms, in which a part thereof may be substituted with a halogen atom or a hetero atom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group together with Ya. Some or all hydrogen atoms in this cyclic hydrocarbon group may be substituted. Ra¹⁰¹ to Ra¹⁰³ each independently represent a hydrogen atom, a monovalent chain-like saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a monovalent chain-like saturated hydrocarbon group having 1 to 10 carbon atoms. Some or all hydrogen atoms in this chain-like saturated hydrocarbon group may be substituted. Ra′¹⁴ represents a hydrocarbon group which may have a substituent. * represents a bonding site.]

In Formula (a1-r2-1), Ra′¹⁰ represents a linear or branched alkyl group having 1 to 12 carbon atoms, in which a part thereof may be substituted with a halogen atom or a hetero atom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a hetero atom-containing group. For example, some hydrogen atoms constituting the alkyl group may be substituted with a halogen atom or a hetero atom-containing group. In addition, some carbon atoms (methylene group or the like) constituting the alkyl group may be substituted with a hetero atom-containing group.

Examples of the hetero atoms here include an oxygen atom, a nitrogen atom, and a sulfur atom. Examples of the hetero atom-containing group include (—O—), —C(═O)—O—, —O—C(═O)—, —C(═O)—, —O—C(═O)—O—, —C(═O)—NH—, —NH—, —S—, —S(═O)₂—, —S(═O)₂—O—, and the like.

In Formula (a1-r2-1), preferred examples of Ra′¹¹ (an aliphatic cyclic group that is formed together with a carbon atom to which Ra′¹⁰ is bonded) include the groups exemplified as the aliphatic hydrocarbon group (alicyclic hydrocarbon group) which is a monocyclic group or a polycyclic group as Ra′³ in Formula (a1-r-1). Among them, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (aliphatic hydrocarbon group) as Ra′³ in Formula (a1-r-1).

The cyclic hydrocarbon group that is formed by Xa together with Ya may have a substituent. Examples of the substituent include those exemplified as the substituents which may be contained in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2] octanyl group, a tricyclo[5.2.1.02,6]decanyl group, a tricyclo[3.3.1.13,7]decanyl group, a tetracyclo[6.2.1.13,6.02,7] dodecanyl group, or an damantly group.

From the viewpoint of ease of synthesis. Ra¹⁰¹ to Ra¹⁰³ are preferably a hydrogen atom or a monovalent chain-like saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ include the same groups as those for Ra^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group exemplified as the aliphatic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group obtained by removing one or more hydrogen atoms from an aromatic hydrocarbon ring having 6 to 15 carbon atoms, more preferably a group obtained by removing one or more hydrogen atoms from benzene, naphthalene, anthracene, or phenanthrene, still more preferably a group obtained by removing one or more hydrogen atoms from benzene, naphthalene, or anthracene, particularly preferably a group obtained by removing one or more hydrogen atoms from benzene or naphthalene, and most preferably a group obtained by removing one or more hydrogen atoms from benzene.

Examples of the substituent that Ra¹⁰⁴ in Formula (a1-r2-3) may have include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a monovalent chain-like saturated hydrocarbon group having 1 to 10 carbon atoms. Examples of the monovalent chain-like saturated hydrocarbon group having 1 to 10 carbon atoms as Ra′¹² and Ra′¹³ include those exemplified as the monovalent chain-like saturated hydrocarbon group having 1 to 10 carbon atoms as Ra¹⁰¹ to Ra¹⁰³. Some or all hydrogen atoms in this chain-like saturated hydrocarbon group may be substituted.

Among them, Ra′¹² and Ra′¹³ are preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent thereof include the same groups as those for Ra^{x5}.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

The aliphatic hydrocarbon group which is a monocyclic group is preferably a group obtained by removing one hydrogen atom from a monocycloalkane. The monocycloalkane has preferably 3 to 6 carbon atoms, and specific examples thereof include cyclopentane and cyclohexane.

The aliphatic hydrocarbon group which is a polycyclic group is preferably a group obtained by removing one hydrogen atom from a polycycloalkane. The polycycloalkane preferably has 7 to 12 carbon atoms, and specific examples thereof include adamantane, norbornane, isobornane, tricyclodecane, tetracyclododecane, and the like.

Examples of the aromatic hydrocarbon group as Ra′¹⁴ include the same groups as those for the aromatic hydrocarbon group as Ra′¹⁰⁴. Among these, Ra′¹⁴ represents preferably a group obtained by removing one or more hydrogen atoms from an aromatic hydrocarbon ring having 6 to 15 carbon atoms, more preferably a group obtained by removing one or more hydrogen atoms from benzene, naphthalene, anthracene, or phenanthrene, still more preferably a group obtained by removing one or more hydrogen atoms from benzene, naphthalene, or anthracene, particularly preferably a group obtained by removing one or more hydrogen atoms from naphthalene or anthracene, and most preferably a group obtained by removing one or more hydrogen atoms from naphthalene.

Examples of the substituent which may be contained in Ra′¹⁴ include the same groups as those for the substituent which may be contained in Ra¹⁰⁴.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

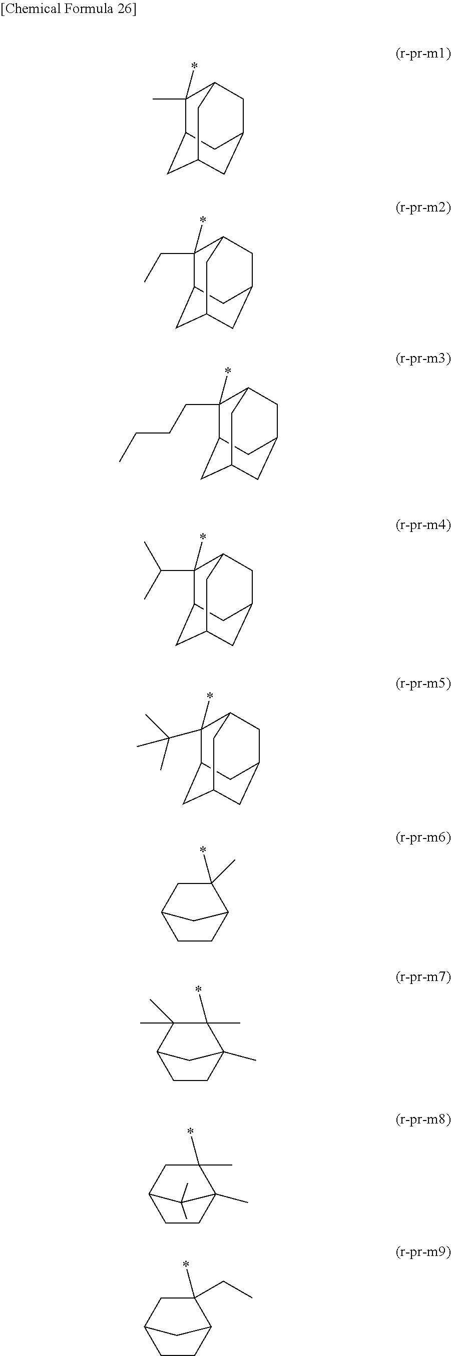

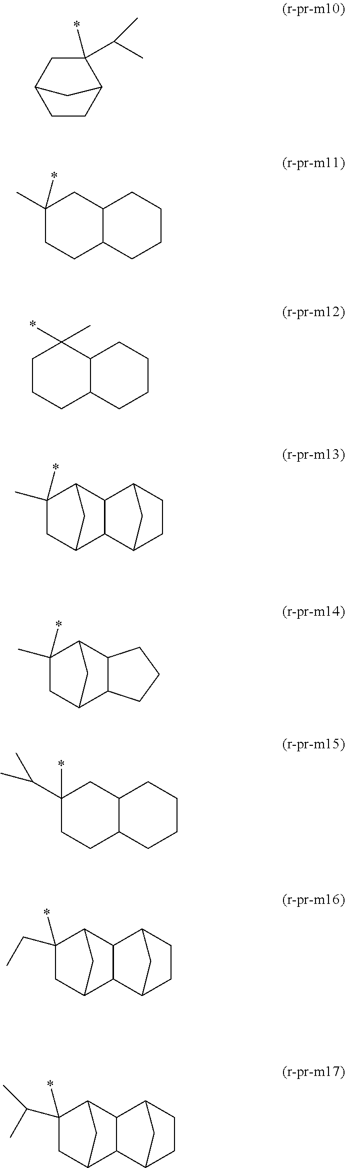

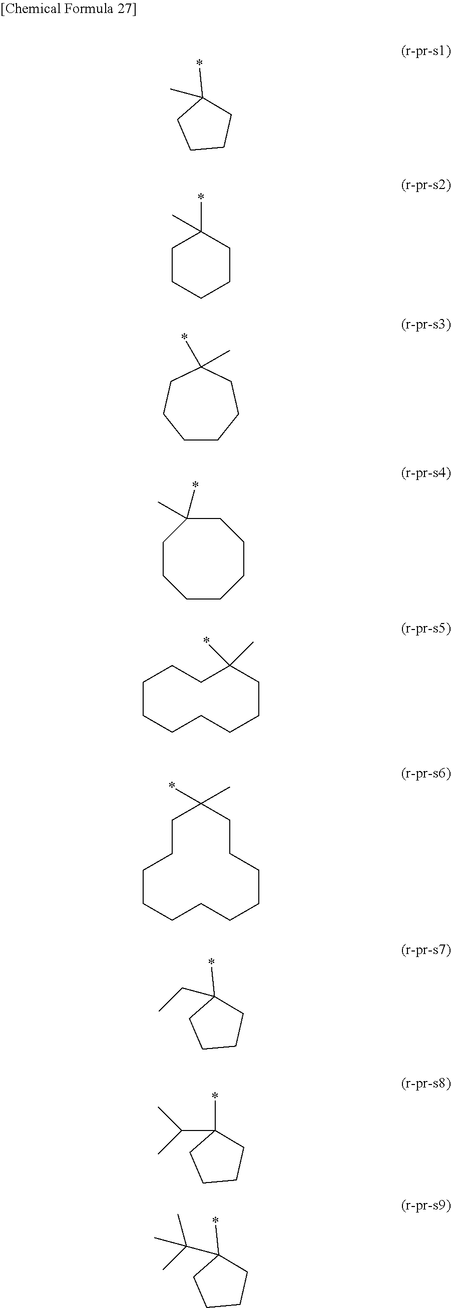

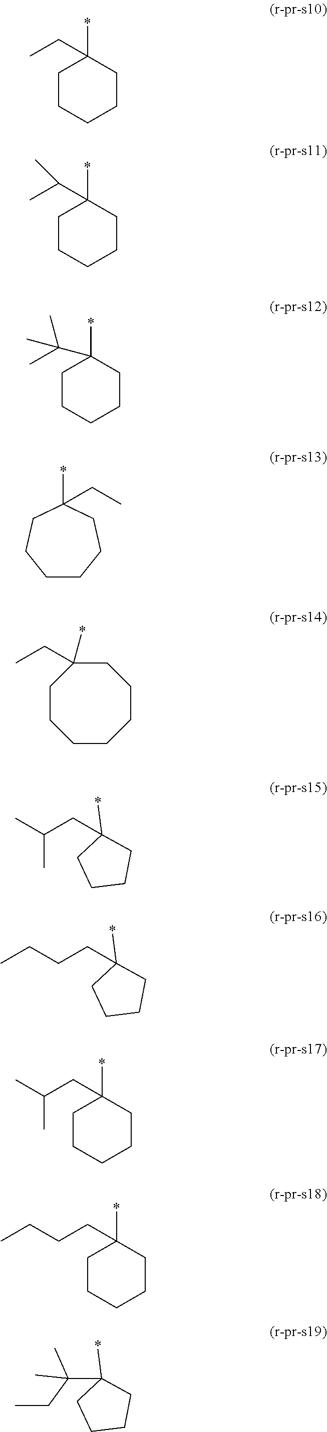

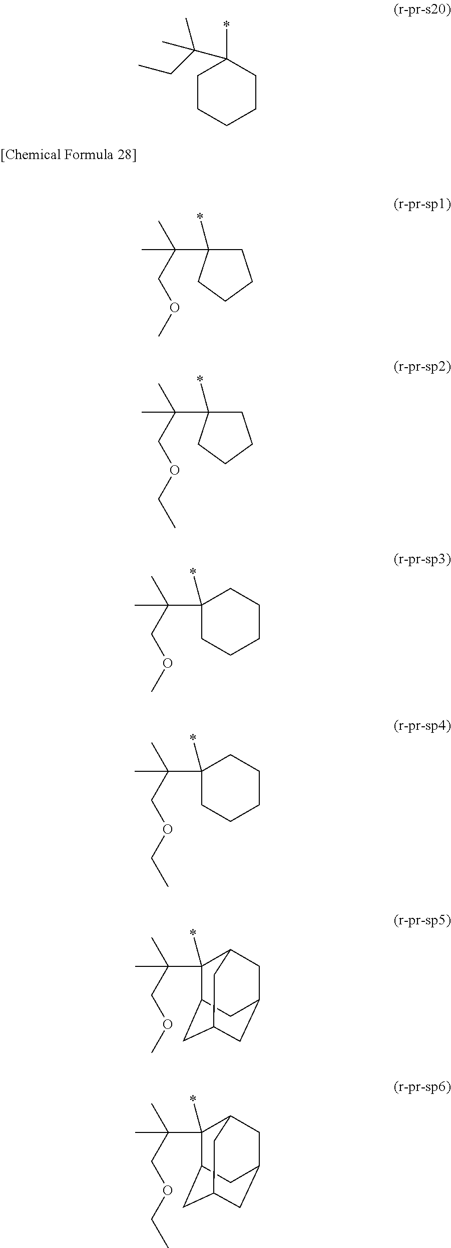

Specific examples of the group represented by Formula (a1-r2-2) are shown below.

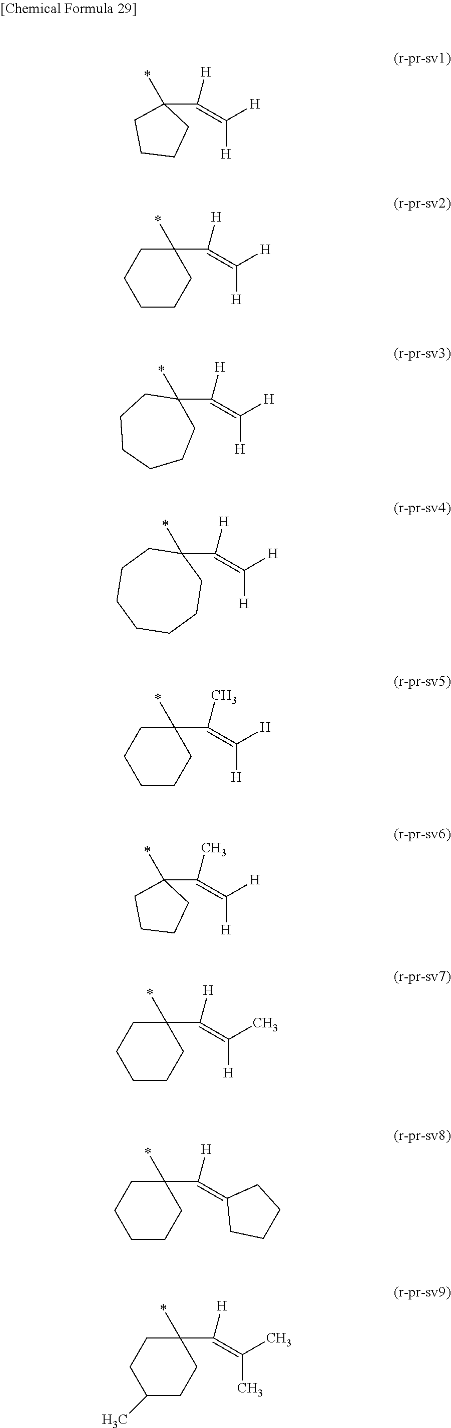

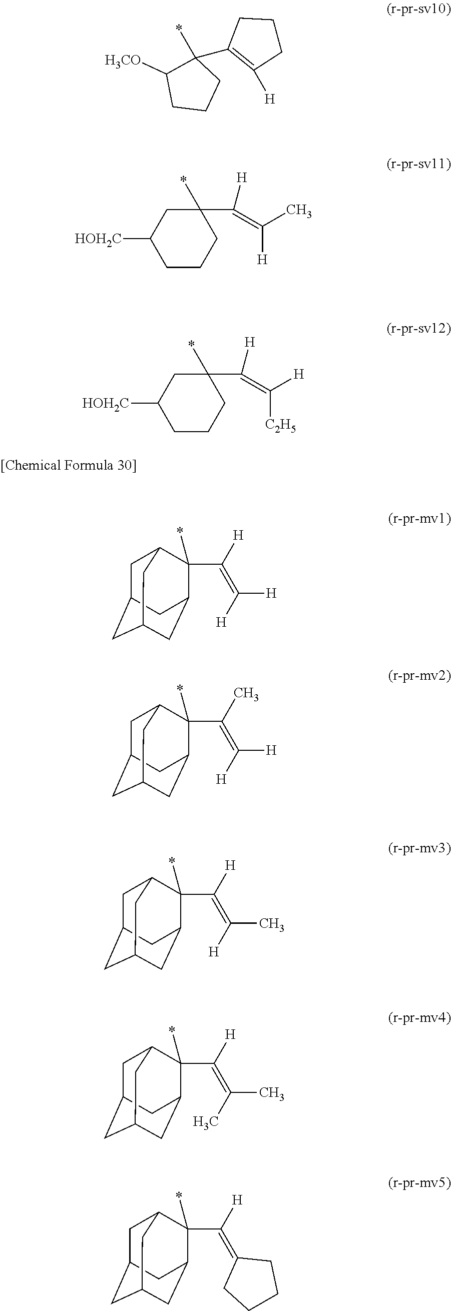

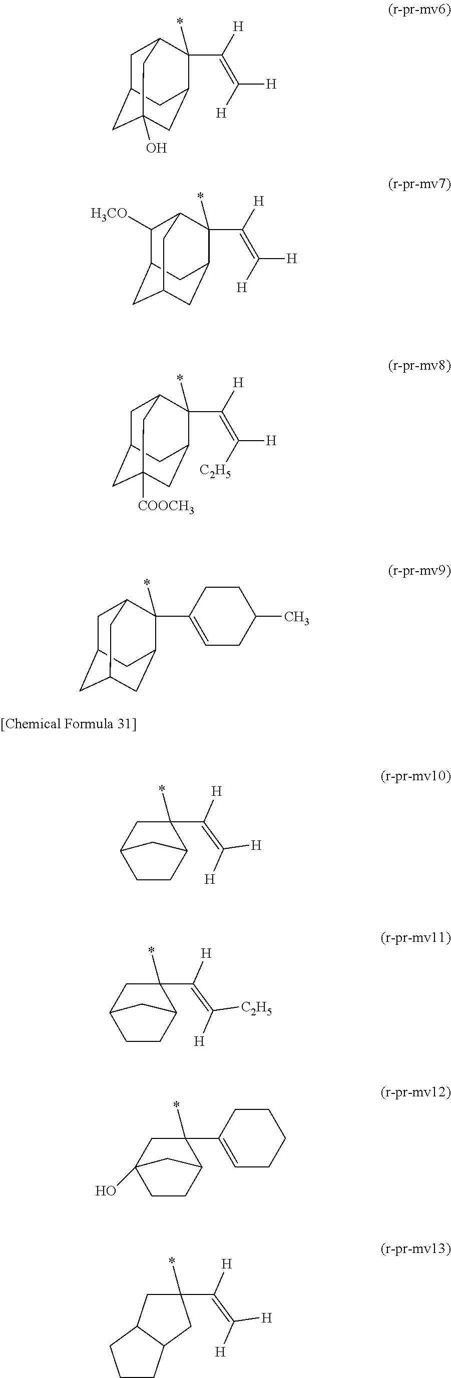

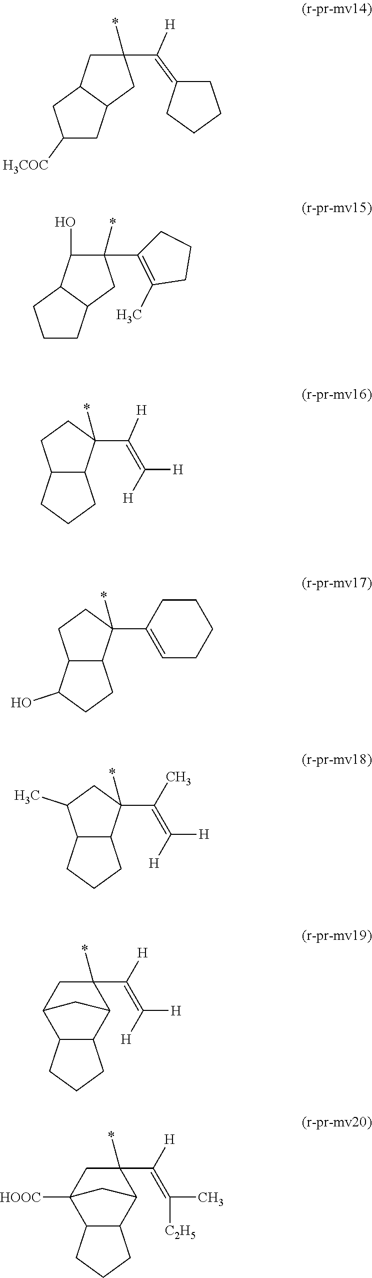

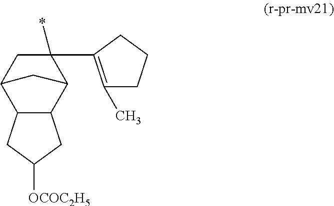

Specific examples of the group represented by Formula (a1-r2-3) are shown below.

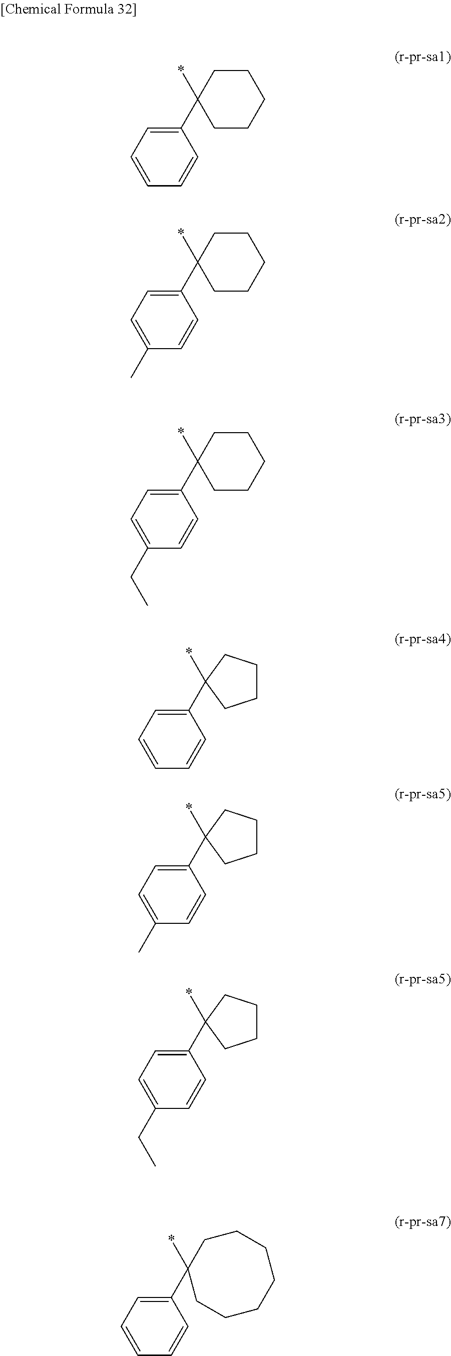

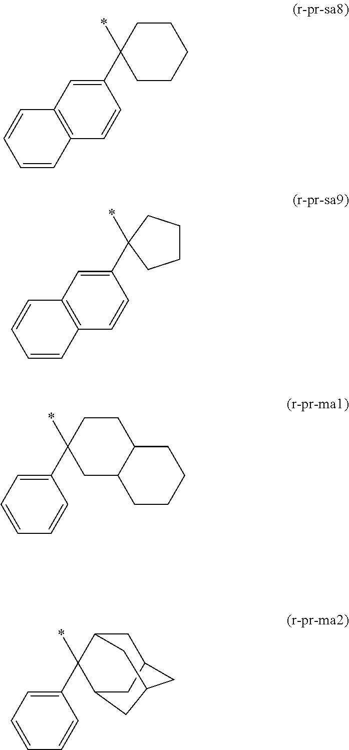 [SMILES: *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

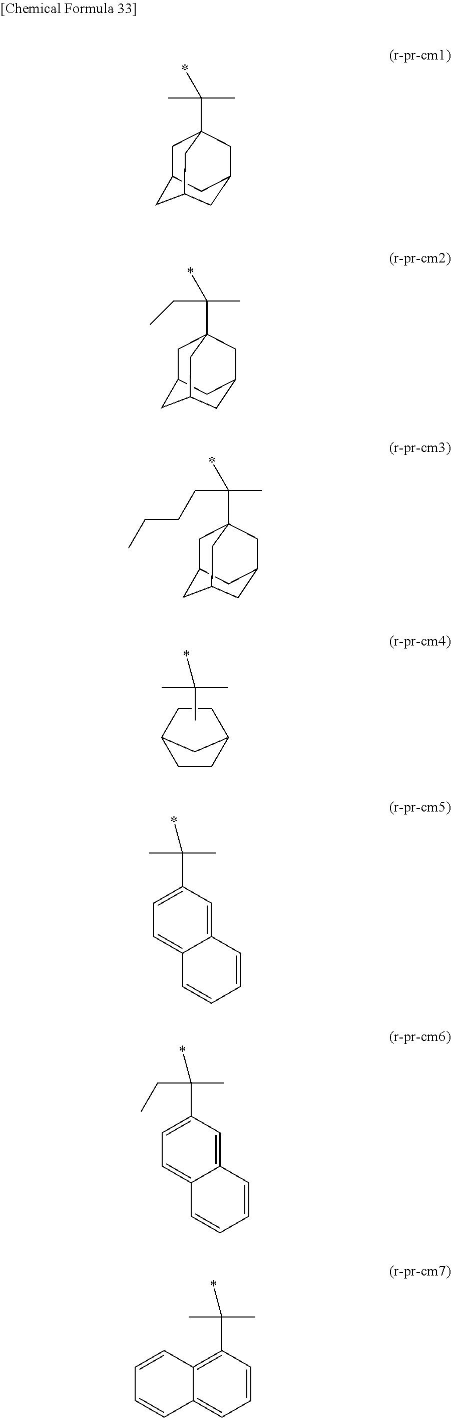

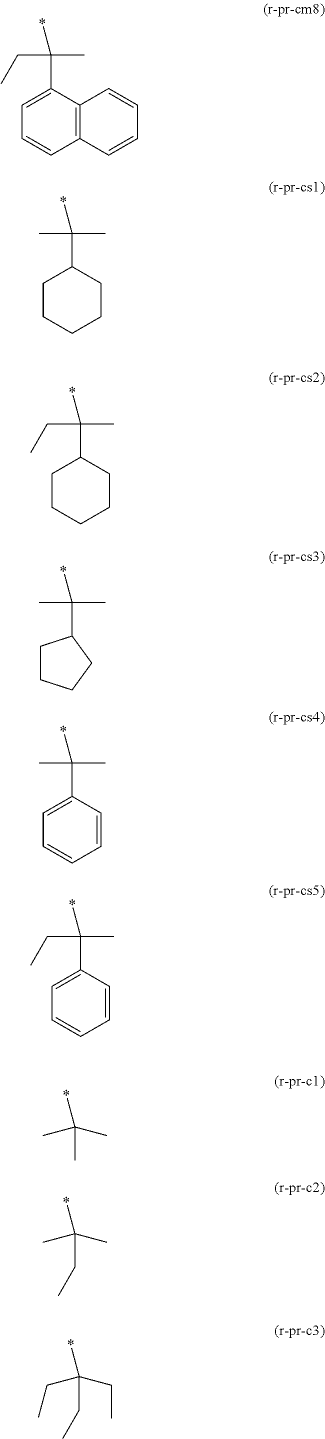

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

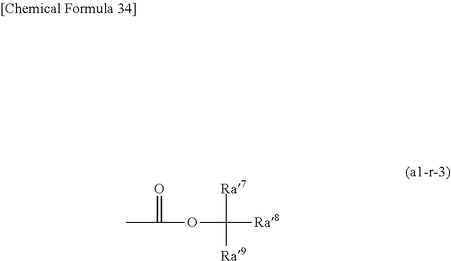

[In the formula, Ra′⁷ to Ra′⁹ each represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

In addition, the total number of carbon atoms in each of the alkyl groups is preferably in a range of 3 to 7, more preferably in a range of 3 to 5, and most preferably 3 or 4.

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least a part of hydrogen atoms in a hydroxyl group of a constitutional unit derived from hydroxystyrene or a hydroxystyrene derivative are protected by a substituent including the above-described acid decomposable group; and a constitutional unit in which at least a part of hydrogen atoms in —C(═O)—OH of a constitutional unit derived from vinylbenzoic acid or a vinylbenzoic acid derivative are protected by a substituent including the above-described acid decomposable group.

Among the above, the constitutional unit (a1) is preferably a constitutional unit derived from acrylic acid ester in which the hydrogen atom bonded to the carbon atom at the α-position may be substituted with a substituent.

Specific preferred examples of such constitutional unit (a1) include constitutional units represented by General Formula (a1-1) or (a1-2) shown below.

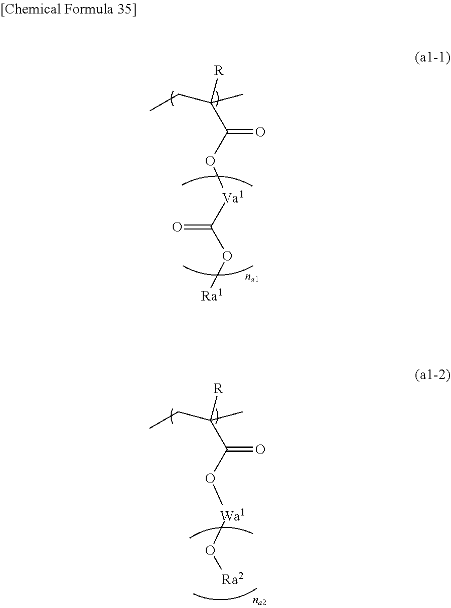

[In the formulae, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer in a range of 0 to 2. Ra¹ represents an acid dissociable group represented by General Formula (a1-r-1) or (a1-r-2). Wa¹ represents a (nₐ₂+1)-valent hydrocarbon group, nₐ₂ represents an integer in a range of 1 to 3, and Ra² represents an acid dissociable group represented by General Formula (a1-r-1) or (a1-r-3).]

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. The halogen atom is particularly preferably a fluorine atom.

R represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. Typically, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. The monocyclic alicyclic hydrocarbon group is preferably a group in which two hydrogen atoms have been removed from a monocycloalkane. The monocycloalkane has preferably 3 to 6 carbon atoms, and specific examples thereof include cyclopentane and cyclohexane. The polycyclic alicyclic hydrocarbon group is preferably a group obtained by removing two hydrogen atoms from a polycycloalkane, and the polycycloalkane is preferably a group having 7 to 12 carbon atoms. Specific examples of the polycycloalkane include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Provided that the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-4), Ra¹ represents an acid dissociable group represented by Formula (a1-r-1) or (a1-r-2).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. Typically, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent to tetravalent and more preferably divalent or trivalent.

In the Formula (a1-2), Ra² represents an acid dissociable group represented by General Formula (a1-r-1) or (a1-r-3).

Specific examples of the constitutional unit represented by Formula (a1-1) are shown below. In each of the formulae shown below, R^{α} represents a hydrogen atom, a methyl group, or a trifluoromethyl group.

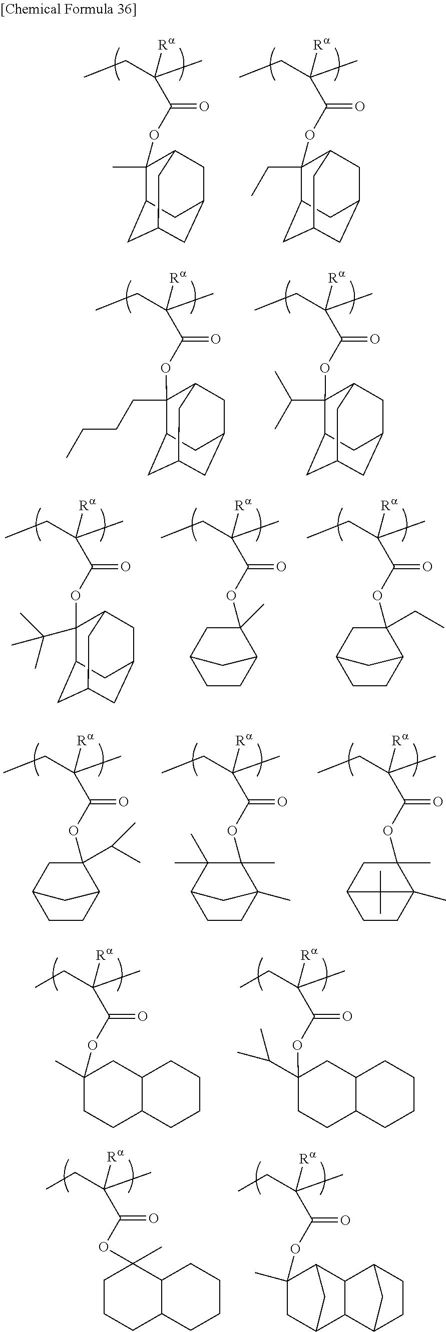

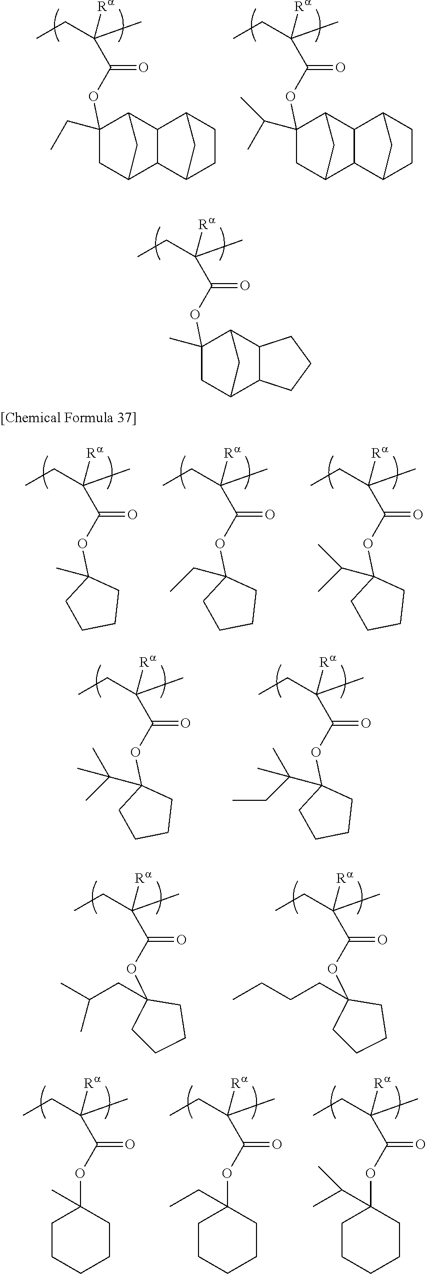

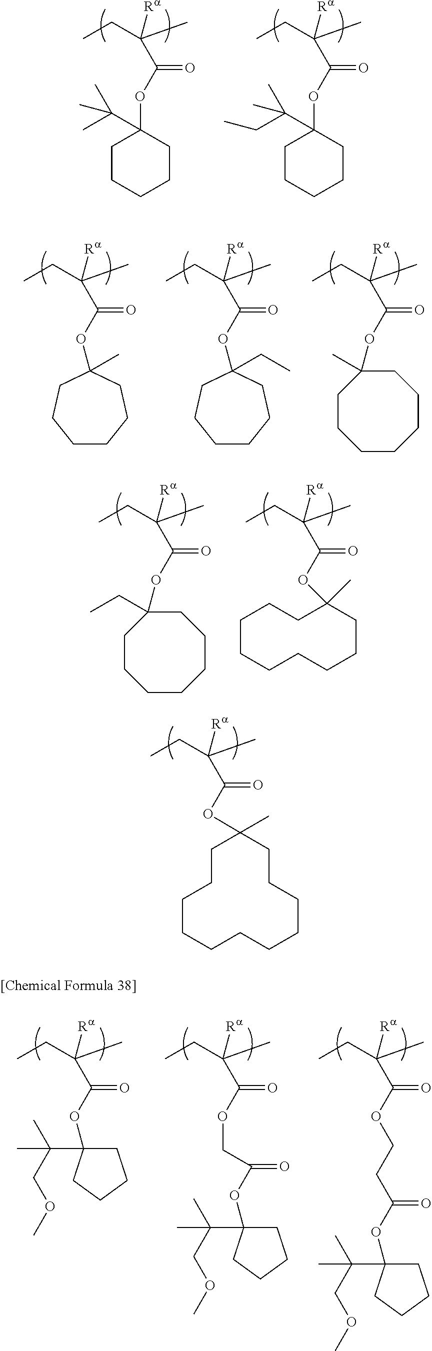

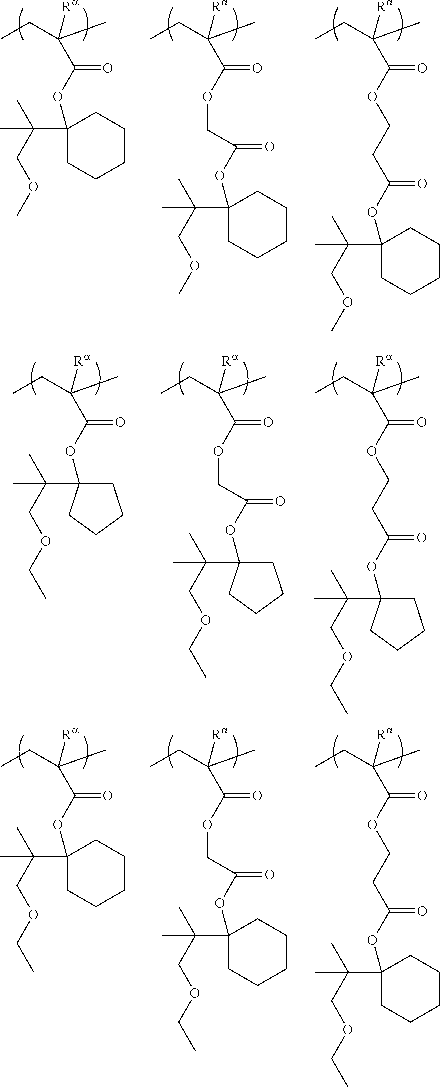

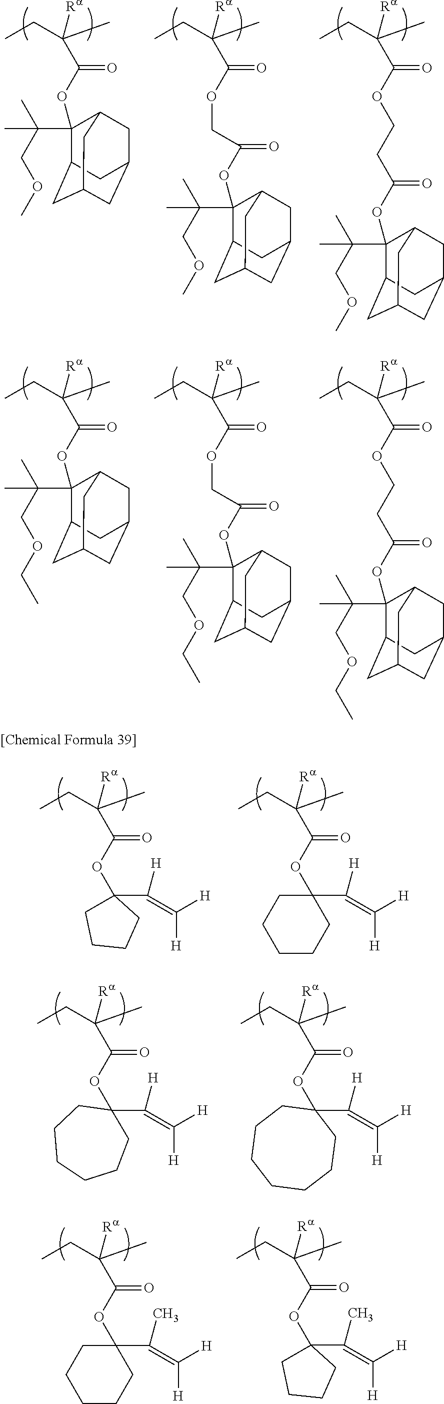

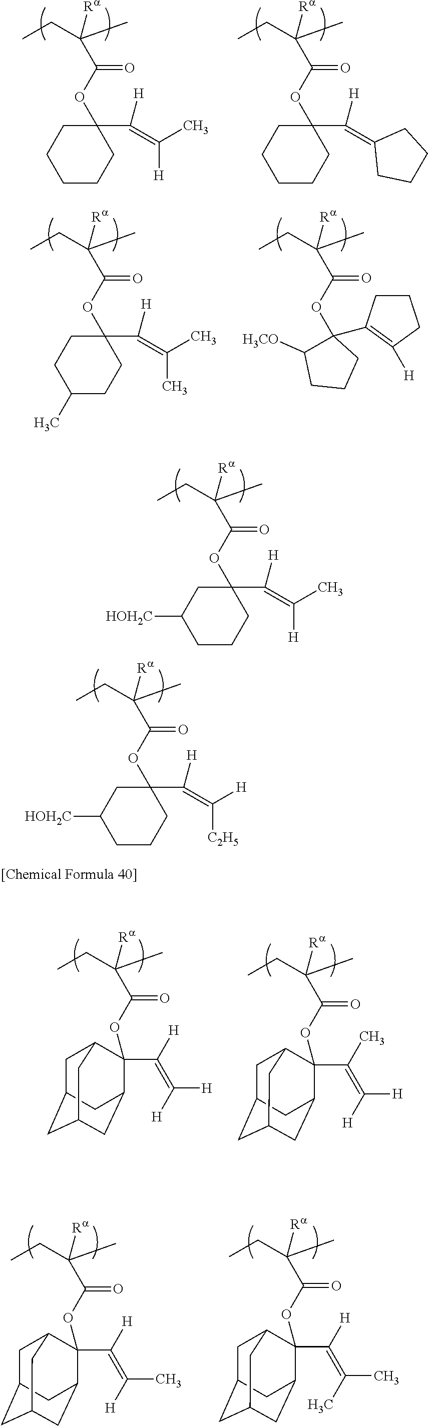

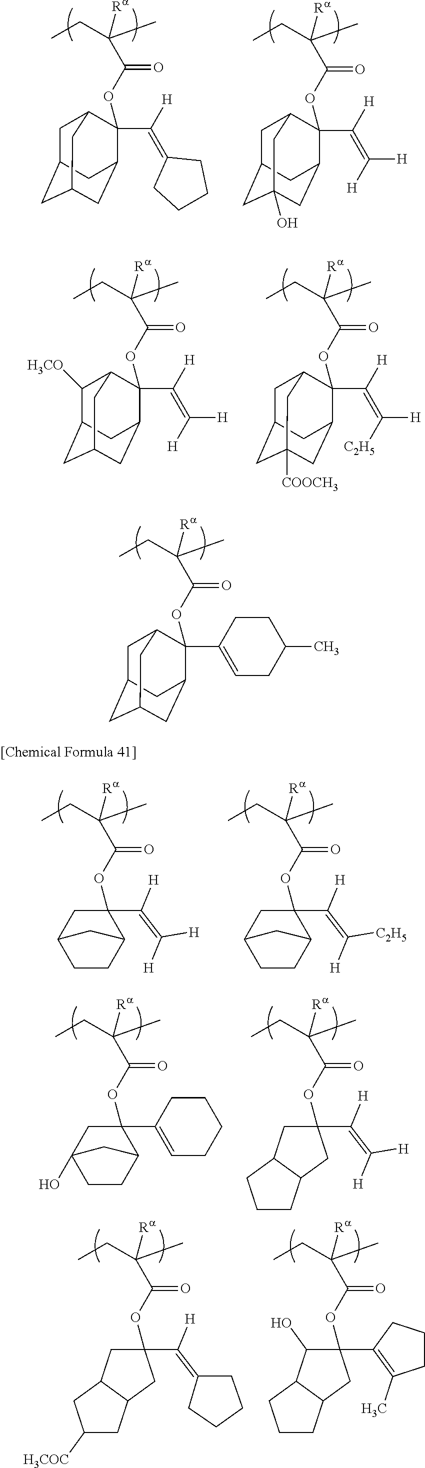

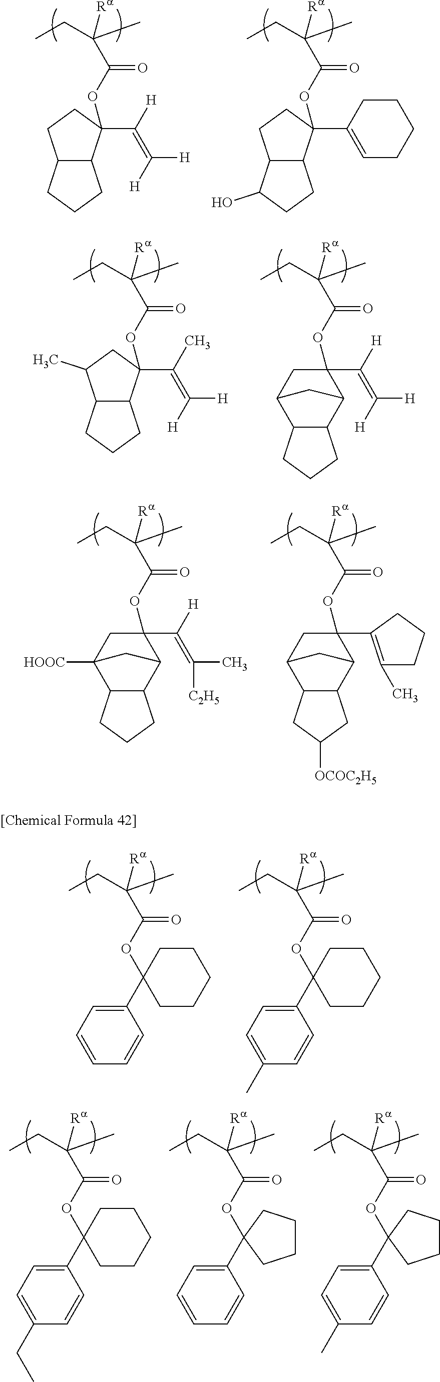

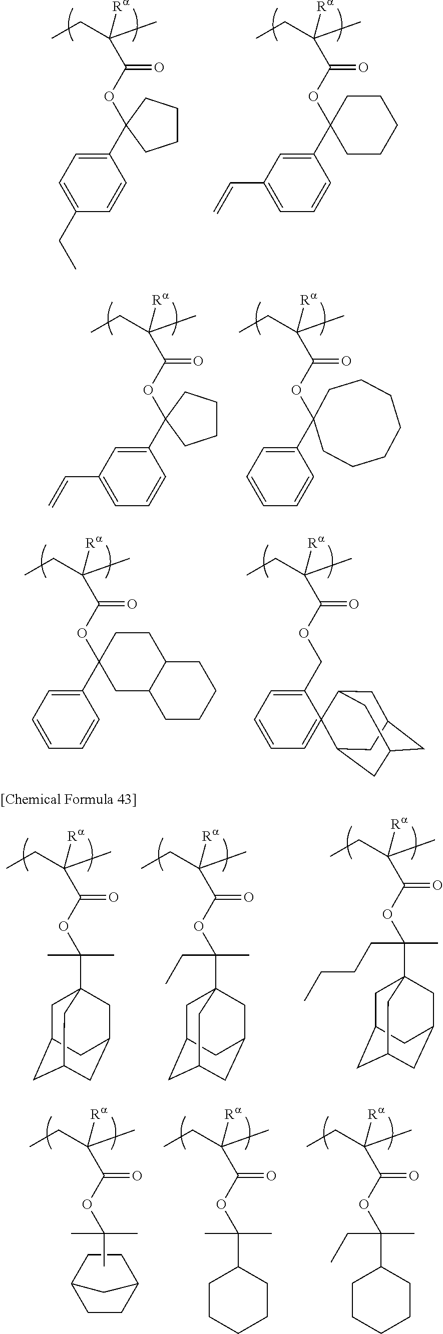

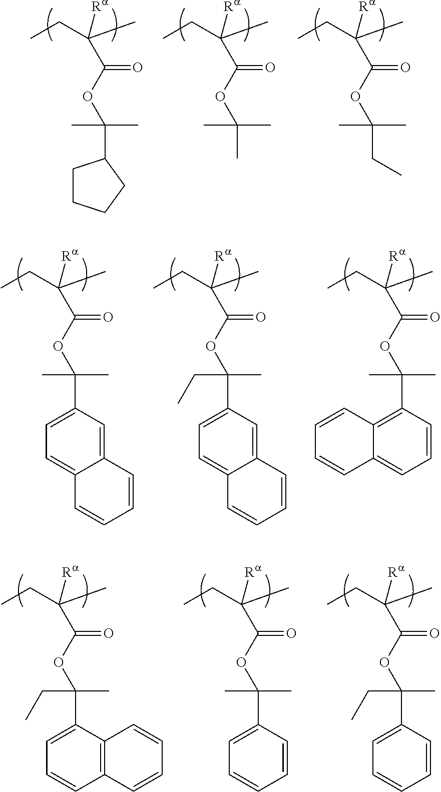

The constitutional unit (a1) included in the component (A1) may be used alone or two or more kinds thereof.

The constitutional unit (a1) is more preferably a constitutional unit represented by Formula (a1-1) since characteristics (sensitivity, shape, and the like) in lithography with an electron beam or EUV can be more easily enhanced.

Among these, the constitutional unit (a1) particularly preferably includes a constitutional unit represented by General Formula (a1-1-1) shown below.

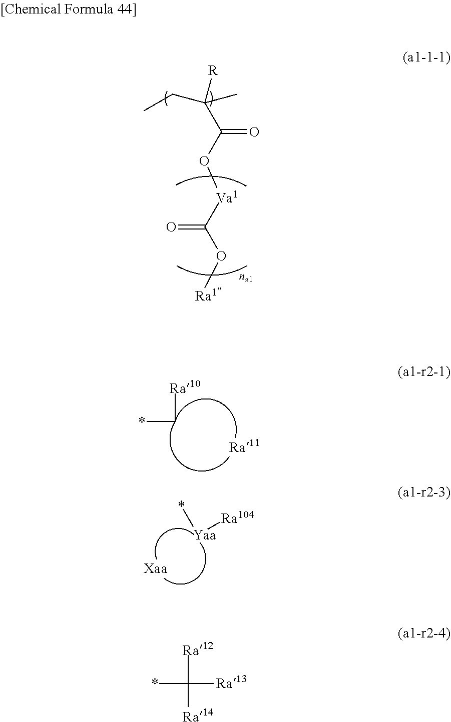

[In the formulae, Ra¹″ represents an acid dissociable group represented by General Formula (a1-r2-1), (a1-r2-3), or (a1-r2-4).]

R, Va¹, and nₐ₁ in Formula (a1-1-1) each have the same definition as that for R, Va¹, and nₐ₁ in Formula (a1-1).

The description of the acid dissociable group represented by General Formula (a1-r2-1), (a1-r2-3), or (a1-r2-4) is the same as described above. Among these, it is preferable to select those in which the acid dissociable group is a cyclic group since the reactivity is enhanced for EB or EUV, which is suitable.

In Formula (a1-1-1), it is preferable that Ra¹″ represents an acid dissociable group represented by General Formula (a1-r2-1) among the examples described above.

The proportion of the constitutional unit (a1) in the component (A1) is preferably in a range of 5% to 99% by mole, more preferably in a range of 10% to 95% by mole, still more preferably in a range of 30% to 90% by mole, and particularly preferably in a range of 40% to 85% by mole, with respect to the total amount (100% by mole) of all constitutional units constituting the component (A1).

In a case where the proportion of the constitutional unit (a1) is set to be equal to or more than the lower limit values of the above-described preferable ranges, lithography characteristics such as the sensitivity, CDU, the resolution, and reduction of the roughness are improved. On the other hand, in a case where the proportion is equal to or less than the upper limit values of the above-described preferred ranges, balance with other constitutional units can be obtained, and various lithography characteristics are improved.

### <<Other Constitutional Units>>

The component (A1) may have other constitutional units as necessary in addition to the constitutional unit (a0) and the constitutional unit (a1), which are described above.

Examples of the other constitutional units include a constitutional unit (a10) represented by General Formula (a10-1) described later; a constitutional unit (a2) containing a lactone-containing cyclic group; and a constitutional unit (a8) derived from a compound represented by General Formula (a8-1) described later.

In Regard to Constitutional Unit (a10):

The constitutional unit (a10) is a constitutional unit represented by General Formula (a10-1).

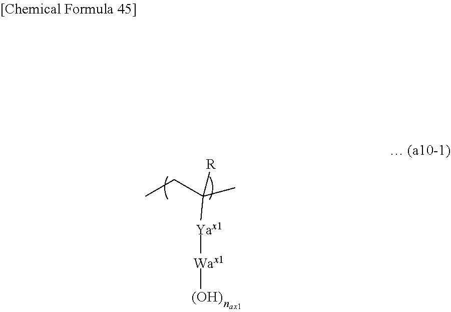

[In the formulae, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or more.]

In the Formula (a10-1), R represents a hydrogen atom, an alkyl group having 1 to 5 carbon atoms, or a halogenated alkyl group having 1 to 5 carbon atoms.

R represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a hetero atom.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent. The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

In addition, examples of the aromatic hydrocarbon group as Wa^{x1} also include a group in which (nₐₓ₁+1) hydrogen atoms have been removed from an aromatic compound having an aromatic ring (for example, biphenyl or fluorene) which may have two or more substituents.

Among the above, Wa^{x1} represents preferably a group obtained by removing (nₐₓ₁+1) hydrogen atoms from benzene, naphthalene, anthracene, or biphenyl, more preferably a group obtained by removing (nₐₓ₁+1) hydrogen atoms from benzene or naphthalene, and still more preferably a group obtained by removing (nₐₓ₁+1) hydrogen atoms from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include those exemplified as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or more, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In each of the formulae shown below, R^{α} represents a hydrogen atom, a methyl group, or a trifluoromethyl group.

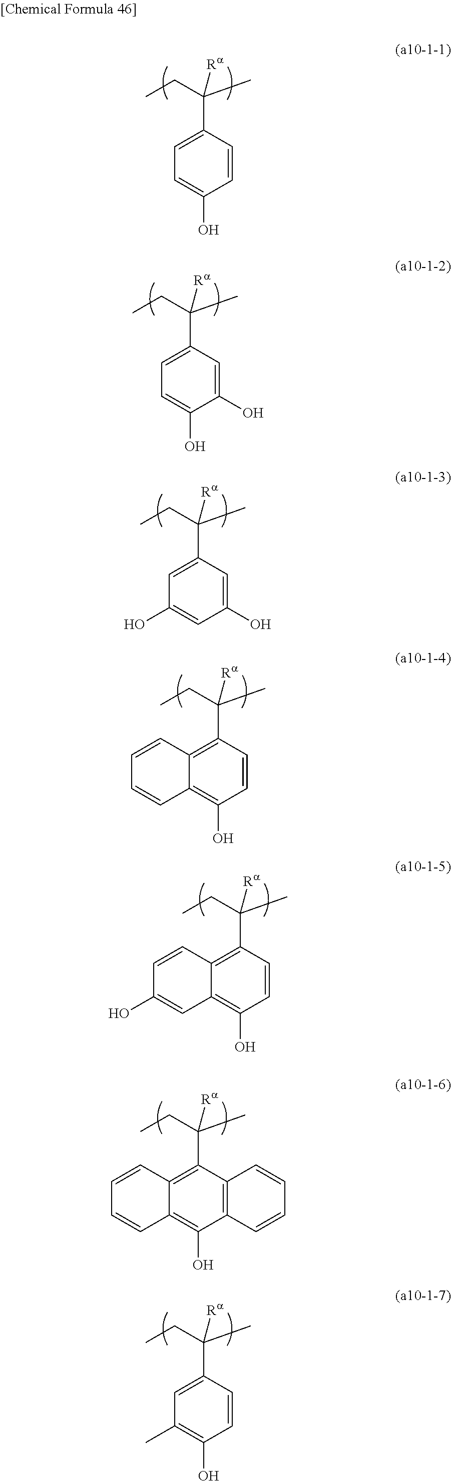

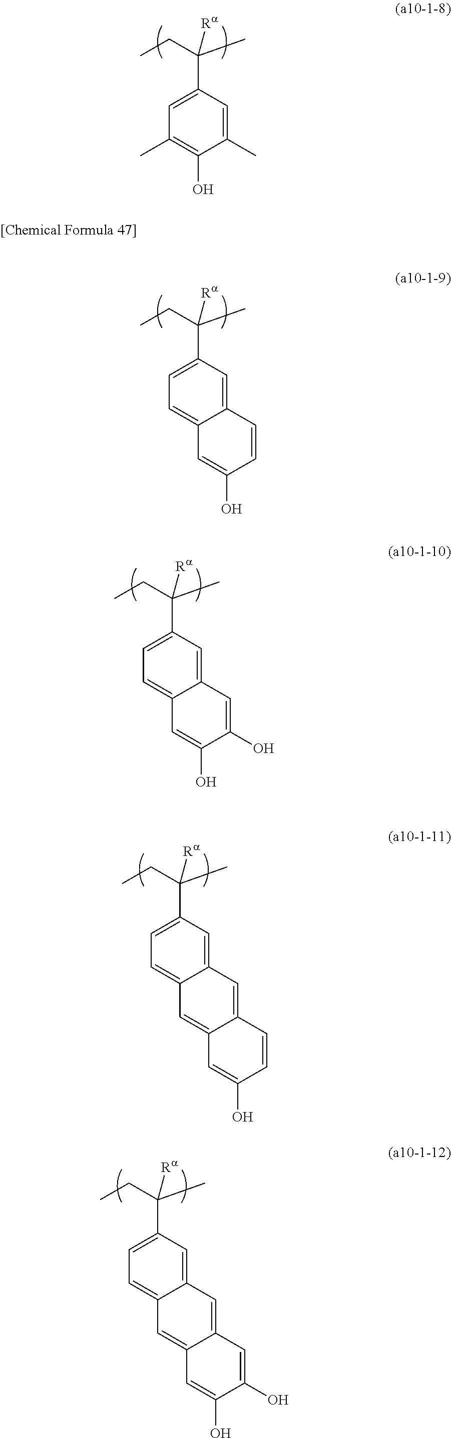

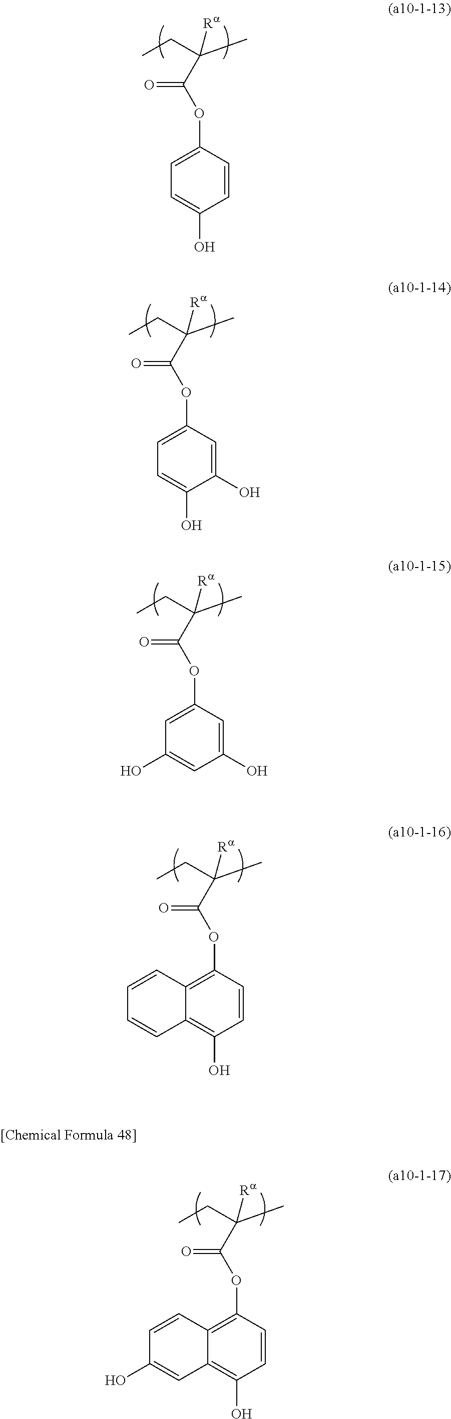

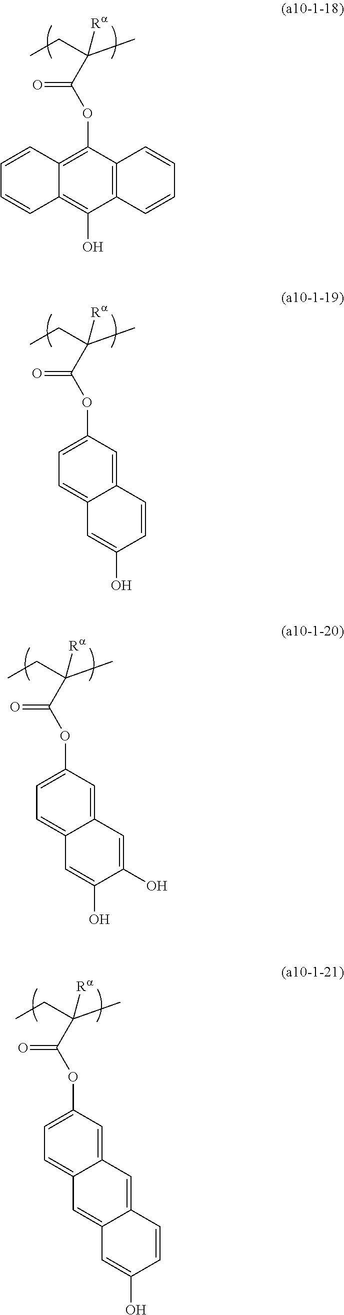

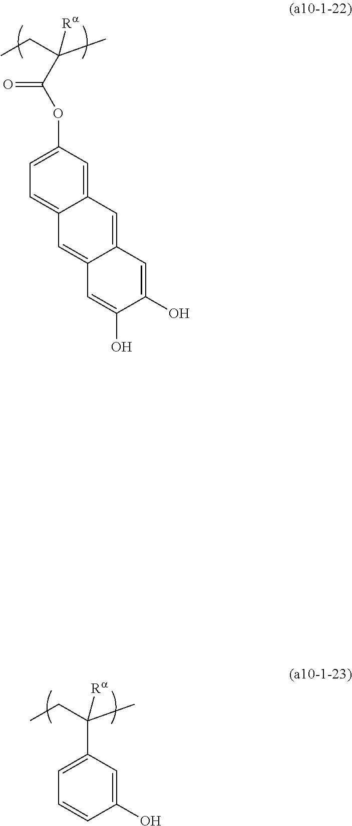

The constitutional unit (a10) included in the component (A1) may be used alone or two or more kinds thereof.

In a case where the component (A1) has the constitutional unit (a110), the proportion of the constitutional unit (a10) in the component (A1) is preferably in a range of 5% to 95% by mole, more preferably in a range of 110% to 90% by mole, still more preferably in a range of 10% to 70% by mole, and particularly preferably in a range of 10% to 60% by mole, with respect to the total amount (100% by mole) of all constitutional units constituting the component (A1).

In a case where the proportion of the constitutional unit (a10) is set to be equal to or more than the lower limit value, the sensitivity is easily to be enhanced. On the other hand, in a case where the proportion thereof is equal to or less than the upper limit value, balance with other constitutional units is obtained easily.

### In Regard to Constitutional Unit (a2):

The component (A1) may further have a constitutional unit (a2) (here, a constitutional unit corresponding to the constitutional unit (a1) is excluded) containing a lactone-containing cyclic group, in addition to the constitutional unit (a0) and the constitutional unit (a1).

In a case where the component (A1) is used to form a resist film, the lactone-containing cyclic group of the constitutional unit (a2) is effective for enhancing the adhesiveness of the resist film to the substrate. In addition, in a case of having the constitutional unit (a2), lithography characteristics can be improved, for example, by the effects obtained by appropriately adjusting the acid diffusion length, enhancing the adhesiveness of the resist film to the substrate, and appropriately adjusting the solubility during development.

The term “lactone-containing cyclic group” indicates a cyclic group that has a ring (lactone ring) containing —O—C(═O)— in the ring skeleton. The lactone ring is counted as the first ring, and in a case where the group contains only the lactone ring, the group is referred to as a monocyclic group, or in a case where the group further has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

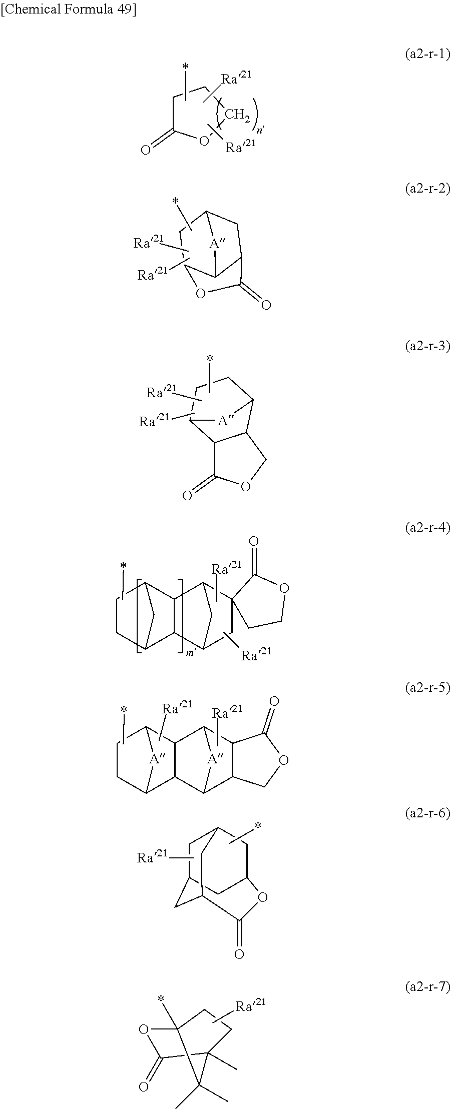

[In the formulae, Ra′²¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer in a range of 0 to 2, and m′ is 0 or 1.]

In General Formulae (a2-r-1) to (a2-r-7), it is preferable that the alkyl group as Ra′²¹ is an alkyl group having 1 to 6 carbon atoms. The alkyl group is preferably a linear alkyl group or a branched alkyl group. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. It is preferable that the alkoxy group is linear or branched. Specific examples of the alkoxy groups include a group formed by linking the above-described alkyl group exemplified as the alkyl group represented by Ra′²¹ to an oxygen atom (—O—).

The halogen atom as Ra′²¹ is preferably a fluorine atom.

Examples of the halogenated alkyl group as Ra′²¹ include groups in which some or all hydrogen atoms in the alkyl group as Ra′²¹ have been substituted with the halogen atoms. The halogenated alkyl group is preferably a fluorinated alkyl group and particularly preferably a perfluoroalkyl group.

In —COOR″ and —OC(═O)R″ as Ra′²¹, each R″ represents a hydrogen atom, an alkyl group, or a lactone-containing cyclic group at the same time.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the cyclic alkyl group preferably has 3 to 15 carbon atoms, more preferably has 4 to 12 carbon atoms, and most preferably has 5 to 10 carbon atoms. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

The hydroxyalkyl group as Ra′²1 preferably has 1 to 6 carbon atoms, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

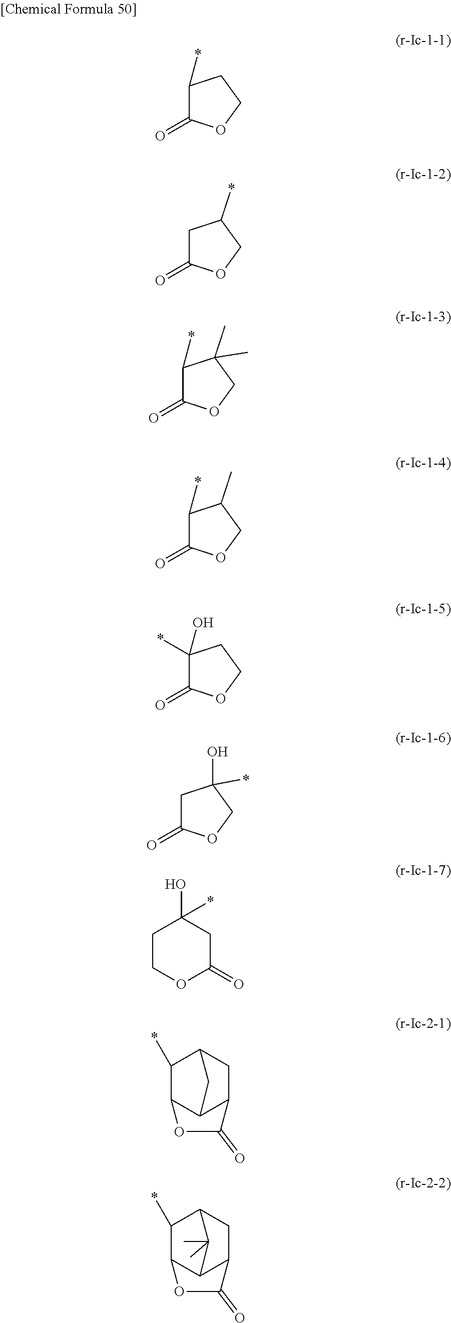

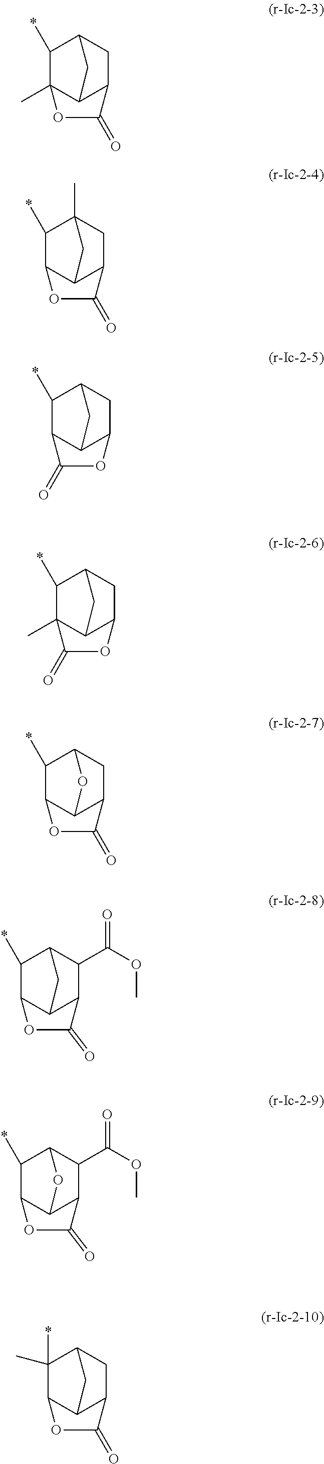

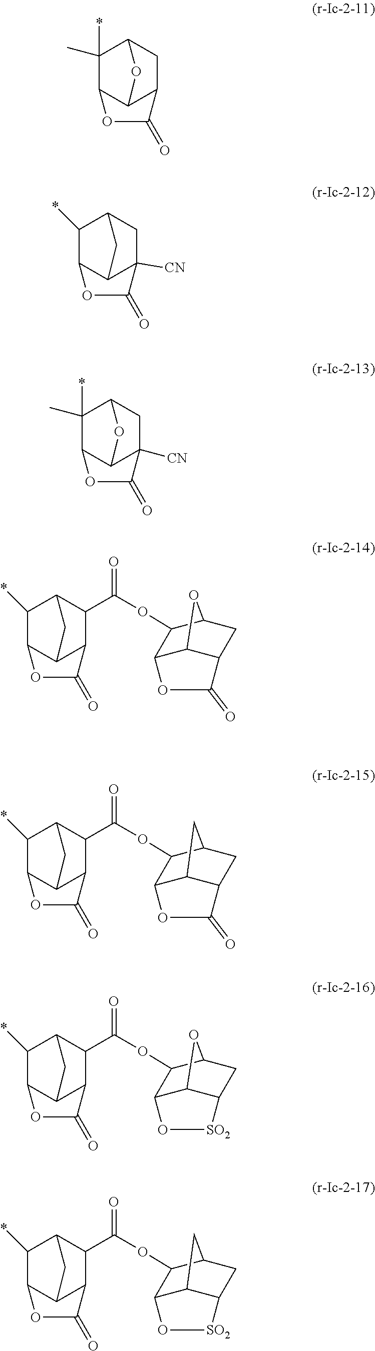

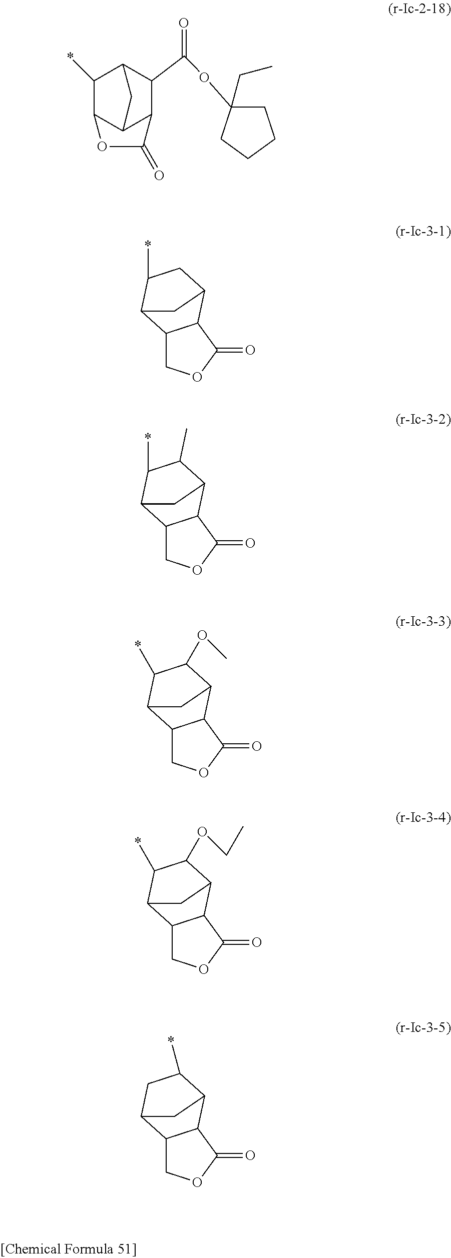

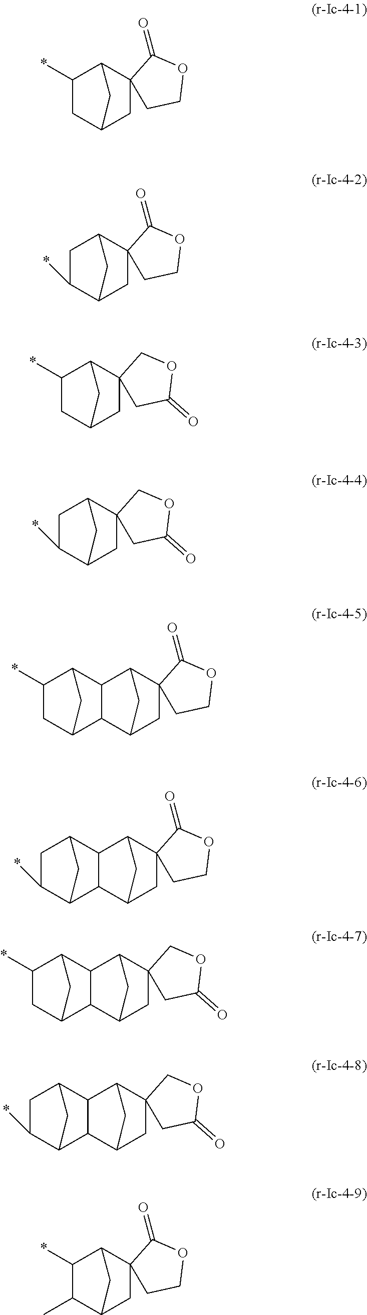

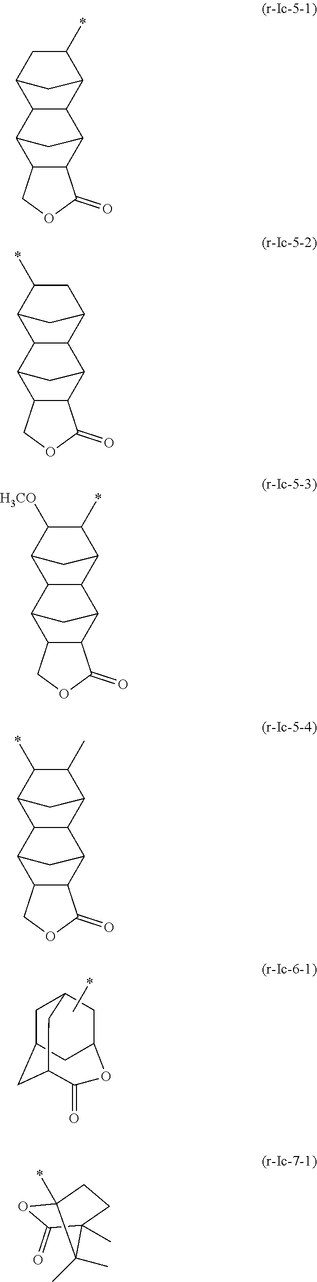

As the constitutional unit (a2), a constitutional unit derived from acrylic acid ester in which the hydrogen atom bonded to the carbon atom at the α-position may be substituted with a substituent is preferable.

It is preferable that such constitutional unit (a2) is a constitutional unit represented by General Formula (a2-1).

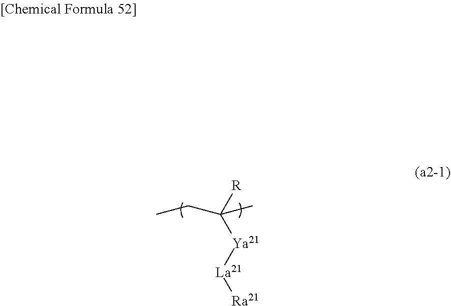

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²′ represents —O—, —COO—, —CON(R′)—, —OCO—, —CONHCO—, or —CONHCS—, and R′ represents a hydrogen atom or a methyl group. In a case where La²′ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a hetero atom.

It is preferable that Ya²¹ represents a single bond, an ester bond [—C(═O)—O—], an ether bond (—O—), a linear or branched alkylene group, or a combination thereof.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

Among these, groups each represented by General Formulae (a2-r-1), (A2-r-2), and (a2-r-6) are preferable, and a group represented by General Formula (a2-r-2) is more preferable. Specifically, any one of groups each represented by Chemical Formulae (r-Ic-1-1) to (r-lc-1-7), (r-lc-2-1) to (r-lc-2-18), and (r-lc-6-1) is preferable, any one of groups each represented by Chemical Formulae (r-ic-2-1) to (r-lc-2-18) is more preferable, and any one of groups each represented by Chemical Formulae (r-lc-2-1) and (r-lc-2-12) is still more preferable.

The constitutional unit (a2) included in the component (A1) may be used alone or two or more kinds thereof.

In a case where the component (A1) has the constitutional unit (a2), the proportion of the constitutional unit (a2) is preferably in a range of 5% to 60% by mole, more preferably in a range of 10% to 60% by mole, still more preferably in a range of 20% to 60% by mole, and particularly preferably in a range of 30% to 60% by mole with respect to the total amount (100% by mole) of all constitutional units constituting the component (A1).

In a case where the proportion of the constitutional unit (a2) is set to be equal to or more than the lower limit values of the above-described preferable ranges, the effect to be obtained by allowing the component (A1) to have the constitutional unit (a2) is sufficiently obtained by the above-described effects. Further, in a case where the proportion thereof is set to be equal to or less than the upper limit values of the above-described preferable ranges, the constitutional unit (a2) and other constitutional units can be balanced, and the lithography characteristics are improved.

### In Regard to Constitutional Unit (a8):

The constitutional unit (a8) is a constitutional unit derived from a compound represented by General Formula (a8-1).

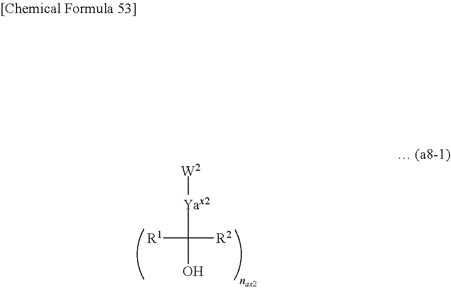

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms which may have a fluorine atom or a hydrogen atom. R² and Ya¹² may be bonded to each other to form a ring structure. nₐₓ₂ represents an integer in a range of 1 to 3.]

The term “polymerizable group” in the polymerizable group-containing group as W² denotes a group that enables a compound containing a polymerizable group to be polymerized by radical polymerization or the like, which is, for example, a group having a multiple bond between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group containing a hetero atom.

Suitable examples of the polymerizable group-containing group include a group represented by Chemical Formula: C(R^{X11})(R^{X12})═C(R^{X13})—Ya^{x0}.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya² and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the formulae described below, R^{α} represents a hydrogen atom, a methyl group, or a trifluoromethyl group.

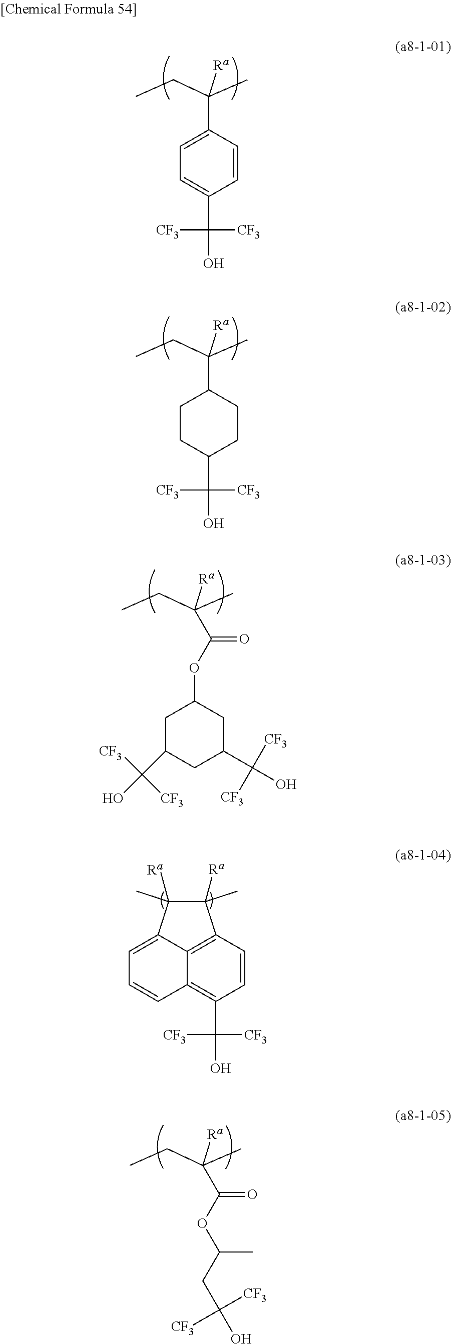

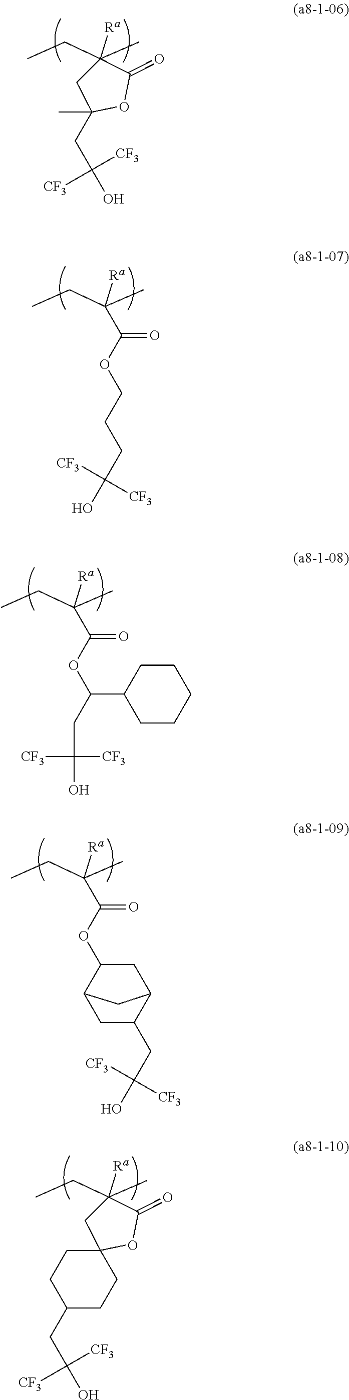

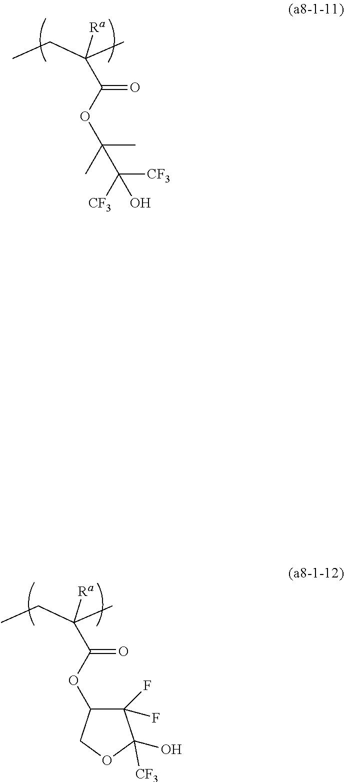

Among the above examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10), and more preferably at least one selected from the group consisting of constitutional units each represented by Chemical Formulae (a8-1-01) to (a8-1-04) and (a8-1-09).

The constitutional unit (a8) contained in the component (A1) may be used alone or two or more kinds thereof.

In a case where the component (A1) has the constitutional unit (a8), the proportion of the constitutional unit (a8) is preferably in a range of 1% to 50% by mole, more preferably in a range of 5% to 45% by mole, and still more preferably in a range of 5% to 40% by mole, with respect to the total (100% by mole) of all constitutional units constituting the component (A1).

In a case where the proportion of the constitutional unit (a8) is equal to or more than the preferred lower limit value, the compatibility with the developing solution and the rinse liquid can be enhanced. On the other hand, in a case where the proportion is equal to or less than the upper limit value of the preferred ranges, balance with other constitutional units can be obtained, and various lithography characteristics are improved.

The component (A1) contained in the resist composition may be used alone or in combination of two or more kinds thereof.

In the resist composition according to the present embodiment, examples of the component (A1) include a polymer compound having a repeating structure of the constitutional unit (a0) and the constitutional unit (a1), and preferred examples of the component (A1) include a polymer compound having a repeating structure of the constitutional unit (a0), the constitutional unit (a1), and the constitutional unit (a10).

Among the above, suitable examples of the component (A1) include a polymer compound consisting of a repeating structure of the constitutional unit (a0) and the constitutional unit (a1) and a polymer compound consisting of a repeating structure of the constitutional unit (a0), the constitutional unit (a1), and the constitutional unit (a10).

In the polymer compound having a repeating structure of the constitutional unit (a0) and the constitutional unit (a1), the proportion of the constitutional unit (a0) is preferably in a range of 1% to 50% by mole, more preferably in a range of 5% to 40% by mole, still more preferably in a range of 10% to 30% by mole, and particularly preferably in a range of 15% to 25% by mole with respect to the total amount (100% by mole) of all constitutional units constituting the polymer compound.

In addition, the proportion of the constitutional unit (a1) in the polymer compound is preferably in a range of 50% to 99% by mole, more preferably in a range of 60% to 95% by mole, still more preferably in a range of 70% to 90% by mole, and particularly preferably in a range of 75% to 85% by mole, with respect to the total amount (100% by mole) of all constitutional units constituting the polymer compound.

In the polymer compound having a repeating structure of the constitutional unit (a), the constitutional unit (a1), and the constitutional unit (a10), the proportion of the constitutional unit (a0) is preferably in a range of 1% to 35% by mole, more preferably in a range of 3% to 30% by mole, still more preferably in a range of 5% to 25% by mole, and particularly preferably in a range of 10% to 20% by mole, with respect to the total amount (100% by mole) of all constitutional units constituting the polymer compound.

In addition, the proportion of the constitutional unit (a1) in the polymer compounds is preferably in a range of 45% to 90% by mole, more preferably in a range of 50% to 85% by mole, still more preferably in a range of 55% to 80% by mole, and particularly preferably in a range of 60% to 75% by mole, with respect to the total amount (100% by mole) of all constitutional units constituting the polymer compound.

In addition, the proportion of the constitutional unit (a10) in the polymer compound is preferably in a range of 5% to 35% by mole, more preferably in a range of 5% to 30% by mole, still more preferably in a range of 5% to 25% by mole, and particularly preferably in a range of 10% to 20% by mole, with respect to the total amount (100% by mole) of all constitutional units constituting the polymer compound.

Such component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, such component (A1) can be produced by dissolving, in a polymerization solvent, a monomer from which the constitutional unit (a1) is derived and, as necessary, a monomer from which a constitutional unit (for example, the constitutional unit (a10)) other than the constitutional unit (a1) is derived, adding thereto a radical polymerization initiator as described above to carry out polymerization, and then carrying out a deprotection reaction.

A —C(CF₃)₂—OH group may be introduced into the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1,000 to 50,000, more preferably in a range of 2,000 to 30,000, and still more preferably in a range of 3,000 to 20,000.

In a case where the Mw of the component (A1) is equal to or less than the upper limit value of the preferred range, a sufficient solubility in a resist solvent to be used as a resist is exhibited. On the contrary, in a case where the Mw of the component (A1) is equal to or more than the lower limit value of the preferred range, the dry etching resistance and the cross-sectional shape of the resist pattern are satisfactory.

The dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Mn represents the number-average molecular weight.

### In Regard to Component (A2)

In the resist composition according to the present embodiment, a base material component (hereinafter, referred to as a “component (A2)”) whose solubility in a developing solution is changed due to the action of an acid, which does not correspond to the component (A1), may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or more, more preferably 50% by mass or more, and still more preferably 75% by mass or more, and may be 100% by mass with respect to the total mass of the component (A). In a case where the proportion thereof is 25% by mass or more, a resist pattern having excellent various lithography characteristics such as increased sensitivity, resolution, and improved roughness is likely to be formed.

In the resist composition according to the present embodiment, the content of the component (A) may be adjusted according to the thickness of the resist film intended to be formed.

### <Acid Generator Component (B)>

The resist composition according to the present embodiment further contains an acid generator component (B) that generates acid upon exposure to light in addition to the component (A).

The component (B) is not particularly limited, and those which have been proposed so far as an acid generator for a chemically amplified resist composition in the related art can be used.

Examples of such a acid generator include numerous acid generators such as onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, iminosulfonate-based acid generators, and disulfonate-based acid generators.

Examples of the onium salt-based acid generators include a compound represented by General Formula (b-1) (hereinafter, also referred to as “component (b-1”), a compound represented by General Formula (b-2) (hereinafter, also referred to as “component (b-2”), and a compound represented by General Formula (b-3) (hereinafter, also referred to as “component (b-3”).

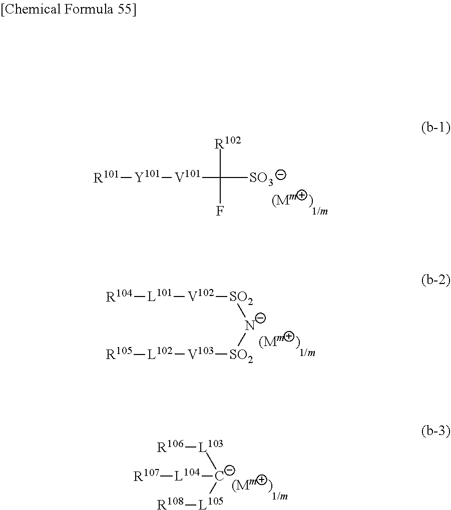

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵, may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰³ to L¹⁰⁵ each independently represent a single bond, —CO—, or —SO₂—. m represents an integer of 1 or more, and M^{m+} represents an m-valent onium cation.]

### {Anion Moiety}

Anion in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group with no aromaticity. In addition, the aliphatic hydrocarbon group may be saturated or unsaturated. Typically, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Provided that the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane has preferably 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms. Among these, a polycycloalkane having a crosslinked ring polycyclic skeleton such as adamantane, norbornane, isobornane, tricyclodecane, or tetracyclododecane; and a polycycloalkane having a condensed ring-based polycyclic skeleton such as a cyclic group having a steroid skeleton are preferable as the polycycloalkane.

Among these, the cyclic aliphatic hydrocarbon group as R^{1′}0 is preferably a group obtained by removing one or more hydrogen atoms from a monocycloalkane or a polycycloalkane, more preferably a group obtained by removing one hydrogen atom from a polycycloalkane, still more preferably adamantly group or a norbornyl group, and particularly preferably adamantly group.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, the cyclic hydrocarbon group as R¹⁰¹ may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and other heterocyclic groups each represented by Chemical Formulae (r-hr-1) to (r-hr-16). In the formulae, * represents a bonding site bonded to Y¹⁰¹ in Formula (b-1).

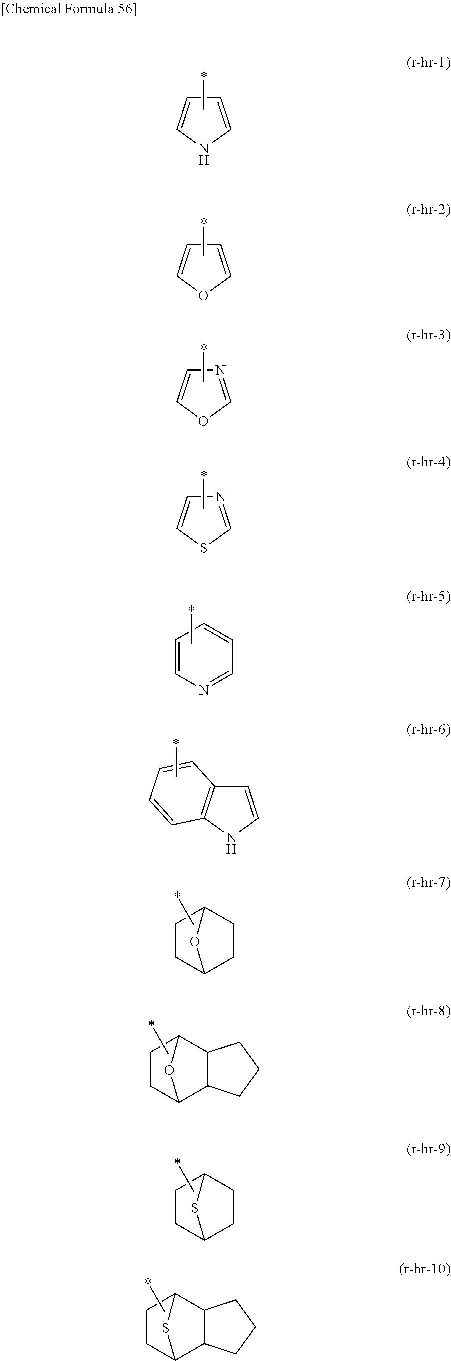

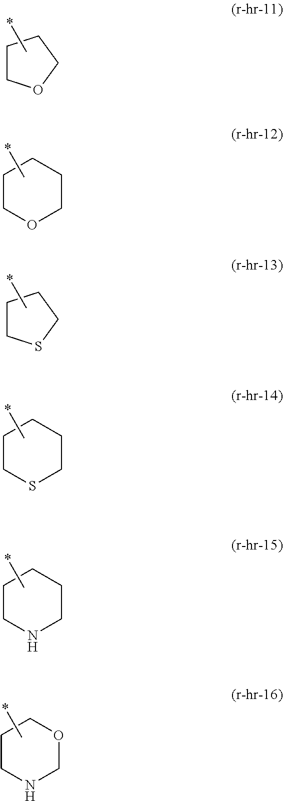

Examples of the substituent for the cyclic group as R¹⁰¹ include an alkyl group, an alkoxy group, a halogen atom, a halogenated alkyl group, a hydroxyl group, a carbonyl group, and a nitro group.

The alkyl group as the substituent is preferably an alkyl group having 1 to 5 carbon atoms, and a methyl group, an ethyl group, a propyl group, an n-butyl group, or a tert-butyl group is most preferable.

The alkoxy group as the substituent preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom as the substituent include a fluorine atom, a chlorine atom, a bromine atom, and an iodine atom. Among these, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group having a condensed ring in which an aliphatic hydrocarbon ring and an aromatic ring are condensed. Examples of the condensed ring include those obtained by condensing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. The condensed ring type is preferably a group containing a condensed ring, in which two or three aromatic rings are condensed with a bicycloalkane, and more preferably a group containing a condensed ring in which two or three aromatic rings are condensed with bicyclo[2.2.2]octane. Specific examples of the condensed cyclic group as R¹⁰¹ include those represented by Formulae (r-br-1) and (r-br-2). In the formulae, * represents a bonding site bonded to Y¹⁰¹ in Formula (b-1).

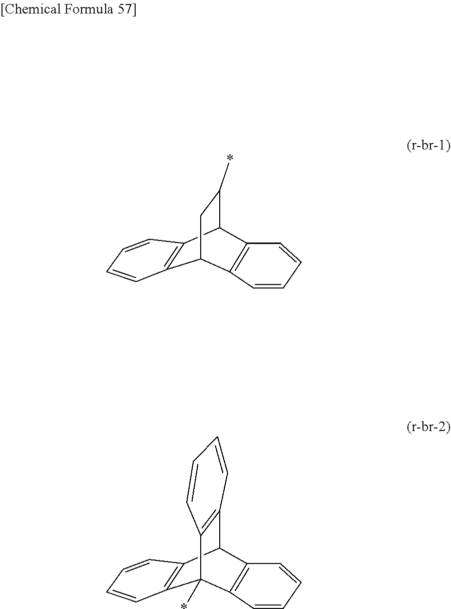 [SMILES: *C1CC2c3ccccc3C1c1ccccc12 ; *C12c3ccccc3C(c3ccccc31)c1ccccc12]

Examples of the substituent which may be contained in the condensed cyclic group as R¹⁰¹ include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those exemplified as the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane; a group in which one hydrogen atom has been removed from a polycycloalkane such as adamantane, norbornane, isobornane, tricyclodecane, or tetracyclododecane; a lactone-containing cyclic group represented by any of General Formulae (a2-r-1) to (a2-r-7); a —SO₂-containing cyclic group represented by any of General Formulae (a5-r-1) to (a5-r-4); and a heterocyclic group represented by any of Formulae (r-hr-7) to (r-hr-16).

Chain-Like Alkyl Group which May have Substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butenyl group. Examples of the branched alkenyl group include a 1-methylvinyl group, a 2-methylvinyl group, a 1-methylpropenyl group, and a 2-methylpropenyl group.

Among the above, the chain-like alkenyl group is preferably a linear alkenyl group, more preferably a vinyl group or a propenyl group, and particularly preferably a vinyl group.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the above, R¹⁰¹ represents preferably a cyclic group which may have a substituent and more preferably a cyclic hydrocarbon group which may have a substituent. More specifically, the cyclic hydrocarbon group is preferably a group obtained by removing one or more hydrogen atoms from a phenyl group, a naphthyl group, or a polycycloalkane; a lactone-containing cyclic group represented by any of General Formulae (a2-r-1) to (a2-r-7); or a —SO₂-containing cyclic group represented by any of General Formulae (a5-r-1) to (a5-r-4), more preferably a group obtained by removing one or more hydrogen atoms from a polycycloalkane; or a —SO₂-containing cyclic group represented by any of General Formulae (a5-r-1) to (a5-r-4), and still more preferably an adamantyl group or a —SO₂-containing cyclic group represented by General Formula (a5-r-1).

In a case where the cyclic hydrocarbon group has a substituent, the substituent is preferably a hydroxyl group.

In Formula (b-1), Y¹⁰¹ represents a single bond or a divalent linking group having an oxygen atom.

In a case where Y¹⁰¹ represents a divalent linking group containing an oxygen atom, Y¹⁰¹ may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. A sulfonyl group (—SO₂—) may be further linked to the combination. Examples of the divalent linking group having an oxygen atom include linking groups each represented by General Formulae (y-al-1) to (y-al-7). In General Formulae (y-al-1) to (y-al-7), V′¹⁰¹ in General Formulae (y-al-1) to (y-al-7) is bonded to R¹⁰¹ in Formula (b-1).

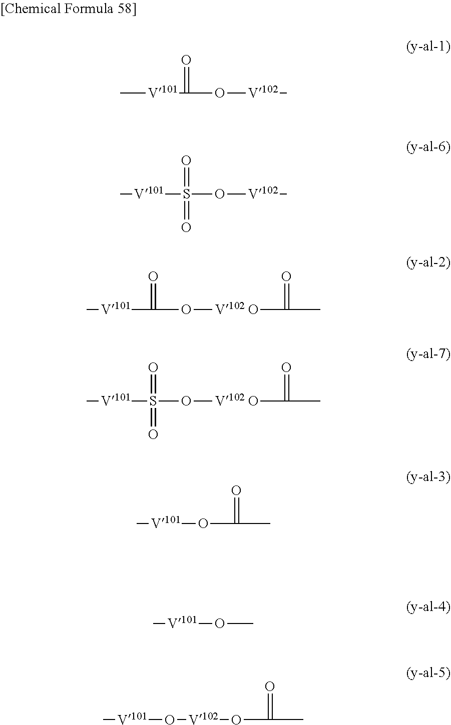

[In the formulae, V′¹⁰¹ represents a single bond or an alkylene group having 1 to 5 carbon atoms, and V′¹⁰² represents a divalent saturated hydrocarbon group having 1 to 30 carbon atoms.]

The divalent saturated hydrocarbon group as V′¹⁰² is preferably an alkylene group having 1 to 30 carbon atoms, more preferably an alkylene group having 1 to 10 carbon atoms, and still more preferably an alkylene group having 1 to 5 carbon atoms.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

In addition, a part of methylene group in the alkylene group as V′¹⁰¹ and V′¹⁰² may be substituted with a divalent aliphatic cyclic group having 5 to 10 carbon atoms. The aliphatic cyclic group is preferably a divalent group obtained by removing one hydrogen atom from the cyclic aliphatic hydrocarbon group (a monocyclic aliphatic hydrocarbon group or a polycyclic aliphatic hydrocarbon group) as Ra′³ in General Formula (a1-r-1), and more preferably a cyclohexylene group, a 1,5-adamantylene group, or a 2,6-adamantylene group.

Y¹⁰¹ represents preferably a divalent linking group having an ester bond or a divalent linking group having an ether bond and more preferably a linking group represented by any of Formulae (y-al-1) to (y-al-5).

In Formula (b-1), V¹⁰¹ represents a single bond, an alkylene group, or a fluorinated alkylene group. It is preferable that the alkylene group and the fluorinated alkylene group as V¹⁰¹ have 1 to 4 carbon atoms. Examples of the fluorinated alkylene group as V¹⁰¹ include a group in which some or all hydrogen atoms in the alkylene group as V¹⁰¹ have been substituted with fluorine atoms. Among these, it is preferable that V¹⁰¹ represents a single bond or a fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

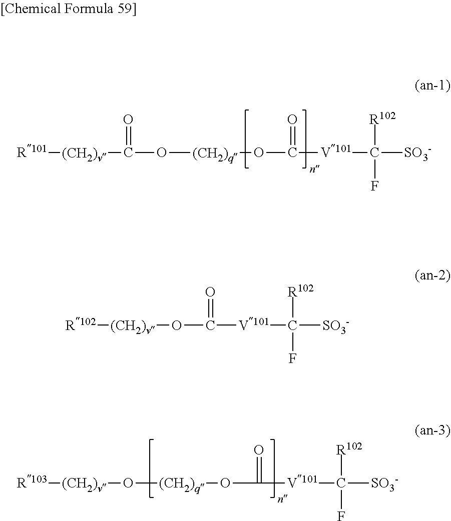

[In the formulae, R″¹⁰¹ represents an aliphatic cyclic group which may have a substituent, a monovalent heterocyclic group represented by any of Chemical Formulae (r-hr-1) to (r-hr-6), a condensed cyclic group represented by Formula (r-br-1) or (r-br-2), or a chain-like alkyl group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (a5-r-1) to (a5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.1

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group as R″¹⁰³ which may have a substituent, the same groups as those for the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anion in Component (b-2)

In Formula (b-2), R′¹⁰⁴ and R′¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Provided that R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms from the viewpoint that the solubility in a solvent for a resist is also satisfactory. In addition, in the chain-like alkyl group as R¹⁰⁴ and R¹⁰⁵, it is preferable that the number of hydrogen atoms substituted with fluorine atoms is as large as possible from the viewpoint that the acid strength increases and the transparency to high energy light or electron beams having a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V^{L01} in Formula (b-1).

In Formula (b-2), L¹⁰¹ and L¹⁰² each independently represent a single bond or an oxygen atom.

Anion in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for R¹⁰¹ in Formula (b-1).

In Formula (b-3), L¹⁰³ to L¹⁰⁵ each independently represent a single bond, —CO—, or —SO₂—.

Among the above, as the anion moiety of the component (B), an anion in the component (b-1) is preferable. Among these, an anion represented by any of General Formulae (an-1) to (an-3) is more preferable, an anion represented by General Formula (an-1) or (an-2) is still more preferable, and an anion represented by General Formula (an-2) is particularly preferable.

### {Cation Moiety}

In Formulae (b-1), (b-2), and (b-3), M⁺ represents an m-valent onium cation and is the same as M^{m+} in General Formula (a0). Among these, a sulfonium cation and an iodonium cation are preferable.

m represents an integer of 1 or more.

Preferred examples of the cation moiety ((M^{m+})_{l/m}) include organic cations each represented by General Formulae (ca-1) to (ca-5).

Among the examples, a cation represented by General Formula (ca-1) is preferable as the cation moiety ((M^{m+})_{l/m}).

In the resist composition according to the present embodiment, the component (B) may be used alone or in combination of two or more kinds thereof.

The content of the component (B) in the resist composition according to the present embodiment is preferably less than 40 parts by mass, more preferably in a range of 1 to 30 parts by mass, and still more preferably in a range of 3 to 25 parts by mass with respect to 100 parts by mass of the component (A).

In a case where the content of the component (B) is set to be in the above-described preferable range, pattern formation can be satisfactorily performed. In addition, it is preferable that each component of the resist composition is dissolved in an organic solvent from the viewpoint that a uniform solution is easily obtained and the storage stability of the resist composition is improved.

Provided that, from the viewpoint of improving lithography characteristics such as LWR, the resist composition according to the present embodiment preferably does not contain the component (B).

### <Other Components>

The resist composition according to the present embodiment may further contain other components in addition to the component (A) and the component (B) described above. Examples of other components include a component (D), a component (E), a component (F), and a component (S) described below.

### <<Base Component (D)>>

It is preferable that the resist composition according to the present embodiment further contains a base component (hereinafter, also referred to as “component (D)”) that traps (that is, controls the acid diffusion) an acid that is generated upon exposure to light, in addition to the component (A) and the component (B). The component (D) acts as a quencher (an acid diffusion control agent) which traps the acid generated in the resist composition upon exposure to light.

Examples of the component (D) include a photodecomposable base (D1) having an acid diffusion controllability (hereinafter, referred to as “component (D1)”) which is lost by the decomposition upon exposure to light and a nitrogen-containing organic compound (D2) (hereinafter, referred to as “component (D2)”) which does not correspond to the component (DI). Among these, the photodecomposable base (the component (D1)) is preferable since it is easy to enhance the roughness reducing property. In addition, in a case where the component (D1) is contained, both the characteristics of increasing the sensitivity and suppressing the occurrence of coating defects are likely to be enhanced.

### In Regard to Component (D1)

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon exposure to light and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film.

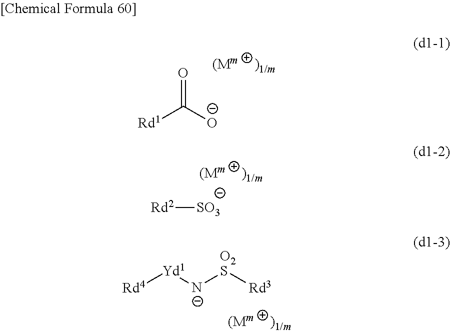

[In the formulae, Rd¹ to Rd⁴ represent a cyclic group which may have a substituent, a chain-like alkyl group which may have a substituent, or a chain-like alkenyl group which may have a substituent. Provided that no fluorine atom is bonded to the carbon atom adjacent to the S atom in Rd² of Formula (d1-2). Yd¹ represents a single bond or a divalent linking group. m represents an integer of 1 or more, and M^{m+}'s each independently represent an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-al-1) to (y-al-5) is preferable as the substituent. In a case where the aromatic hydrocarbon group, the aliphatic cyclic group, or the chain-like alkyl group as Rd¹ contain a linking group represented by any of General Formulae (y-al-1) to (y-al-7) as a substituent, V′¹⁰¹ in General Formulae (y-al-1) to (y-al-7) is bonded to the carbon atom constituting the aromatic hydrocarbon group, the aliphatic cyclic group, or the chain-like alkyl group as Rd¹ in Formula (d3-1), in General Formulae (y-al-1) to (y-al-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

The aliphatic cyclic group is more preferably a group obtained by removing one or more hydrogen atoms from polycycloalkanes such as adamantane, norbornane, isobornane, tricyclodecane, and tetracyclododecane.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

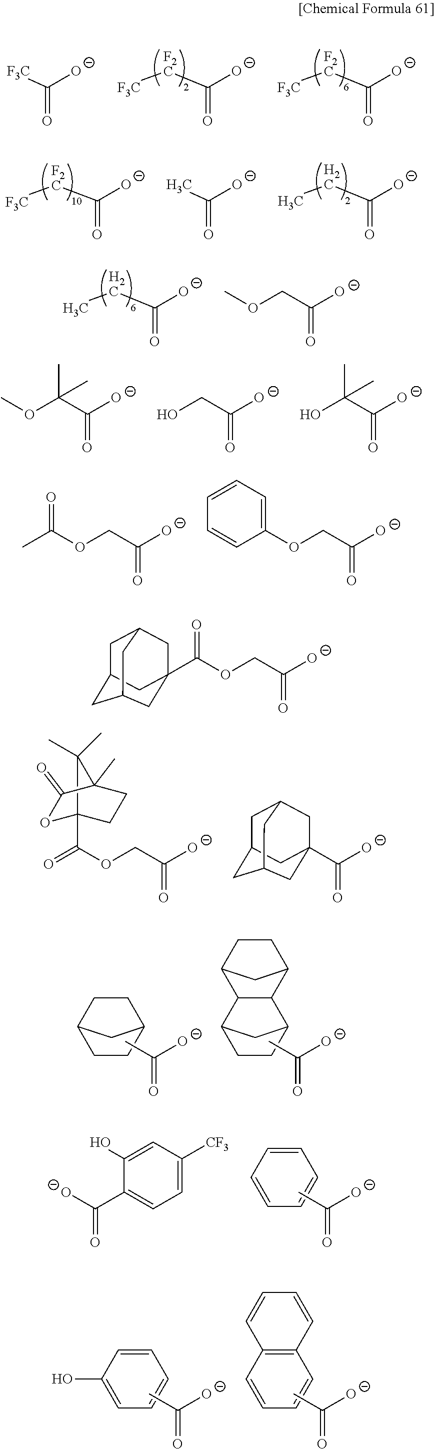

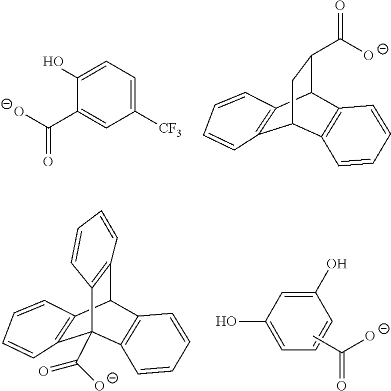

### Cation Moiety

In Formula (d1-1), M^{m+} represents an m-valent organic cation.

The suitable examples of the organic cation as M^{m+} include the same cations as the cations each represented by General Formulae (ca-1) to (ca-3), the cation represented by General Formula (ca-1) is preferable, and cations each represented by General Formulae (ca-1-1) to (ca-1-113) are more preferable.

The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Provided that no fluorine atom is bonded to the carbon atom adjacent to the S atom in Rd² (the carbon atom is not substituted with fluorine). In this manner, the anion of the component (d1-2) is an appropriately weak acid anion, thereby improving the quenching ability of the component (D).

Rd² represents preferably a chain-like alkyl group which may have a substituent or an aliphatic cyclic group which may have a substituent and more preferably an aliphatic cyclic group which may have a substituent.

The chain-like alkyl group has preferably 1 to 10 carbon atoms and more preferably 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent, and examples of the substituent include the same groups as those for the substituent which may be contained in the hydrocarbon group (such as an aromatic hydrocarbon group, an aliphatic cyclic group, or a chain-like alkyl group) as Rd¹ in General Formula (d1-1).

Among the above, the anion moiety of the component (d1-2) is preferably a camphorsulfonic acid anion.

Specific preferred examples of the anion moiety in the component (d1-2) are described below.

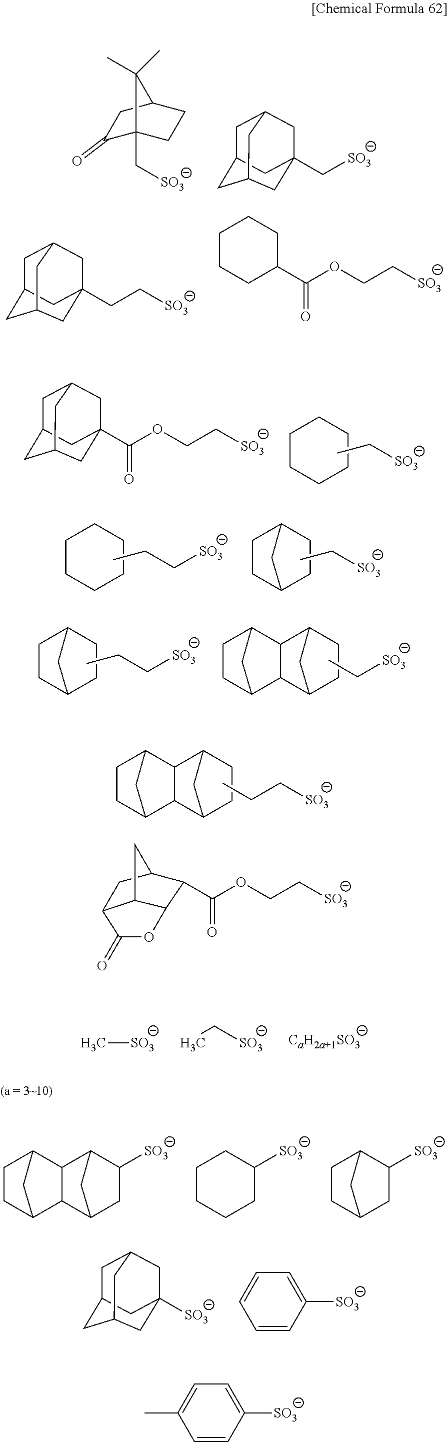

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may further contain an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. In addition, in a case where Rd⁴ represents an aromatic group, the resist composition has an excellent light absorption efficiency in lithography using EUV or the like as a light source for exposure, and thus the sensitivity and lithography characteristics are enhanced.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a hetero atom. Examples of the divalent linking groups are the same as those for the divalent hydrocarbon group which may have a substituent and the divalent linking group having a hetero atom described in the section of the divalent linking group as Ya²¹ in Formula (a2-1).

It is preferable that Yd¹ represents a carbonyl group, an ester bond, an amide bond, an alkylene group, or a combination thereof. The alkylene group is more preferably a linear or branched alkylene group, and still more preferably a methylene group or an ethylene group.

Specific preferred examples of the anion moiety in the component (d1-3) are described below.

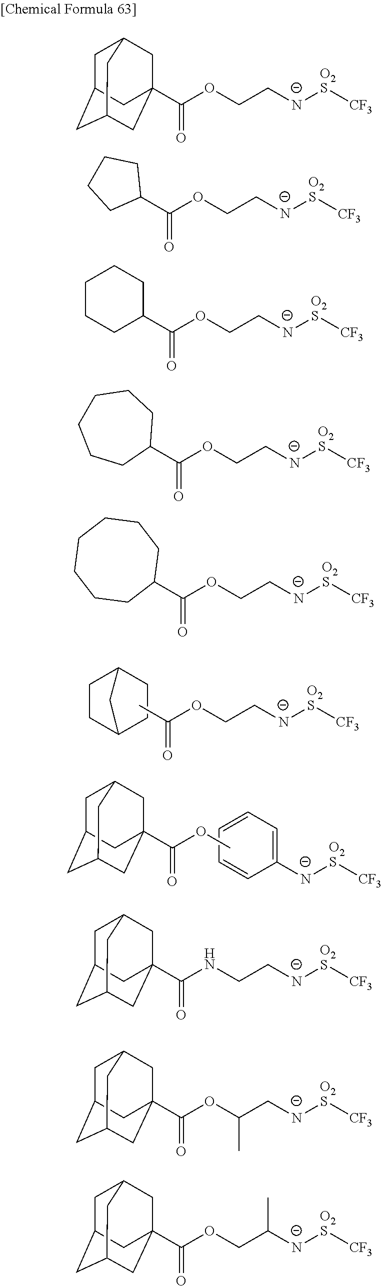

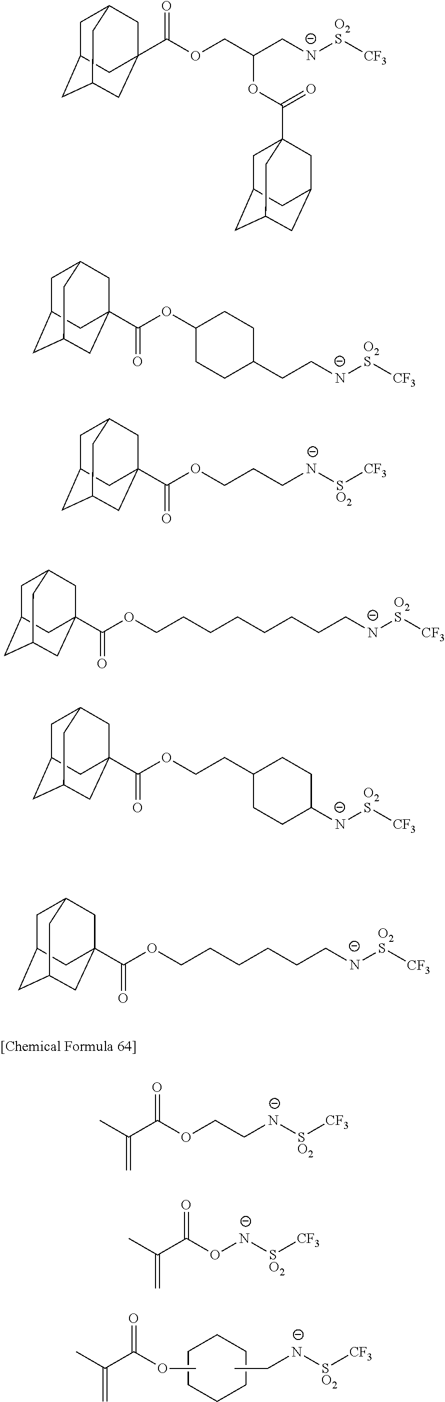

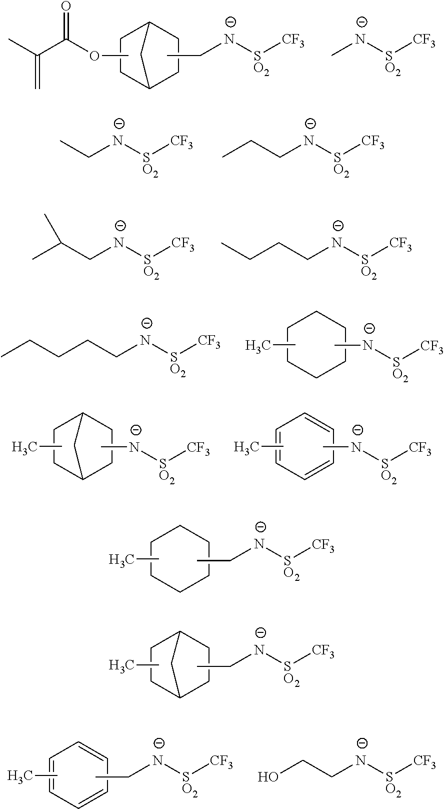

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 3 to 10 parts by mass with respect to 100 parts by mass of the component (A1).

In a case where the content of the component (D1) is equal to or more than the lower limit values of the above-described preferable ranges, excellent lithography characteristics and an excellent resist pattern shape are easily obtained. On the other hand, in a case where the content thereof is equal to or less than the upper limit values, the sensitivity can be maintained satisfactorily and the throughput is also excellent.

In the resist composition according to the present embodiment, it is preferable that the component (D1) contains the component (d1-1).

The content of the component (d1-1) in the total component (D) contained in the resist composition according to the present embodiment is preferably 50% by mass or more, preferably 70% by mass or more, and still more preferably 90% by mass or more, and the component (D) may consist of only the component (d1-1).

### Method for Producing Component (D1):

The methods for producing the component (d1-1) and the component (d1-2) are not particularly limited, and these components can be produced by known methods.

In addition, the method for producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and an optional component may be selected from known components and then used. Among these, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH₃ has been substituted with an alkyl group or hydroxyalkyl group having 12 or less carbon atoms (alkyl amines or alkyl alcohol amines), and cyclic amines.

Specific examples of the alkylamines and the alkylalcoholamines include monoalkylamines such as n-hexylamine. n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is preferable, and tri-n-pentylamine and tri-n-octylamine are particularly preferable.

Examples of the cyclic amine include a heterocyclic compound having a nitrogen atom as a hetero atom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

It is preferable that the aliphatic polycyclic amine has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

In addition, as the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

Among the above, the component (D2) is preferably an alkyl amine and more preferably a trialkyl amine having 5 to 10 carbon atoms.

The component (D2) may be used alone or in combination of two or more kinds thereof.

In a case where the resist composition contains the component (D2), the content of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A1).

In a case where the content of the component (D2) is equal to or more than the lower limit values of the above-described preferable ranges, excellent lithography characteristics and an excellent resist pattern shape are easily obtained. On the other hand, in a case where the content thereof is equal to or less than the upper limit values, the sensitivity can be maintained satisfactorily and the throughput is also excellent.

<<At Least One Compound (E) Selected from Group Consisting of Organic Carboxylic Acids, and Phosphorus Oxo Acids and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group.

Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition according to the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). Within the above range, the lithography characteristics are further improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), it is possible to use, for example, a fluorine-containing polymer compound described in Japanese Unexamined Patent Application, First Publication No. 2010-002870, Japanese Unexamined Patent Application, First Publication No. 2010-032994. Japanese Unexamined Patent Application, First Publication No. 2010-277043, Japanese Unexamined Patent Application, First Publication No. 2011-13569, or Japanese Unexamined Patent Application, First Publication No. 2011-128226.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

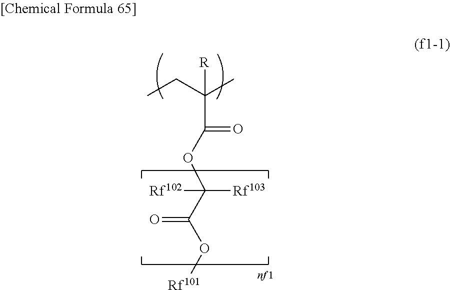

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf⁴⁰² and Rf¹⁰³ may be the same as or different from each other. nf¹ represents an integer of 0 to 5, and R″¹ represents an organic group having a fluorine atom.]

In Formula (f1-1), R bonded to the carbon atom at the α-position has the same definition as described above. R is preferably a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as R″¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or more of the hydrogen atoms in the hydrocarbon group are fluorinated, more preferably 50% or more thereof are fluorinated, and particularly preferably 60% or more thereof are fluorinated from the viewpoint of increasing the hydrophobicity of the resist film during immersion exposure.

Among these. Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1,000 to 50.000, more preferably in a range of 5,000 to 40.000, and most preferably in a range of 10.000 to 30,000. In a case where the weight-average molecular weight thereof is equal to or less than the upper limit values of the above-described ranges, the resist composition exhibits a sufficient solubility in a solvent for a resist enough to be used as a resist. Meanwhile, in a case where the weight-average molecular weight thereof is equal to or more than the lower limit values of the above-described ranges, water repellency of the resist film is satisfactory.

The dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component (S)>>

The resist composition according to the present embodiment can be produced by dissolving the resist materials in an organic solvent component (hereinafter, referred to as “component (S)”).

The component (S) may be any organic solvent which can dissolve each component to be used to obtain a uniform solution, and an optional organic solvent can be appropriately selected from those which have been known as solvents of a chemically amplified resist composition and then used in the related art.

Examples of the component (S) include lactones such as γ-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond, such as ethylene glycol monoacetate, diethylene glycol monoacetate, propylene glycol monoacetate, and dipropylene glycol monoacetate; polyhydric alcohol derivatives of compounds having an ether bond such as monoalkyl ether or monophenyl ether, such as monomethylether, monoethylether, monopropylether, or monobutylether of polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene, and mesitylene; and dimethylsulfoxide (DMSO).

In the resist composition according to the present embodiment, the component (S) may be used alone or in the form of a mixed solvent of two or more kinds thereof. Among these, PGMEA, PGME, γ-butyrolactone, EL, or cyclohexanone is preferable.

In addition, a mixed solvent obtained by mixing PGMEA with a polar solvent is also preferable as the component (S). The blending ratio (mass ratio) of the mixed solvent can be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. In addition, in a case where PGME is blended as the polar solvent, a mass ratio of PGMEA:PGME is preferably 1:9 to 9:1, more preferably 2:8 to 8:2, and still more preferably 3:7 to 7:3. Furthermore, a mixed solvent of PGMEA, PGME, and cyclohexanone is also preferable.

In addition, the component (S) is also preferably a mixed solvent of at least one selected from PGMEA and EL and γ-butyrolactone. In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition according to the present embodiment, as appropriate.

After the resist material is dissolved in the component (S), impurities may be removed from the resist composition according to the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains the resin component (A1) having the constitutional unit (a0) derived from the compound represented by General Formula (a0). In the constitutional unit (a0), a terminal —C(Ra⁰¹)(Ra⁰²)—SO₃⁻(M^{m+})_{l/m} of the side chain in General Formula (a0) functions as an acid generator. In addition, since the hydroxy group (—OH) bonded to Ar in General Formula (a0) is a phenolic hydroxyl group, the constitutional unit (a0) functions as a proton source. Since the acid generator group —C(Ra⁰¹)(Ra⁰²)—SO₃⁻(M^{m+})_{l/m} and the proton source (the hydroxy group (—OH) bonded to Ar) coexist in the constitutional unit (a0), it is presumed that the proton generated from the proton source is reliably reacted with the acid generator group, contributing to the improvement in sensitivity.

In addition, since the acid generator group —C(Ra⁰¹)(Ra⁰²)—SO₃⁻(M^{m+})_{l/m} and the proton source (the hydroxy group (—OH) bonded to Ar) coexist in the constitutional unit (a0), it is presumed that the in-film uniformity is improved, contributing to the improvement in the lithography characteristics such as LWR.

In conjunction with the above-described effects, it is presumed that, according to the resist composition of the present embodiment, a resist pattern capable of increasing the sensitivity and having satisfactory lithography characteristics such as LWR can be formed.

### (Method for Forming Resist Pattern)

A method for forming a resist pattern according to the second aspect according to the present invention is a method including a step of forming a resist film on a support using the resist composition according to the first aspect of the present invention described above, a step of exposing the resist film to light, and a step of developing the resist film exposed to light to form a resist pattern.

According to the embodiment of the method for forming a resist pattern, a method for forming a resist pattern by performing processes as described below is an exemplary example.

First, a support is coated with the resist composition of the present embodiment using a spinner or the like, and a bake (post applied bake (PAB)) treatment is performed under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing with an electron beam without using a mask pattern, baking treatment (post-exposure baking (PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse liquid containing an organic solvent is preferable in a case of the solvent developing process.

In a case of the solvent developing process, after the developing treatment or the rinse treatment, the developing solution or the rinse liquid attached onto the pattern may be removed by a treatment using a supercritical fluid.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

In addition, as the support, any one of the above-described supports provided with an inorganic and/or organic film on the above-described substrate may be used. As the inorganic film, an inorganic antireflection film (inorganic BARC) can be used. As the organic film, an organic film such as an organic antireflection film (organic BARC) or a lower-layer organic film used in a multilayer resist method can be used.

Here, the multilayer resist method is a method of providing at least one layer of an organic film (lower-layer organic film) and at least one layer of a resist film (upper-layer resist film) on a substrate and performing patterning of the lower-layer organic film using a resist pattern formed on the upper-layer resist film as a mask, and this method is considered to enable formation of a pattern with a high aspect ratio. That is, according to the multilayer resist method, since a desired thickness can be ensured by the lower-layer organic film, the thickness of the resist film can be reduced, and a fine pattern with a high aspect ratio can be formed.

The multilayer resist method is basically classified into a method in which a double-layer structure consisting of an upper-layer resist film and a lower-layer organic film is formed (double-layer resist method), and a method in which a multilayer structure having three or more layers consisting of an upper-layer resist film, a lower-layer organic film, and one or more intermediate layers (thin metal films or the like) provided between the upper-layer resist film and the lower-layer organic film (triple-layer resist method).

The wavelength to be used for exposure is not particularly limited and the exposure can be conducted using radiation such as an ArF excimer laser, a KrF excimer laser, an F² excimer laser, an extreme ultraviolet ray (EUV), a vacuum ultraviolet ray (VUV), an electron beam (EB), an X-ray, and a soft X-ray. The resist composition is useful for a KrF excimer laser, an ArF excimer laser, EB, and EUV, more useful for an ArF excimer laser, EB, and EUV, and particularly useful for EB and EUV. That is, the method for forming a resist pattern according to the present embodiment is a method particularly useful in a case where the step of exposing the resist film to light includes a process of exposing the resist film to extreme ultraviolet rays (EUV) or electron beams (EB).

The exposure method of the resist film can be a typical exposure (dry exposure) carried out in air or an inert gas such as nitrogen, or liquid immersion lithography; however, liquid immersion lithography is more preferable.

The liquid immersion lithography is an exposure method in which the region between the resist film and the lens at the lowermost position of the exposure apparatus is filled with a solvent (liquid immersion medium) in advance that has a larger refractive index than the refractive index of air, and the exposure (immersion exposure) is conducted in this state.

As the liquid immersion medium, a solvent which has a refractive index greater than the refractive index of air but less than the refractive index of the resist film to be exposed to light is preferable. The refractive index of such a solvent is not particularly limited as long as the refractive index is in the above-described range.

Examples of the solvent which has a refractive index that is greater than the refractive index of air but less than the refractive index of the resist film include water, a fluorine-based inert liquid, a silicon-based solvent, and a hydrocarbon-based solvent.

Specific examples of the fluorine-based inert liquid include a liquid containing a fluorine-based compound such as C₃HCl₂FS, C₄F₉OCH₃, C₄F₉OC₂H₅, or C₅H₃F₇ as a main component, and a liquid with a boiling point of 70° C. to 180° C. is preferable and a liquid with a boiling point of 80° C. to 160° C. is more preferable. A fluorine-based inert liquid having a boiling point in the above-described range is preferable from the viewpoint that a medium used for liquid immersion can be removed using a simple method after completion of light exposure.

The fluorine-based inert liquid is particularly preferably a perfluoroalkyl compound in which all of the hydrogen atoms of the alkyl group are substituted with a fluorine atom. Specific examples of the perfluoroalkyl compound include a perfluoroalkylether compound and a perfluoroalkylamine compound.

Furthermore, specific examples of the perfluoroalkylether compound include perfluoro(2-butyl-tetrahydrofuran) (boiling point of 102° C.), and specific examples of the perfluoroalkylamine compound include perfluorotributylamine (boiling point of 174° C.).

As the liquid immersion medium, water is preferable from the viewpoints of the cost, the safety, the environmental issues, and the versatility.

As the alkali developing solution used for the developing treatment in the alkali developing process, a 0.1 to 10% by mass tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

The ketone-based solvent is an organic solvent containing C—C(═O)—C in the structure thereof. The ester-based solvent is an organic solvent containing C—C(═O)—O—C in the structure thereof. The alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group.

The nitrile-based solvent is an organic solvent containing a nitrile group in the structure thereof. The amide-based solvent is an organic solvent containing an amide group in the structure thereof. The ether-based solvent is an organic solvent containing C—O—C in the structure thereof.

As the organic solvent, an organic solvent having a plurality of the functional groups which characterize the above-described solvents in the structure thereof is also present. In such a case, the organic solvent can be classified as any type of solvent having a functional group that characterizes a solvent. For example, diethylene glycol monomethylether corresponds to both the alcohol-based solvent and the ether-based solvent which have been classified above.

The hydrocarbon-based solvent is a hydrocarbon solvent which is formed of a hydrocarbon that may be halogenated and does not have a substituent other than halogen atoms. The halogen atom is preferably a fluorine atom.

Among the examples, as the organic solvent contained in the organic developing solution, a polar solvent is preferable. Further, a ketone-based solvent, an ester-based solvent, and a nitrile-based solvent are preferable.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonylalcohol, acetylcarbinol, acetophenone, methyl naphthyl ketone, isophorone, propylenecarbonate, γ-butyrolactone, and methyl amyl ketone (2-heptanone). Among these, the ketone-based solvent is preferably methylamyl ketone (2-heptanone).

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate, and propyl-3-methoxypropionate. Among these, the ester-based solvent is preferably butyl acetate.

Examples of the nitrile-based solvent include acetonitrile, propionitrile, valeronitrile, and butyronitrile.

As desired, the organic developing solution may have a conventionally known additive blended. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be carried out by a conventionally known developing method. Examples thereof include a method in which the support is immersed in the developing solution for a predetermined time (a dip method), a method in which the developing solution is cast upon the surface of the support by surface tension and maintained for a predetermined time (a puddle method), a method in which the developing solution is sprayed onto the surface of the support (spray method), and a method in which a developing solution is continuously ejected from a developing solution ejecting nozzle and applied onto a support, which is being rotated at a constant rate while being scanned at a constant rate (dynamic dispense method).

As the organic solvent contained in the rinse liquid used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one kind of solvent selected from a hydrocarbon-based solvent, a ketone-based solvent, an ester-based solvent, an alcohol-based solvent, and an amide-based solvent is preferable, at least one kind of solvent selected from an alcohol-based solvent and an ester-based solvent is more preferable, and an alcohol-based solvent is particularly preferable.

As the alcohol-based solvent used in the rinse liquid, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or in combination of two or more kinds thereof. In addition, an organic solvent other than the above-described examples or water may be mixed thereto. However, in consideration of the development characteristics, the amount of water to be blended into the rinse liquid is preferably 30% by mass or less, more preferably 10% by mass or less, still more preferably 5% by mass or less, and particularly preferably 3% by mass or less with respect to the total amount of the rinse liquid.

A known additive can be blended into the rinse liquid as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse liquid.

The rinse treatment carried out using a rinse liquid (washing treatment) can be performed according to a known rinse method. Examples of the method of performing the rinse treatment include a method of continuously ejecting a rinse liquid onto a support rotating at a constant rate (rotary coating method), a method of immersing a support in a rinse liquid for a predetermined time (dip method), and a method of spraying a rinse liquid to the surface of a support (spray method).

According to the method for forming a resist pattern of the present embodiment described above, since the resist composition described above is used, a resist pattern with high sensitivity and satisfactory CDU can be formed.

It is preferable that various materials that are used in the resist composition according to the above-described embodiment and the method for forming a pattern according to the above-described embodiment (for example, a resist solvent, a developing solution, a rinse liquid, a composition for forming an antireflection film, and a composition for forming a top coat) do not contain impurities such as a metal, a metal salt containing halogen, an acid, an alkali, and a component having a sulfur atom or phosphorus atom. Here, examples of the impurities containing metal atoms include Na, K, Ca. Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, and particularly preferably 10 ppt or less, where it is most preferable that the impurities are substantially free (equal to or less than the detection limit of the measuring device).

### (Polymer Compound)

The polymer compound according to the third aspect of the present invention is a polymer compound having a constitutional unit (a0) derived from the compound represented by General Formula (a0).

The polymer compound according to the present embodiment is the same as the resin component (A1) described in the resist composition according to the first aspect.

The polymer compound according to the present embodiment can also be suitably used as a resin component of a resist composition.

### (Compound)

The compound according to the fourth aspect of the present invention is a compound represented by General Formula (a0) (hereinafter, also referred to as a “compound (am0)”).

The compound (am0) is the same as the compound from which the constitutional unit (a0) described in the resist composition according to the first aspect is derived.

As the compound (am0), the compound represented by General Formula (a0-1) is preferable.

Specific examples of the compound (am0) are shown below. In the formula, R⁰ is the same as described above. m and M^{m+} are the same as m and M^{m+} in General Formula (a0).

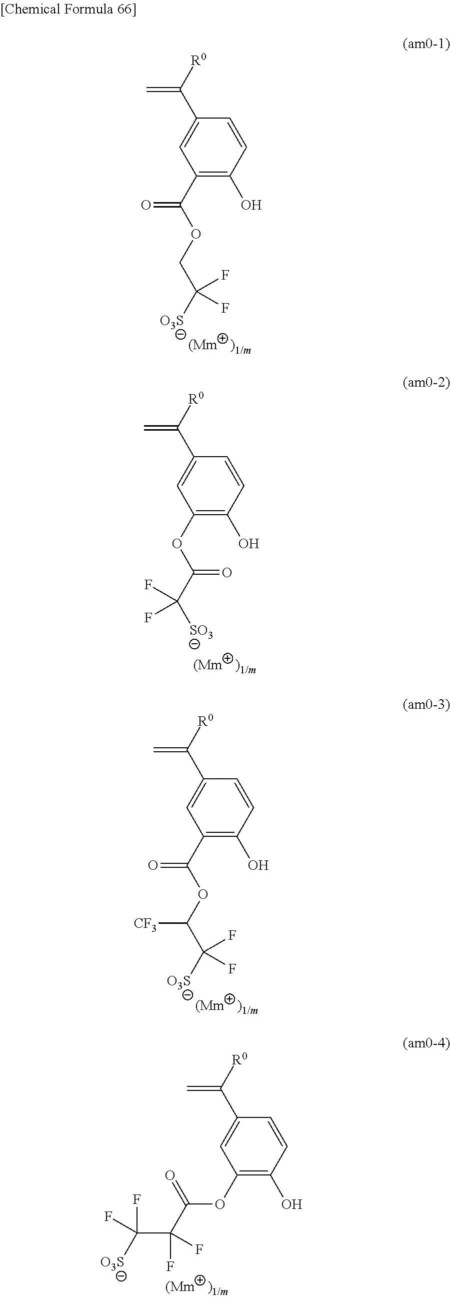

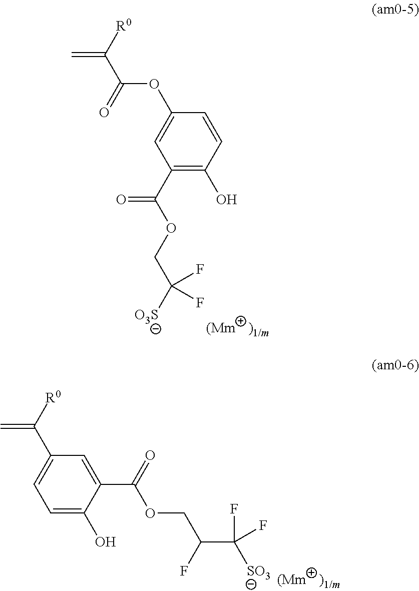

Among the above, the constitutional unit (a0) is preferably a constitutional unit represented by any one of General Formulae (am0-1) to (a0m-6).

### (Method for Producing Compound (Am0))

The compound (am0) according to the present embodiment may be produced by, for example, a method including the following step (A) and step (B) in a case where La⁰ in General Formula (a0) is —C(═O)—O— or —O—C(═O)—.

### [Step A]

The step A is a step of subjecting the compound represented by General Formula (C-1) (hereinafter, also referred to as “compound (C1)”) and the compound represented by General Formula (C-2) (hereinafter, also referred to as “compound (C2)”) to a condensation reaction to obtain a compound (am0op) represented by General Formula (am0-p) (hereinafter, also referred to as “compound (am0p)”).

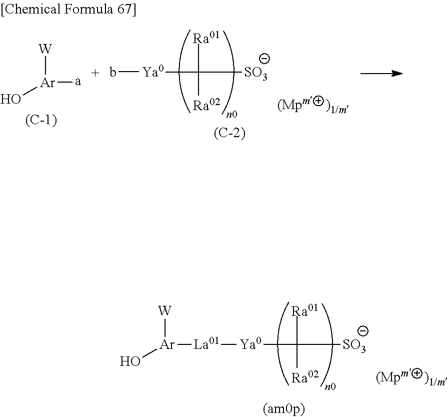

[In the formula, W represents a polymerizable group. Ar represents an aromatic hydrocarbon group which may have a substituent. —OH represents a hydroxy group. One of a and b represents a hydroxy group, and the other represents a carboxy group. Ya⁰ represents a single bond or a divalent linking group which may have a hetero atom. Ra⁰¹ and Ra⁰² each independently represent a hydrogen atom, a fluorine atom, or a fluorinated alkyl group. n0 represents an integer in a range of 1 to 4. m′ represents an integer of 1 or more, and M^{m′+} represents an m-valent organic ammonium cation or metal cation.]

In the formula, W, Ar, Ya⁰, Ra⁰¹, Ra⁰², and n0 are the same as W, Ar, Ya⁰, Ra⁰¹, Ra⁰², and n0 in General Formula (a0).

In General Formula (am0p), m′ represents an integer of 1 or more, and M^{m′+} represents an m-valent organic ammonium cation or a metal cation.

Examples of the metal cation include an alkali metal cation such as a sodium cation or a potassium cation, an alkaline earth metal cation such as a calcium cation, and the like.

Specific examples of the organic ammonium cation include a cation represented by General Formula (ca-p-1) and a cation represented by General Formula (ca-p-2).

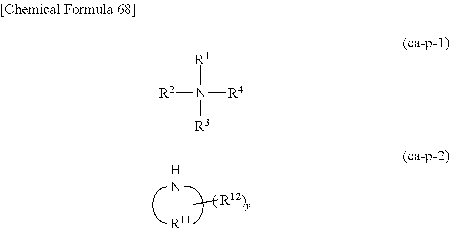

[In the formulae, R¹ to R⁴ each independently represent a hydrocarbon group which may have a substituent or a hydrogen atom. Provided that at least one of R¹ to R⁴ represents a hydrocarbon group which may have a substituent. R″ represents a group that forms an aromatic ring together with the nitrogen atom to which R″ is bonded, R¹² represents an alkyl group or a halogen atom, and y represents an integer of 0 to 5.]

In General Formula (ca-p-1), R¹ to R⁴ each independently represent a hydrocarbon group which may have a substituent or a hydrogen atom. The hydrocarbon groups as R¹ to R⁴ are each independently preferably a hydrocarbon group having 1 to 15 carbon atoms and more preferably a hydrocarbon group having 1 to 10 carbon atoms. In addition, the total number of carbon atoms in the hydrocarbon group as R¹ to R⁴ is preferably in a range of 1 to 20, more preferably in a range of 3 to 18, and still more preferably in a range of 4 to 15.

Examples of the hydrocarbon group include a linear or branched alkyl group, a cyclic hydrocarbon group, or the like.

The linear or branched alkyl group is preferably a linear or branched alkyl group having 1 to 10 carbon atoms and more preferably a linear or branched alkyl group having 1 to 10 carbon atoms.

The cyclic hydrocarbon group may be an aliphatic hydrocarbon group or an aromatic hydrocarbon group.

As the cyclic aliphatic hydrocarbon group, a group in which one hydrogen atom has been removed from a monocycloalkane is preferable. The monocycloalkane has preferably 3 to 6 carbon atoms, and specific examples thereof include cyclopentane and cyclohexane.

As the cyclic aromatic hydrocarbon group, a phenyl group is preferable.

Examples of the substituent which may be included in the hydrocarbon group as R¹ to R⁴ include an alkyl group, an alkoxy group, a hydroxyl group, an oxo group (═O), and an amino group.

In General Formula (ca-p-2), R¹¹ represents a group that forms an aromatic ring together with the nitrogen atom to which the R¹¹ is bonded. The aromatic ring is preferably a 4- to 7-membered ring, more preferably a 4- to 6-membered ring, and still more preferably a 6-membered ring.

In General Formula (ca-p-2), R¹² represents an alkyl group, and examples thereof include the same groups as those for the linear or branched alkyl group as R¹ to R⁴.

In General Formula (ca-p-2), y represents an integer of 0 to 5, preferably 1 or 0, and more preferably 0.

The condensation reaction in the step A may be carried out in the presence of a condensing agent and a base catalyst (additive).

Specific examples of the condensing agent include 1,1′-carbonyldiimidazole, N,N′-dicyclohexylcarbodiimide, N,N′-diisopropylcarbodiimide (DIC), 1-ethyl-3-(3-dimethylaminopropyl)carbodiimide hydrochloride, and carbonyldiimidazole (CDI).

Specific examples of the base catalyst include tertiary amines such as trimethylamine, triethylamine, and tributylamine, aromatic amines such as pyridine, pyrrolidinopyridine, and 4-(dimethylamino)pyridine (DMAP), diazabicyclononene (DBN), and diazabicycloundecene (DBU).

In addition, the condensation reaction in the step A may be carried out in the presence of an acid catalyst.

Specific examples of the acid catalyst include diphosphorus pentoxide and methanesulfonic acid.

The reaction time of the step A is, for example, preferably 5 minutes or more and 24 hours or less, more preferably 10 minutes or more and 10 hours or less, and still more preferably 10 minutes or more and 5 hours or less.

The reaction temperature of the step A is preferably in a range of 0° C. to 80° C. and more preferably in a range of 10° C. to 70° C.

Examples of the reaction solvent in the step A include dichloromethane, dichloroethane, chloroform, diethyl ether, tetrahydrofuran, N,N-dimethylformamide, acetonitrile, propionitrile, N,N′-dimethylacetamide, and dimethyl sulfoxide.

After the completion of the condensation reaction, the compound in the reaction solution may be isolated and purified. A known method in the related art can be used for isolation and purification, and for example, concentration, solvent extraction, distillation, crystallization, recrystallization, or chromatography can be appropriately combined and used.

### <Step B>

The step B is a step of subjecting the compound (am0p) described above to an ion exchange reaction with the compound represented by General Formula (C-3) to obtain a compound (am0) represented by General Formula (am01-1) (hereinafter, also referred to as a “compound (am01-1)”).

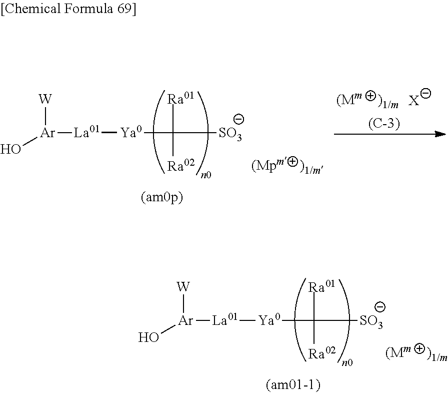

[In the formula, W represents a polymerizable group. Ar represents an aromatic hydrocarbon group which may have a substituent. —OH represents a hydroxy group. La⁰¹ represents —C(═O)—O— or —O—C(═O). Ya⁰ represents a single bond or a divalent linking group which may have a hetero atom. Ra⁰¹ and Ra⁰² each independently represent a hydrogen atom, a fluorine atom, or a fluorinated alkyl group. n0 represents an integer in a range of 1 to 4. m′ represents an integer of 1 or more, and M^{m′+} represents an m-valent organic ammonium cation or metal cation. m represents an integer of 1 or more, and M^{m+} represents an m-valent organic cation.]

In the formula described above, W, Ar, Ya⁰, Ra⁰¹, Ra⁰², n0, m, and M^{m+} are the same as W, Ar, Ya⁰, Ra⁰¹, Ra⁰², n0, m, and M^{m+} in General Formula (a0).

In the formula described above, m′ and M^{m′+} are the same as m′ and M^{m′+} in General Formula (am0p).

In General Formula (C-3), X⁻ represents a counter anion. Examples of X-include ions that can be an acid having a lower acidity than that of the compound (am0p), and specific examples thereof include a halogen ion such as a bromine ion or a chloride ion. BF₄⁻, AsF₆⁻, SbF₆⁻, PF₆⁻, ClO₄⁻, and the like.

The reaction time of the step B is, for example, preferably 0.5 minutes or more and 24 hours or less, more preferably 5 minutes or more and 12 hours or less, and still more preferably 10 minutes or more and 3 hours or less.

The reaction temperature of the step B is preferably in a range of 0° C. to 50° C. and more preferably in a range of 10° C. to 30° C.

As the reaction solvent in the step B, for example, a mixed solvent of an organic solvent and water is preferable. Examples of the organic solvent include a ketone-based solvent such as cyclohexanone, methyl ethyl ketone, or diethyl ketone, an ether-based solvent such as diethyl ether, t-butyl methyl ether, or diisopropyl ether, a halogen-based solvent such as tetrahydrofuran, 1,3-dioxolane, dichloromethane (methylene chloride), or 1,2-dichloroethane, an ester-based solvent such as ethyl acetate or propylene glycol monomethyl ether acetate, propionitrile, and a mixed solvent thereof.

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), an elemental analysis method, and an X-ray crystal diffraction method.

As the raw material that is used in each step, a commercially available raw material may be used, or a synthetic material may be used.

The compound according to the present embodiment is useful for producing the polymer compound of the third aspect.

### EXAMPLES

Hereinafter, the present invention will be described in more detail with reference to examples, but the present invention is not limited to these examples.

### Synthesis Example 1A: Synthesis of Compound (m01)

### (Synthesis of Compound (Pre-m01))

10.0 g of 5-ethenyl-2-hydroxybenzoic acid and 40.0 g of tetrahydrofuran were charged into a 300 mL four-necked flask at room temperature, and the mixture was stirred and dissolved. 24.7 g of 1,1′-carbonyldiimidazole was charged into the four-necked flask and stirred. 24.7 g of sulfonate and 11.6 g of diazabicycloundecene were dissolved in 55.3 g of tetrahydrofuran, and the solution was charged into the four-necked flask and stirred. The temperature was raised to 60° C. in a water bath, and aging was performed for 4 hours. The mixture was cooled to 30° C. in an ice bath. 80.0 g of ion-exchanged water and 80.0 g of methylene chloride were charged into the four-necked flask and stirred. After stopping the stirring, the mixture was transferred to a separatory funnel, and the aqueous layer was discharged. Furthermore, washing with 80.0 g of ion exchange was performed. The organic layer was concentrated with a rotary evaporator to obtain 23.7 g of a compound (pre-m01) of an oily substance.

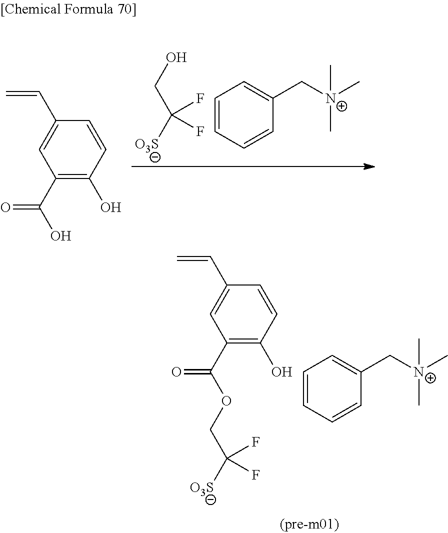

(Synthesis of Compound (m01))

10.0 g of the compound (pre-m01), 65.3 g of a 10 wt % triphenylphosphine chloride aqueous solution, and 100 g of methylene chloride were charged into a 300 mL beaker at room temperature, and the mixture was stirred for 2 hours. The reaction solution was transferred to a separatory funnel, and the aqueous layer was removed. The organic layer was washed three times with 56.1 g of ion-exchanged water. The organic layer was concentrated with a rotary evaporator to obtain 11.2 g of a compound (m01) of white powder.

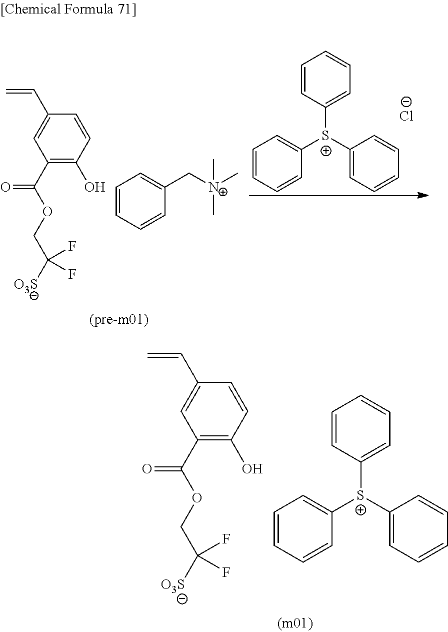

The obtained compound (m01) was subjected to NMR measurement, and the structure thereof was identified from the following analysis results.

Compound (m01)

¹H-NMR (DMSO-d6, 400 MHz): δ (ppm)=4.60 m, 5.25 m, 5.76 m, 6.72 m, 6.87 s, 7.17 d, 7.33 m, 7.66 m, 15.2 s

### Synthesis Example 2A: Synthesis of Compound (m02)

### (Synthesis of Compound (Pre-m02))

30.0 g of 4-ethyl-1,2-benzenediol and 120.0 g of tetrahydrofuran were charged into a 1,000 mL four-necked flask at room temperature, and the mixture was stirred and dissolved. 37.5 g of 1,1′-carbonyldiimidazole was charged into the four-necked flask and stirred. 68.1 g of sulfonate and 38.6 g of diazabicycloundecene were dissolved in 300 g of tetrahydrofuran, and the solution was charged into the four-necked flask and stirred. Subsequently, the temperature was raised to 60° C. in a water bath, and aging was performed for 4 hours. Thereafter, the mixture was cooled to 30° C. in an ice bath. 240.0 g of ion-exchanged water and 240.0 g of methylene chloride were charged into the four-necked flask and stirred. After stopping the stirring, the mixture was transferred to a separatory funnel, and the aqueous layer was discharged. Furthermore, washing with 240.0 g of ion exchange was performed. The organic layer was concentrated with a rotary evaporator to obtain 68.4 g of an oily substance. Subsequently, the oily substance was dissolved in 205.2 g of methanol, and the solution was cooled to obtain 19.5 g of a compound (pre-m02) of a white crystal.

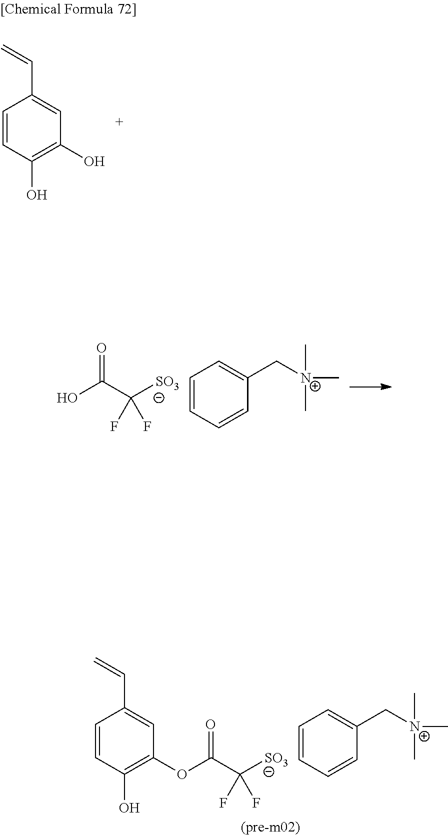

(Synthesis of Compound (m02))

A salt exchange reaction was performed in the same manner as in the synthesis of the compound (m01), except that the compound (pre-m02) was used instead of the compound (pre-m01), and thereby a compound (m02) was obtained.

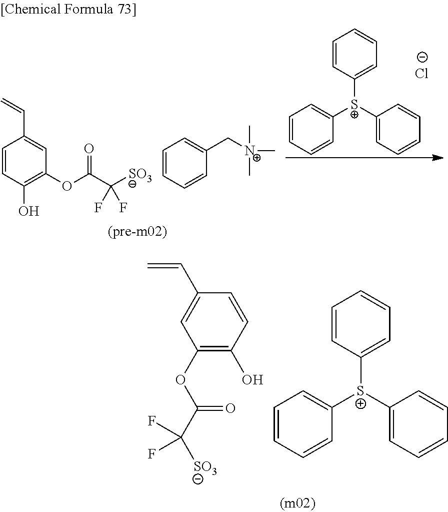

The obtained compound (m02) was subjected to NMR measurement, and the structure thereof was identified from the following analysis results.

Compound (m02)

¹H-NMR (DMSO-d6, 400 MHz): δ (ppm)=5.25 m, 5.76 m, 6.72 d, 6.89 s, 6.99 d, 7.33 m, 7.40 m, 9.48 s

### Synthesis Example 3A: Synthesis of Compound (m03)

A compound (m03) was obtained in the same manner as the synthesis of the compound (m01) except that the raw material was changed.

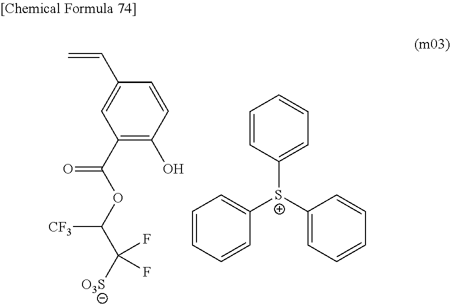

The obtained compound (m03) was subjected to NMR measurement, and the structure thereof was identified from the following analysis results.

Compound (m03)

¹H-NMR (DMSO-d6, 400 MHz): δ (ppm)=5.26 dd, 5.76 dd, 6.72 m, 6.87 d, 7.19 s, 7.33 m, 7.65 m, 8.20 m, 15.25 s

### Synthesis Example 4A: Synthesis of Compound (m04)

A compound (m04) was obtained in the same manner as the synthesis of the compound (m02) except that the raw material was changed.

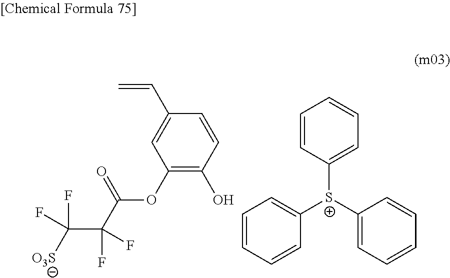

The obtained compound (m04) was subjected to NMR measurement, and the structure thereof was identified from the following analysis results.

Compound (m04)

¹H-NMR (DMSO-d6, 400 MHz): δ (ppm)=5.26 dd, 5.76 dd, 6.72 m, 6.87 d, 7.17 s, 7.33 m, 7.66 m, 15.25 s

### Synthesis Example 5A: Synthesis of Compound (m05)

### (Synthesis of Compound (Pre-m05-1))

50.0 g of 2,5-dihydroxybenzoic acid, 250.0 g of methylene chloride, and 34.5 g of triethylamine were charged into a 500 mL four-necked flask at room temperature, and the mixture was stirred and dissolved. Next, the resultant solution was cooled to 10° C. or less by ice cooling. 32.2 g of methacrylic acid chloride was dissolved in 100.0 g of methylene chloride and added dropwise. The temperature was raised to room temperature, and aging was performed for 3 hours. 240.0 g of ion-exchanged water was charged into the four-necked flask and stirred. After stopping the stirring, the mixture was transferred to a separatory funnel, and the aqueous layer was discharged. Furthermore, washing with 250.0 g of ion exchange was performed. The organic layer was concentrated with a rotary evaporator to obtain 56.7 g of an oily substance. Subsequently, the oily substance was dissolved in 173.0 g of methanol and cooled to obtain 36.0 g of a compound (pre-m05-1) of a white crystal.

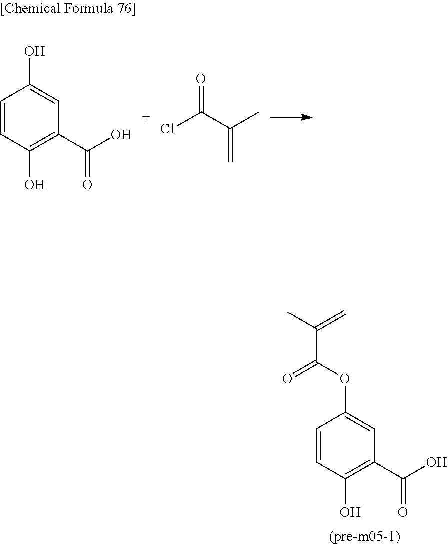

### (Synthesis of Compound (Pre-m05-2))

30.0 g of the compound (pre-m05-1) and 120.0 g of tetrahydrofuran were charged into a 1,000 mL four-necked flask at room temperature, and the mixture was stirred and dissolved. 30.6 g of 1,1′-carbonyldiimidazole was charged into the four-necked flask and stirred. 54.6 g of sulfonate and 25.7 g of diazabicycloundecene were dissolved in 120 g of tetrahydrofuran, and the solution was charged into the four-necked flask and stirred. The temperature was raised to 60° C. in a water bath, and aging was performed for 4 hours. The mixture was cooled to 30° C. in an ice bath. 240.0 g of ion-exchanged water and 240.0 g of methylene chloride were charged into the four-necked flask and stirred. After stopping the stirring, the mixture was transferred to a separatory funnel, and the aqueous layer was discharged. Furthermore, washing with 240.0 g of ion exchange was performed. The organic layer was concentrated with a rotary evaporator to obtain 52.6 g of a compound (pre-m05-2) of an oily substance.

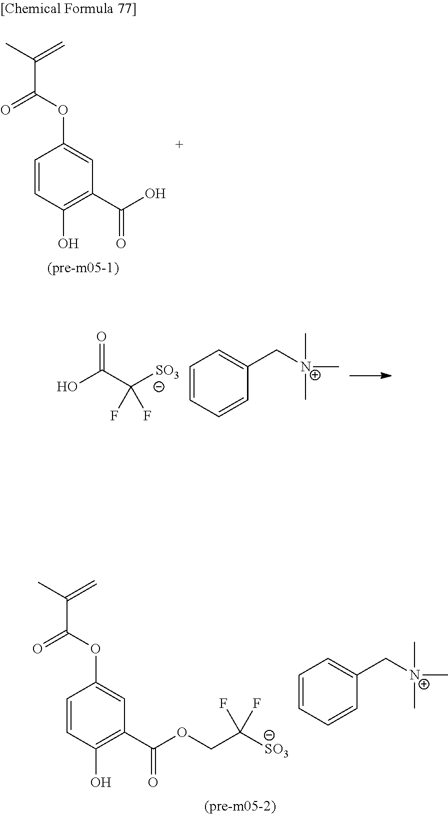

(Synthesis of Compound (m05))

A salt exchange reaction was performed in the same manner as in the synthesis of the compound (m01), except that the compound (pre-m05-2) was used instead of the compound (pre-m01), and thereby a compound (m05) was obtained.

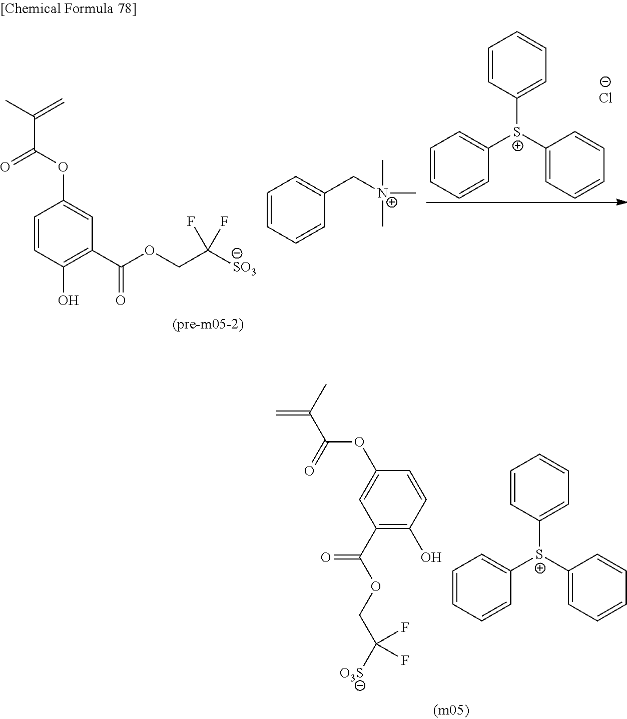

The obtained compound (m05) was subjected to NMR measurement, and the structure thereof was identified from the following analysis results.

Compound (m05)

¹H-NMR (DMSO-d6, 400 MHz): δ (ppm)=5.25 m, 5.76 m, 6.72 m, 6.86 d, 6.99 d, 7.33 m, 7.40 m, 9.48 s

### Synthesis Examples 6A to 11A: Synthesis of Compounds (m06) to (m013)

The following compounds (m06) to (m013) were obtained in the same manner as the synthesis of the compound (m01) except that the raw material was changed.

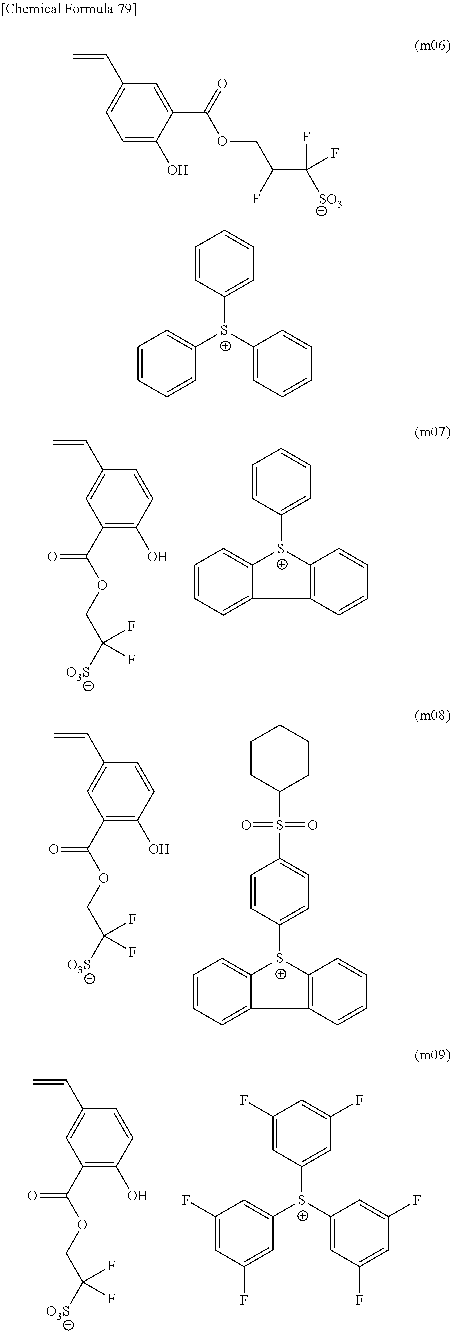

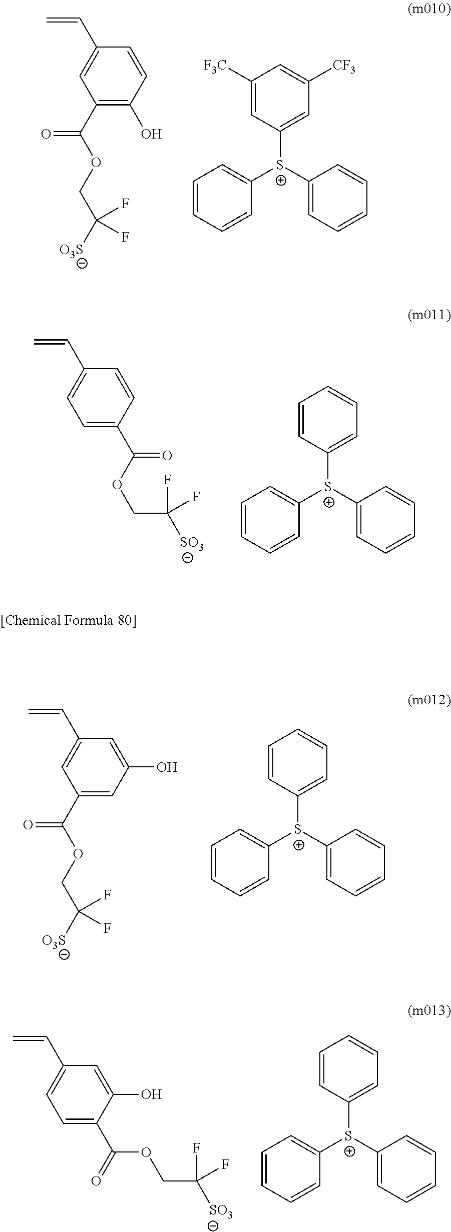

The obtained compound (m012) and obtained compound (m013) were subjected to NMR measurement, and the structures thereof were identified from the following analysis results.

Compound (m012)

¹H-NMR (DMSO-d6, 400 MHz): δ (ppm)=5.13 (2H, dd), 5.25 (1H, t), 5.76 (1H, dd), 6.72 (1H, m), 6.91 (2H, m), 7.24 (1H, s), 7.35 (15H, m), 9.45 (1H, s)

¹H-NMR (DMSO-d6, 400 MHz): δ (ppm)=5.13 (2H, dd), 5.25 (1H, t), 5.76 (1H, dd), 6.72 (1H, m), 6.80 (1H, s), 7.01 (1H, d), 7.35 (15H, m), 7.79 (1H, d), 16.47 (1H, s)

### Synthesis Example 4B: Synthesis of Polymer X04

26.9 g of the compound (m11), 22.8 g of the compound (m01), and 2.4 g of V-601 were dissolved in 100.0 g of MEK, and the mixture was stirred at 80° C. for 5 hours under a nitrogen atmosphere. 1,200 g of heptane was added to the reaction solution to produce a precipitate, and the precipitate was filtered to obtain crystals. The crystals were dried in a vacuum overnight to obtain a polymer X04. The weight-average molecular weight of the obtained polymer X04 in terms of polystyrene, determined by GPC measurement, was 6,700, and the polydispersity was 1.73.

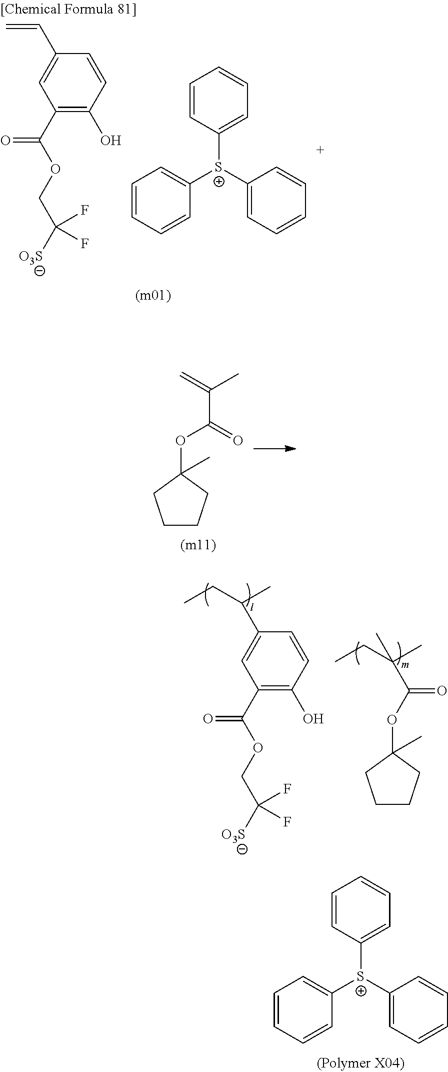

### Synthesis Example 12B: Synthesis of Polymer X12

18.3 g of the compound (m01), 19.5 g of the compound (m11), 3.8 g of (m101), and 2.4 g of V-601 were dissolved in 100.0 g of MEK, and the mixture was stirred at 80° C. for 5 hours under a nitrogen atmosphere.

1,200 g of heptane was added to the reaction solution to produce a precipitate, and the precipitate was filtered to obtain crystals.

The crystals were dried in a vacuum overnight to obtain a target product.

The weight-average molecular weight of the obtained polymer in terms of polystyrene, determined by GPC measurement, was 7,100, and the polydispersity was 1.74.

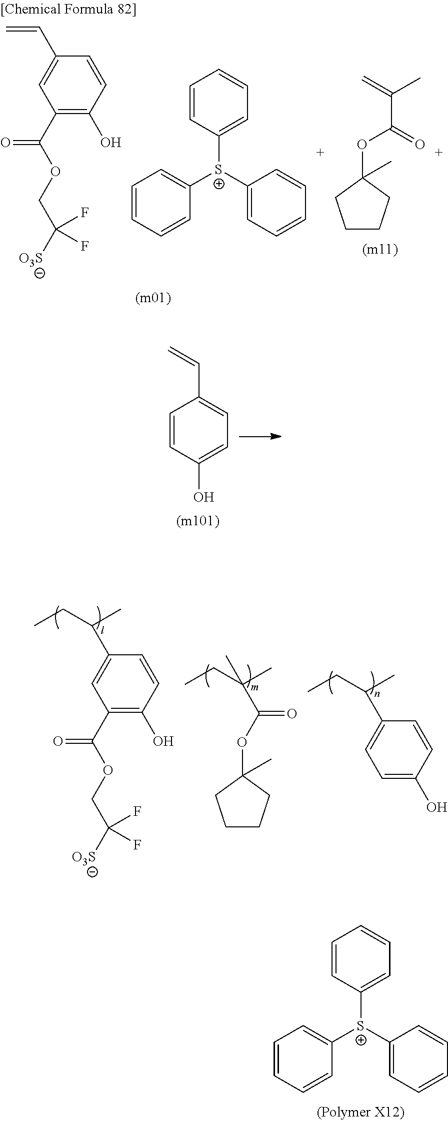

### Synthesis Examples 1B to 3B, 5B to 11B, and 13B to 22B: Synthesis of Polymers X01 to X03, X05 to X11, and X13 to X22

The compounds shown in Table 1 were used at the molar ratios shown in Table 1 to synthesize the polymers X01 to X03, X05 to X11, X20, X21, and X22 in the same manner as in Synthesis Example 4B.

In addition, the compounds shown in Table 1 were used at the molar ratios shown in Table 1 to synthesize the polymers X13 to X19 in the same manner as in Synthesis Example 12B.

For the obtained polymers X01 to X22, the mass-average molecular weight (Mw) in terms of the standard polystyrene value and the polydispersity (PDI) determined by GPC measurement are shown in Table 1.

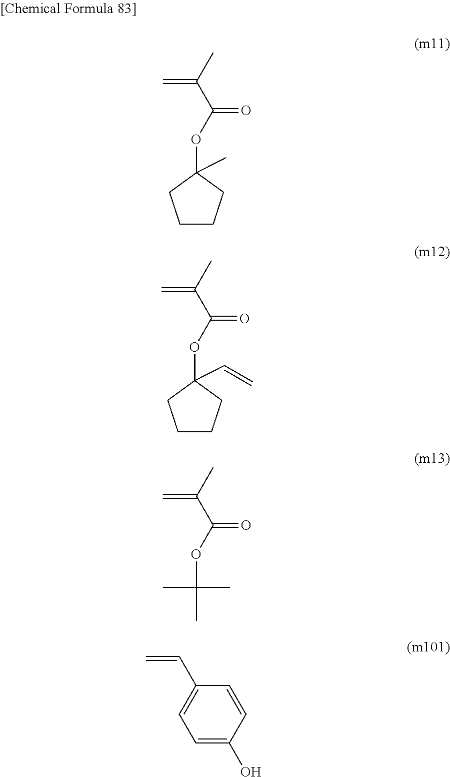

TABLE 1

| Polymer | | | Constitutional unit (a0) | | Constitutional unit (a1) | | Constitutional unit (a10) | |
|---|---|---|---|---|---|---|---|---|
| Polymer | | | | Introduction | | Introduction | | Introduction |
| name | Mw | PDI | Monomer | ratio (wt %) | Monomer | ratio (wt %) | Monomer | ratio (wt %) |
| X01 | 6,900 | 1.74 | (m01) | 15.1 | (a11) | 84.9 | — | — |
| X02 | 7,300 | 1.75 | (m01) | 27.4 | (a11) | 72.6 | — | — |
| X03 | 6,800 | 1.72 | (m01) | 37.4 | (a11) | 62.6 | — | — |
| X04 | 6,700 | 1.73 | (m01) | 45.9 | (a11) | 54.1 | — | — |
| X05 | 7,200 | 1.75 | (m01) | 53.1 | (a11) | 46.9 | — | — |
| X06 | 7,000 | 1.71 | (m01) | 59.2 | (a11) | 40.8 | — | — |
| X07 | 6,900 | 1.73 | (m02) | 45.3 | (a11) | 54.7 | — | — |
| X08 | 7,200 | 1.75 | (m03) | 47.1 | (a11) | 52.9 | — | — |
| X09 | 6,900 | 1.72 | (m04) | 47.4 | (a11) | 52.6 | — | — |
| X10 | 7,000 | 1.73 | (m05) | 47.7 | (a11) | 52.3 | — | — |
| X11 | 7,300 | 1.69 | (m06) | 47.2 | (a11) | 52.8 | — | — |
| X12 | 6,900 | 1.74 | (m01) | 40.6 | (a11) | 50.9 | (a101) | 8.5 |
| X13 | 7,400 | 1.71 | (m07) | 40.5 | (a11) | 50.9 | (a101) | 8.6 |
| X14 | 7,000 | 1.72 | (m08) | 45.7 | (a11) | 46.5 | (a101) | 7.8 |
| X15 | 6,700 | 1.74 | (m09) | 44.3 | (a11) | 47.7 | (a101) | 8 |
| X16 | 7,200 | 1.7 | (m010) | 45.8 | (a11) | 46.4 | (a101) | 7.8 |
| X17 | 7,100 | 1.72 | (m01) | 39.2 | (a12) | 52.6 | (a101) | 8.2 |
| X18 | 7,100 | 1.72 | (m01) | 44.1 | (a13) | 46.7 | (a101) | 9.3 |
| X19 | 6,800 | 1.67 | (m011) | 34.4 | (a11) | 49.9 | (a101) | 15.7 |
| X20 | 6,800 | 1.69 | — | — | (a11) | 76.6 | (a101) | 23.4 |
| X21 | 6,900 | 1.75 | (m012) | | (a11) | | — | — |
| X22 | 7,000 | 1.74 | (m013) | | (a11) | | — | — |

The structures of the polymers X01 to X22 are shown below. In addition, the copolymerization compositional ratio (the proportion (molar ratio) of each constitutional unit in the polymer compound) of the polymer compound, which is obtained by ¹³C-NMR is also shown.
Polymer X01: l/m=5/95
Polymer X02: l/m=10/90
Polymer X03: l/m=15/85
Polymer X04: l/m=20/80
Polymer X05: l/m=25/75
Polymer X06: l/m=30/70

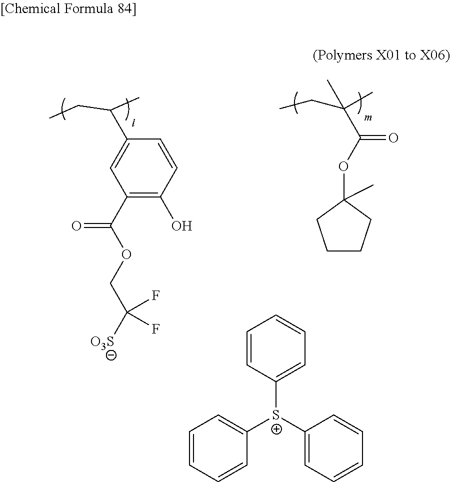

Polymer X07: l/m=20/80
Polymer X08: l/m=20/80
Polymer X09: l/m=20/80
Polymer X10: l/m=20/80
Polymer X11: l/m=20/80

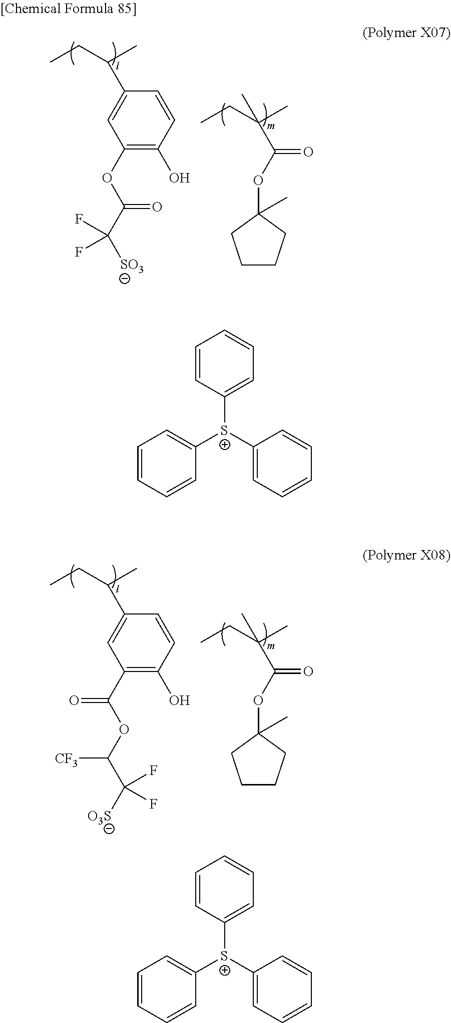

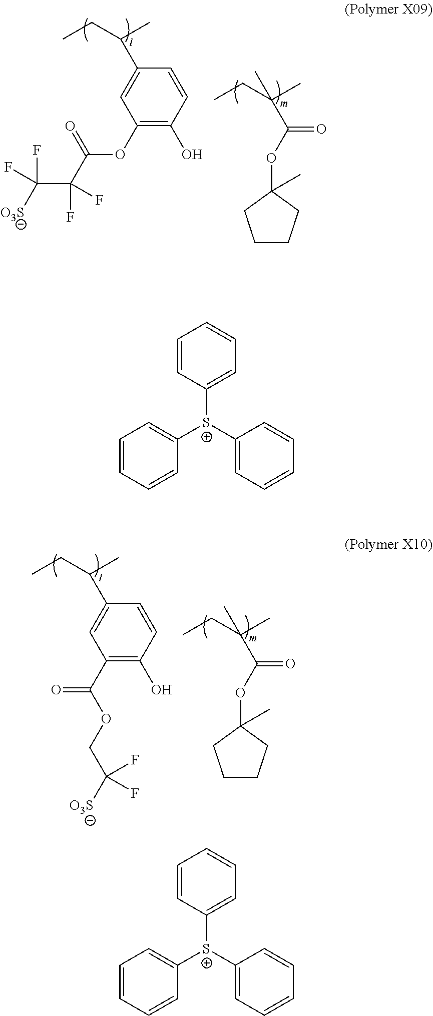

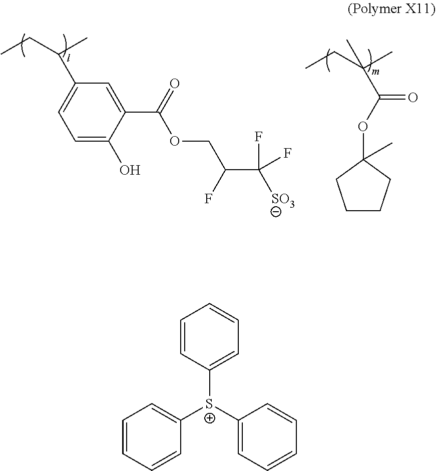

Polymer X12: l/m/n=16/68/16
Polymer X13: l/m/n=16/68/16
Polymer X14: l/m/n=16/68/16

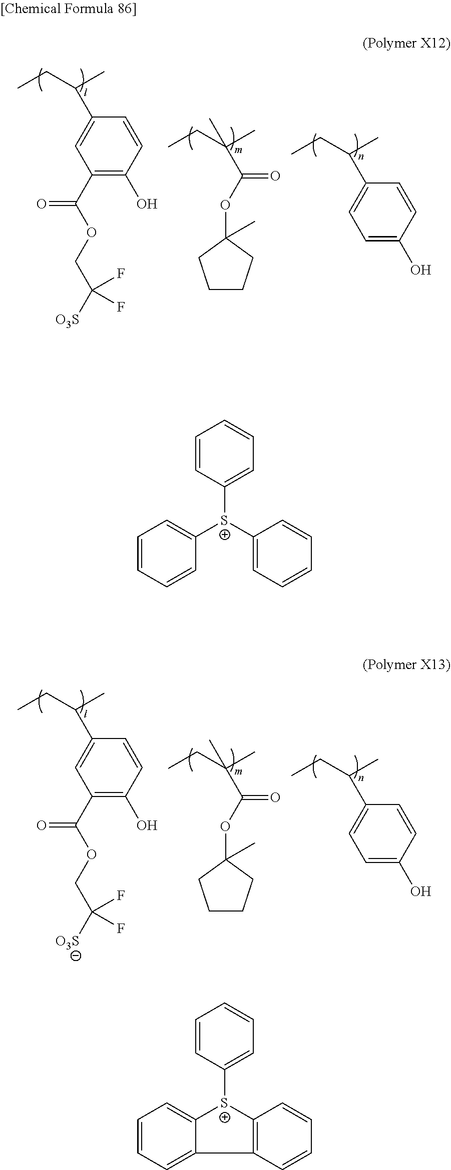

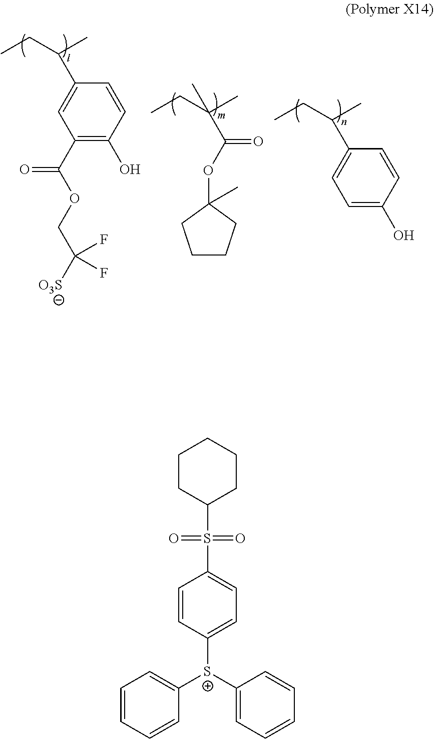

Polymer X15: l/m/n=16/68/16

Polymer X16: l/m/n=16/68/16

Polymer X17: l/m/n=16/68/16

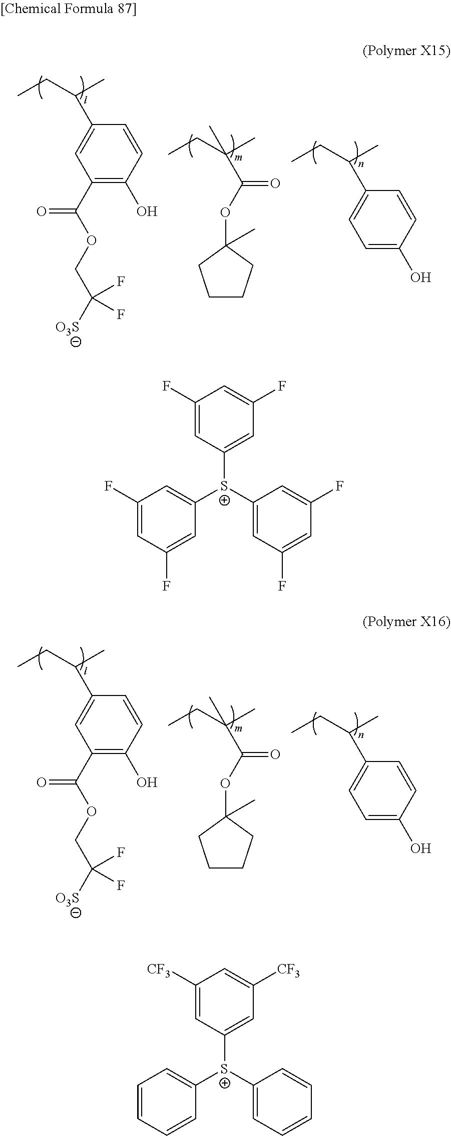

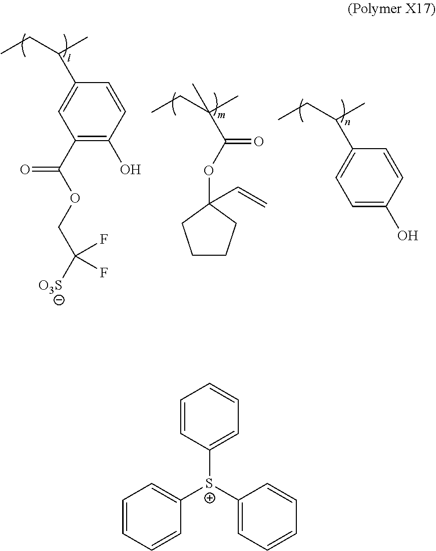

Polymer X18: l/m/n=16/68/16
Polymer X19: l/m/n=16/68/16
Polymer X20: l/m=30/70

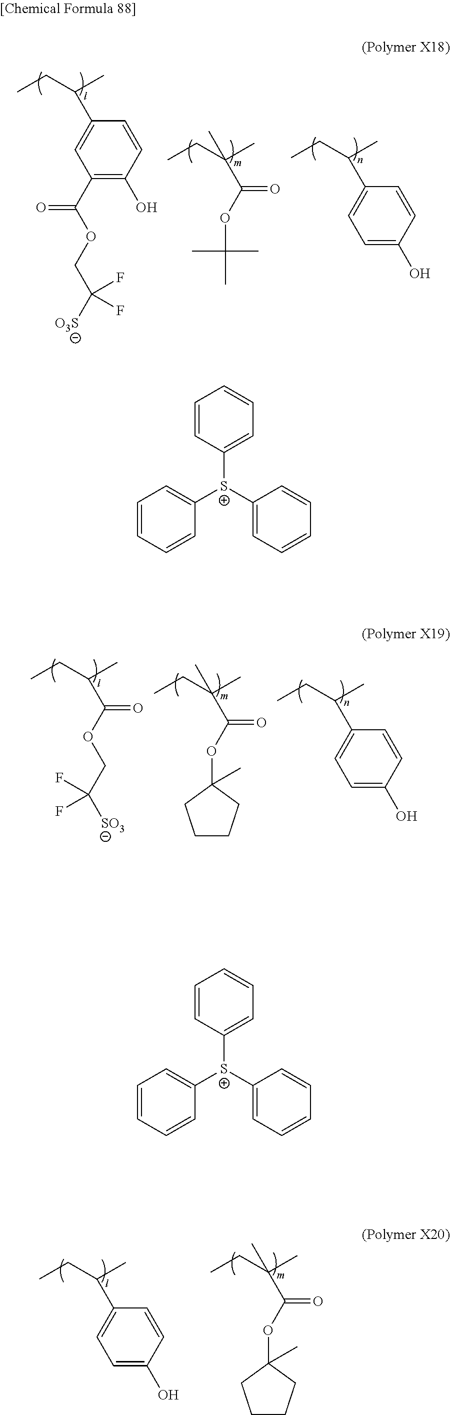

Polymer X21: l/m=20/80
Polymer X22: l/m=20/80

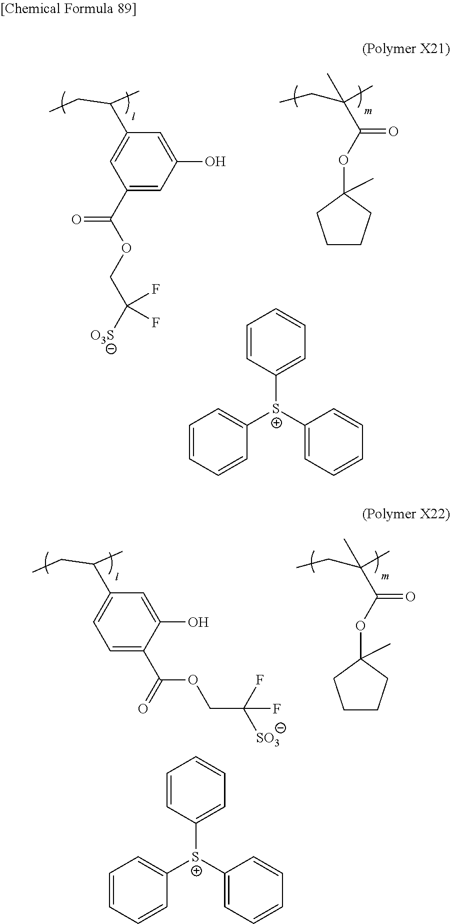

### Preparation of Resist Composition

### Examples 1 to 21 and Comparative Examples 1 and 2

Each of the components shown in Tables 2 and 3 was mixed and dissolved to prepare a resist composition of each Example.

TABLE 2

| | Component | Component | Component | Eop | LWR |
|---|---|---|---|---|---|
| | (A) | (D) | (S) | (μC/cm²) | (nm) |
| Example | (A)-1 | (D)-1 | (S)-1 | 109 | 3.1 |
| 1 | [100] | [5] | [8000] | | |
| Example | (A)-2 | (D)-1 | (S)-1 | 90 | 3.1 |
| 2 | [100] | [5] | [8000] | | |
| Example | (A)-3 | (D)-1 | (S)-1 | 84 | 3.2 |
| 3 | [100] | [5] | [8000] | | |
| Example | (A)-4 | (D)-1 | (S)-1 | 82 | 3.1 |
| 4 | [100] | [5] | [8000] | | |
| Example | (A)-5 | (D)-1 | (S)-1 | 82 | 3.7 |
| 5 | [100] | [5] | [8000] | | |
| Example | (A)-6 | (D)-1 | (S)-1 | 81 | 3.9 |
| 6 | [100] | [5] | [8000] | | |
| Example | (A)-7 | (D)-1 | (S)-1 | 85 | 3.2 |
| 7 | [100] | [5] | [8000] | | |
| Example | (A)-8 | (D)-1 | (S)-1 | 85 | 3.1 |
| 8 | [100] | [5] | [8000] | | |
| Example | (A)-9 | (D)-1 | (S)-1 | 83 | 3.2 |
| 9 | [100] | [5] | [8000] | | |
| Example | (A)-10 | (D)-1 | (S)-1 | 84 | 3.1 |
| 10 | [100] | [5] | [8000] | | |
| Example | (A)-11 | (D)-1 | (S)-1 | 82 | 3.2 |
| 11 | [100] | [5] | [8000] | | |
| Example | (A)-12 | (D)-1 | (S)-1 | 82 | 3.2 |
| 12 | [100] | [5] | [8000] | | |
| Example | (A)-13 | (D)-1 | (S)-1 | 82 | 3.3 |
| 13 | [100] | [5] | [8000] | | |
| Example | (A)-14 | (D)-1 | (S)-1 | 84 | 3.3 |
| 14 | [100] | [5] | [8000] | | |
| Example | (A)-15 | (D)-1 | (S)-1 | 77 | 3 |
| 15 | [100] | [5] | [8000] | | |
| Example | (A)-16 | (D)-1 | (S)-1 | 77 | 2.9 |
| 16 | [100] | [5] | [8000] | | |
| Example | (A)-17 | (D)-1 | (S)-1 | 79 | 3.2 |
| 17 | [100] | [5] | [8000] | | |
| Example | (A)-18 | (D)-1 | (S)-1 | 83 | 3.4 |
| 18 | [100] | [5] | [8000] | | |

TABLE 3

| | Component | Component | Component | Component | Eop | LWR |
|---|---|---|---|---|---|---|
| | (A) | (B) | (D) | (S) | (μC/cm²) | (nm) |
| Example 19 | (A)-2 | (B)-1 | (D)-1 | (S)-1 | 82 | 4.1 |
| | [100] | [15] | [5] | [8000] | | |
| Example 20 | (A)-21 | — | (D)-1 | (S)-1 | 82 | 3.1 |
| | [100] | | [5] | [8000] | | |
| Example 21 | (A)-22 | — | (D)-1 | (S)-1 | 83 | 3.2 |
| | [100] | | [5] | [8000] | | |
| Comparative | (A)-19 | — | (D)-1 | (S)-] | 94 | 4.5 |
| Example 1 | [100] | | [5] | [8000] | | |
| Comparative | (A)-20 | (B)-1 | (D)-1 | (S)-1 | 87 | 5.4 |
| Example 2 | [100] | [30] | [5] | [8000] | | |

In Tables 2 and 3, each abbreviation has the following meaning. A numerical value in the brackets is a blending amount (part by mass).
(A)-1 to (A)-22: the above-described polymers X01 to X22
(B)-1: an acid generator represented by Chemical Formula (B-1).
(D)-1: an acid diffusion control agent represented by Chemical Formula (D-1).
(S)-1: A mixed solvent of propylene glycol monomethyl ether acetate/propylene glycol monomethyl ether=60/40 (mass ratio)

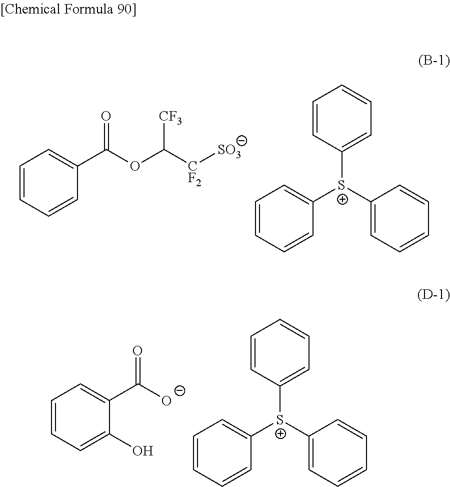

### <Formation of Resist Pattern>

The resist composition of each Example was applied onto an 8-inch silicon substrate which had been subjected to a hexamethyldisilazane (HMDS) treatment using a spinner, the coated wafer was subjected to a pre-baking (PAB) treatment on a hot plate at a temperature of 110° C. for 60 seconds so that the coated wafer was dried to form a resist film having a film thickness of 50 nm. Next, drawing (exposure) was carried out on the resist film by using an electron beam lithography apparatus JEOL-JBX-9300FS (manufactured by JEOL Ltd.), with the target size set to a 1:1 line and space pattern (hereinafter, referred to as “LS pattern”) of a line width of 50 nm, at an acceleration voltage of 100 kV. Thereafter, a post-exposure baking (PEB) treatment was performed thereon at 100° C. for 60 seconds. Subsequently, alkali development was performed at 23° C. for 60 seconds using a 2.38% by mass tetramethylammonium hydroxide (TMAH) aqueous solution “NMD-3” (trade name, manufactured by TOKYO OHKA KOGYO CO., LTD.). Thereafter, water rinsing was performed for 15 seconds using pure water. As a result, a 1:1 LS pattern having a line width of 50 nm was formed.

### <Evaluation of Optimum Exposure Amount (Eop)>

The optimum exposure amount Eop (μC/cm²) at which an LS pattern having a target size was formed by the above-described method for forming a resist pattern was determined. The results are shown in Tables 2 and 3.

### <Evaluation of LWR>

3σ of the LS pattern formed in <Formation of resist pattern> described above, which is a scale indicating LWR, was determined. This is shown as “LWR (nm)”. “3σ” was determined as a triple value (3σ) (unit: nm) of the standard deviation (σ) obtained by measuring 400 line positions in the longitudinal direction of the line with a scanning electron microscope (acceleration voltage: 800V, trade name: S-9380, manufactured by Hitachi High-Tech Corporation). The results are shown in Tables 2 and 3.

The smaller the value of 3σ is, the smaller the roughness in the line side wall is, which means an LS pattern having a more uniform width was obtained.

As listed in Tables 2 and 3, it was confirmed that the resist compositions of Examples 1 to 21 were capable of achieving high sensitivity and had excellent LWR in the resist pattern formation.

Although preferable examples of the present invention have been described above, the present invention is not limited to these examples. Configurations can be added, omitted, and replaced, and other modifications can be made within a range not departing from the gist of the present invention. The present invention is not limited by the above description, but only by the scope of the appended claims.

What is claimed is:

1. A resist composition which generates an acid upon exposure to light and whose solubility in a developing solution is changed under action of an acid, the resist composition comprising:
a resin component (A1) whose solubility in a developing solution is changed under action of an acid,
wherein the resin component (A1) has a constitutional unit (a0) derived from a compound represented by General Formula (a0), 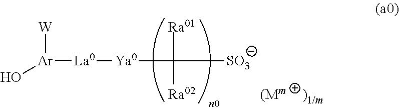
wherein W represents a polymerizable group, Ar represents an aromatic hydrocarbon group which may have a substituent, —OH represents a hydroxy group, La⁰ represents a divalent linking group, Ya⁰ represents a single bond or a divalent linking group which may have a hetero atom, Ra⁰¹ and Ra² each independently represents a hydrogen atom, a fluorine atom, or a fluorinated alkyl group, n0 represents an integer in a range of 1 to 4, m represents an integer of 1 or more, and M^{m+} represents an m-valent organic cation.

2. The resist composition according to claim 1, wherein the resin component (A1) further has comprises a constitutional unit (a1) that contains an acid decomposable group whose polarity is increased under action of an acid.

3. The resist composition according to claim 1,
wherein the resin component (A1) further comprises a constitutional unit (a10) represented by General Formula (a10-1), 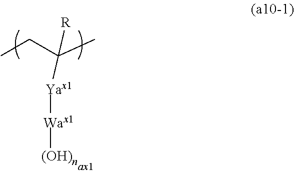
wherein R represents a hydrogen atom, an alkyl group having 1 to 5 carbon atoms, or a halogenated alkyl group having 1 to 5 carbon atoms, Ya^{x1} represents a single bond or a divalent linking group, Wa^{x1} represents an aromatic hydrocarbon group which may have a substituent, and nₐₓ₁ represents an integer of 1 or more.

4. A method for forming a resist pattern, comprising:
forming a resist film on a support using the resist composition according to claim 1;
exposing the resist film to light; and
developing the resist film exposed to light to form a resist pattern.

5. The method for forming a resist pattern according to claim 4,
wherein the resist film is exposed to an extreme ultraviolet ray or an electron beam).

6. A polymer compound comprising a constitutional unit (a0) derived from a compound represented by General Formula (a0), 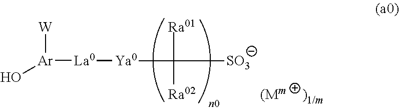
wherein W is a polymerizable group, Ar is an aromatic hydrocarbon group which may have a substituent, —OH is a hydroxy group, La⁰ represents a divalent linking group, Ya⁰ represents a single bond or a divalent linking group which may have a hetero atom, Ra⁰¹ and Ra⁰² each independently represents a hydrogen atom, a fluorine atom, or a fluorinated alkyl group, n0 represents an integer in a range of 1 to 4, m represents an integer of 1 or more, and M^{m+} represents an m-valent organic cation.

7. The polymer compound according to claim 6, further comprising a constitutional unit (a1) that contains an acid decomposable group whose polarity is increased under action of an acid.

8. The polymer compound according to claim 6, further comprising a constitutional unit (a10) represented by General Formula (a10-1), 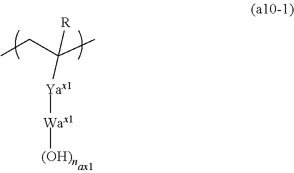
wherein R represents a hydrogen atom, an alkyl group having 1 to 5 carbon atoms, or a halogenated alkyl group having 1 to 5 carbon atoms, Ya^{x1} represents a single bond or a divalent linking group, Wa^{x1} represents an aromatic hydrocarbon group which may have a substituent, and nₐₓ₁ represents an integer of 1 or more.

9. A compound represented by General Formula (a0), 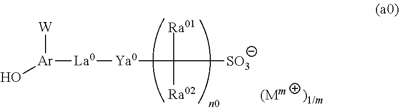
wherein W is a polymerizable group, Ar is an aromatic hydrocarbon group which may have a substituent, —OH is a hydroxy group, La⁰ represents a divalent linking group, Ya⁰ represents a single bond or a divalent linking group which may have a hetero atom, Ra⁰¹ and Ra² each independently represent a hydrogen atom, a fluorine atom, or a fluorinated alkyl group, n0 represents an integer in a range of 1 to 4, m represents an integer of 1 or more, and M^{m+} represents an m-valent organic cation].
